(19) United States
(12) Patent Application Publication
(10) Pub. No.: US 2022/0033811 A1
(43) Pub. Date: Feb. 3, 2022

(54) METHOD AND KIT FOR PREPARING COMPLEMENTARY DNA

(71) Applicants: Michael HAGEMANN-JENSEN, Stockholm (SE); Omid FARIDANI, Botany, New South Wales (AU); BIOBLOXX AB, Huddinge (SE)

(72) Inventors: Michael HAGEMANN-JENSEN, Stockholm (SE); Omid FARIDANI, Botany, New South Wales (AU); Rickard SANDBERG, Huddinge (SE)

(21) Appl. No.: 17/276,718

(22) PCT Filed: Dec. 27, 2019
(86) PCT No.: PCT/IB2019/001386

§ 371 Date: Mar. 16, 2021

(30) Foreign Application Priority Data
Dec. 28, 2018 (SE) 1851672-4

(51) Int. Cl. C12N 15/10; C12Q 1/6806

(52) U.S. Cl. CPC C12N 15/1096; C12Q 1/6806

(57) ABSTRACT

cDNA is prepared by hybridizing a cDNA synthesis primer to an RNA molecule and synthesizing a cDNA strand complementary to at least a portion of the RNA molecule to form an RNA-cDNA intermediate. A template switching reaction is performed by contacting the RNA-cDNA intermediate with a template switching oligonucleotide (TSO) under conditions suitable for extension of the cDNA strand using the TSO as template to form an extended cDNA strand complementary to the at least a portion of the RNA molecule and the TSO. The TSO comprises an amplification primer site, an identification tag, a UMI and multiple predefined nucleotides.

### CROSS-REFERENCE TO RELATED APPLICATIONS

Pursuant to 35 U.S.C. § 119(e), this application claims priority to the filing date of the Swedish Provisional Patent Application Serial No. 1851672-4 filed Dec. 28, 2018; the disclosure of which application is herein incorporated by reference.

### TECHNICAL FIELD

The present invention generally relates to complementary deoxyribonucleic acid (cDNA) synthesis, and in particular to method and kit for preparing cDNA suitable for sequencing.

### BACKGROUND

Single cell ribonucleic acid sequencing (scRNA-seq) has dramatically improved the ability to molecularly profile large numbers of cells in order to identify and enumerate, for instance, cell types, sub-types, cell states and heterogeneous responses to different signals. Essentially all scRNA-seq methods profile RNA molecules comprising a poly-A tail, e.g., messenger RNA (mRNA) molecules, and can generally be divided into two main methods.

The first main method profiles a small stretch of bases at either the 5′ end or the 3′ end of the mRNA molecules with high cellular throughput. These methods include single-cell tagged reverse transcription sequencing (STRT-seq) [1], single cell sequencing (CEL-seq) [2], massively parallel single-cell RNA sequencing (MARS-seq) [3], 10X Genomics single cell RNA sequencing [4], split-pool ligation-based transcriptome sequencing (SPLiT-seq) [5] and single-cell combinatorial indexing RNA sequencing (sci-RNA-seq) [6]. All of these methods utilize a unique molecular identifier (UMI) that is present in the oligo-dT primer or a template switching oligonucleotide (TSO). The UMI is used to remove the biased amplification effect of polymerase chain reaction (PCR). These methods thereby enable counting the mRNA molecules present before amplification.

The second main method fragments cDNA molecules for a subsequent capture of cDNA fragments derived from the complete mRNA molecules, thus providing up to full-length transcript coverage. Notably methods include Smart-seq [7] and Smart-seq2 [8, 10, 11], which provide the most sensitive information of single-cell transcriptomes, i.e., captures the largest fraction of RNAs present in the cells. However, these methods are not compatible with UMIs and cannot therefore count mRNA molecules in single cells.

There is still need for improvements within the field of RNA sequencing and in particular scRNA-seq.

### SUMMARY

It is a general objective to prepare cDNA that is suitable for sequencing.

This and other objectives are met by embodiments as defined herein.

The present invention relates to a method and a kit for preparing cDNA as defined in the independent claims. Further embodiments of the invention are defined in the dependent claims.

The method for preparing cDNA comprises hybridizing a cDNA synthesis primer to an RNA molecule and synthesizing a cDNA strand complementary to at least a portion of the RNA molecule to form an RNA-cDNA intermediate. The method also comprises performing a template switching reaction by contacting the RNA-cDNA intermediate with a TSO under conditions suitable for extension of the cDNA strand using the TSO as template to form an extended cDNA strand complementary to the at least a portion of the RNA molecule and the TSO. According to the invention, the TSO comprises an amplification primer site, an identification tag, a UMI and multiple predefined nucleotides.

The kit for preparing cDNA comprises a cDNA synthesis primer configured to hybridize to an RNA molecule to enable synthesis of a cDNA strand complementary to at least a portion of the RNA molecule to form an RNA-cDNA intermediate. The kit also comprises a TSO comprising an amplification primer site, an identification tag, a UMI and multiple predefined nucleotides. The TSO is configured to act as a template in a template switching reaction comprising extension of the DNA strand to form an extended cDNA strand complementary to the at least a portion of the RNA molecule and the TSO.

The present invention enables usage of UMIs and therefore removes amplification bias and still provides up to full-length transcript coverage. This is possible by the usage of the TSO of the invention that introduces an UMI into the extended cDNA strands.

### BRIEF DESCRIPTION OF THE DRAWINGS

The embodiments, together with further objects and advantages thereof, may best be understood by making reference to the following description taken together with the accompanying drawings, in which:

FIGS. 1A and 1B illustrate single cell RNA sequencing library construction for combined full-length transcript coverage and UMIs. Individual cells were lysed in individual reaction vessels (e.g., individual tubes, wells of a multi-well plate, nanowells or microwells or chambers of a microfluidic device or droplets) and subject to reverse transcription and template switching. Resulting first strand cDNAs were pre-amplified, during which full Nextera P5 adapter sequence was inserted at the 5′ end. Double-stranded cDNA was subject to tagmentation, PCR-mediated indexing and ILLUMINA® sequencing.

FIG. 2 illustrates boxplots showing improved gene detection with the invention.

FIG. 3, panels A and B illustrate detailed RNA biotype detection with the invention and prior art Smart-seq2.

FIG. 4 illustrates control of the levels of 5′ end reads and internal reads.

FIG. 5, panels A to C illustrate cDNA length distributions of differential tagmented cDNA.

FIG. 6, panels A to C illustrate increased gene detection by altering reaction conditions and experimental additives.

FIG. 7, panels A and B illustrate the read coverage across RNA molecules for internal reads and UMI-containing 5′-end reads, respectively.

FIG. 8 is a flow chart illustrating a method for preparing cDNA according to an embodiment.

FIG. 9. (a) Library strategy for an embodiment of the invention, referred to as Smart-seq3. PolyA+ RNA molecules are reverse transcribed and template switching is carried out at the 5′ end. After PCR preamplification, tagmentation via Tn5 introduces near-random cuts in the cDNA, producing 5′ UMI-tagged fragments and internal fragments spanning the whole gene body. (b) Gene body coverage averaged over HEK293FT (n=96) cells sequenced with the Smart-seq3 protocol. Shown is the mean coverage of UMI reads (green) and internal reads (blue) shaded by the standard deviation. (c) Effect of tagmentation conditions on the fraction of UMI-containing reads (16 HEK293FT cells per condition). Left panel: varying Tn5 with constant 200 pg cDNA input. Right panel: varying cDNA input with constant 0.5ul Tn5. (d) Gene detection sensitivity for Smart-seq2 (44 cells) and Smart-seq3 (88 cells), downsampled to 1 million raw reads per HEK293FT cell. Shown are number of genes detected over 0 or 1 RPKM. P-value was computed as a two-sided t-test. (e) Reproducibility in gene expression quantification across HEKF293FT cells for Smart-seq2 (44 cells) and Smart-seq3 (88 cells) at RPKM and UMI level. Shown are adjusted r\hat{ }2 for all pairwise cell to cell linear model fits in libraries downsampled to 1 million reads per cell. (f) Sensitivity to detect RNA molecules in Smart-seq3 shown by summarizing the number of unique error-corrected UMI sequences and genes detected per HEK293FT cell. Colors indicate the per cell downsampling depth ranging from 10.000 (n=24 cells) to 750.000 (n=16 cells) UMI-containing sequencing reads. (g) Violin plots summarizing the number of molecules detected per cell with Smart-seq2-UMI, Smart-seq3 and using smRNA-FISH for four X chromosomal genes (Hdac6, Igbp1, Mpp1 and Msl3). (h) Estimating the percent of smRNA-FISH molecules that were detected in cells using Smart-seq2-UMI and Smart-seq3. Shown are means and 95% confidence intervals.

FIG. 10. Overview of sequenced conditions and iterations of Smart-seq3. Each row shows a tested reaction condition and the number of genes detected in individual HEK293FT cells at 1M raw fastq reads. The numbers of individual cells that contained at least one million sequenced reads per condition are listed on the right. Several earlier versions of Smart-seq2 with elements of Smart-seq3 chemistry are included as “Smart-seq2.5” in this figure. The exact reaction conditions per row are listed in Table 4.

FIG. 11. Effects of salts, PEG and additives on Smart-seq3 reverse transcription. (a) Testing the performance of Maxima H-minus reverse transcription reactions on different reaction conditions. For each condition, we summarized boxplots with the number of unique UMIs detected in individual HEK293FT cells at 1M raw fastq reads. We tested reverse transcription in the context of using a NaCl, CsCl or the standard KCl based buffer. Moreover, we evaluated the effects of adding of 5% PEG or 1 mM dCTP (16 cells per condition). (b) Reaction conditions as in (a) summarized against the number of genes identified from 1 million raw UMI-reads per cell (16 cells per condition). (c) Reaction conditions as in (a) summarized against the number of genes identified from 1 million raw reads (sub-sampling from both 5′ UMI and internal reads) per cell (16 cells per condition).

FIG. 12. Improved detection of protein-coding and non-coding RNAs with Smart-seq3. (a) Variants of Smart-seq3 reactions show improved detection of protein coding genes and also genes of different biotypes, including poly-A+ lincRNAs, antisense RNAs, processed pseudogenes, processed transcripts and snoRNAs, compared to Smart-seq2 and earlier experimentations of Smart-seq2 with UMIs (here called “intermediate”). (b) Shows genes detected of similar RNA biotypes by UMI containing reads in Smart-seq2 with UMIs (here called “intermediate”) and Smart-seq3 variants.

FIG. 13. Single-cell RNA counting at allele and Isoform-resolution. (a) Strategy for obtaining allelic and isoform resolved information using Smart-seq3. Red crosses indicate transcript positions with genetic variation between alleles. After tagmentation, UMI fragments are subjected to paired-end sequencing (indicated in green), linking molecule-counting 5′ ends with various gene-body fragments that can cover allele-informative variant positions and spanning isoform-informative splice junctions, thus allowing in silico reconstruction of isoforms and allele of origin. (b) Average percentage of molecules that could be assigned to allele origin based on covered SNPs, from 369 individual CAST/EiJ×C57/BI6J hybrid mouse fibroblasts. Only genes detected in >5% of cells were considered (n=15,158 genes). (c) Effect of transcript length and number of exonic SNPs on allele assignment of RNA molecules. Shown are genes (n=15,158) grouped into 50 2D-bins colored by the average gene-wise percentage of molecules assigned to allele of origin. Inset shows the number of genes per visualized bin. (d) Concordance of allele expression from RNA counting and traditional estimates based on separated expression and allele-fractions from internal reads. Shown are the average CAST allele fractions for 15,158 genes over 369 mouse Fibroblasts. Dots are colored by the local density of data points. (e) Results from linear models that compared direct allelic RNA counting with previous read-based estimates of allelic expression, within each of 369 individual fibroblasts. For each cell (n=369), we computed a linear model fit of CAST allele fraction between direct reconstructed molecule assignment and traditional read-based estimates. Shown are boxplots of the Intercept, slope and rA2 values obtained from each linear model per cell. (f) Demonstrating the improved abilities of Smart-seq3 to infer transcriptional burst kinetics compared to Smart-seq2-UMI (the Smart-seq2 chemistry combined with a UMI in the TSO). Inference was made in F1 CAST/EiJ×C57/BI6J mouse fibroblasts and we show the spearman correlation between the CAST and C57 kinetics across genes for burst size and frequency. Additionally, the x-axis shows the number of genes for which we could reliably infer the bursting kinetics. (g) Summarizing the numbers of RNA molecules (x-axis, log 10) reconstructed to different lengths (in base pairs, y-axis), showing only molecules additionally assigned to a unique transcript isoform. In total, the one million longest reconstructed RNA molecules are shown from one experiment with 369 mouse fibroblasts, with molecules shown in descending order. (h) Sashimi plots visualizing two reconstructed RNA transcripts that supported two distinct transcript isoforms of Cox7a2I (ENSMUST00000167741 in orange, and ENSMUST00000025095 in light blue), observed in a mouse fibroblasts (cell barcode: TTCCGTTCGCGACTAA). (i) Violin plots showing the percentage of detected molecules that could be assigned to a specific Ensembl transcript isoform, per F1 CAST/EiJ×C57/BI6J mouse fibroblast. Reported are the results on all Ensembl genes, or the subset with two or more annotated isoforms (‘multi-isoform genes’). The median percentages of assigned molecules per cell were 52.37% and 41.04% for all and multi-isoform genes, respectively. (j) Visualizing significant strain-specific isoform expression in mouse fibroblasts, colored by chromosomes. Y-axis shows Benjamini-Hochberg corrected p-values (−log 10) from individual Chi-square tests performed per gene evaluating association between allelic origin and isoforms. (k) Visualizing the significant strain-specific isoform expression of Hcfc1r1 in CAST/EiJ and C57/BI6J mouse strains. Violin plots depict isoform expression in mouse fibroblasts, separated per strain and isoform. Top shows the transcript isoform structures.

FIG. 14. Visualization of read-pairs from a single transcribed molecule from Cox7a2 locus in primary fibroblast cell. Visualization of read pairs sequenced from one molecule from the Cox7a2l locus. Top show the exons and introns in the Cox7a2l locus, with genomic coordinates (mm10). Each row show a unique read pair, where oranges boxes show the mapping of sequences onto the genomic loci, dotted lines indicate that the sequences are connected by the read pairs and solid lines represent that the exon-intron junction was captured in the sequenced reads. Note, all read pairs combined span essentially the full transcript, meaning that for this molecule we could reconstruct the full transcript.

FIG. 15. Detailed comparison of burst kinetics inference based on Smart-seq2-UMI and Smart-seq3 data.

(a) Scatter plots showing the burst frequencies inferred for the C57 (x-axis) and CAST (y-axis) alleles for genes in mouse fibroblasts. The left plot show the results based on Smart-seq3 data and the right panel show the results from using Smart-seq2-UMI data. (b) Scatter plots showing the burst sizes inferred for the C57 (x-axis) and CAST (y-axis) alleles for genes in mouse fibroblasts. The left plot show the results based on Smart-seq3 data and the right panel show the results from using Smart-seq2-UMI data.

FIG. 16. Species-mixing and doublets in Smart-seq3.

(a) Scatter plot showing the number of reads that aligned to human (x-axis) and mouse (y-axis) for the complex HCA sample that contained both human, mouse and dog cells. (b) Scatter plot showing the number of reads that aligned to human (x-axis) and dog (y-axis) for the complex HCA sample that contained both human, mouse and dog cells. Few cells show any signal towards more than one genome, demonstrating a very low doublet rate.

FIG. 17 Smart-seq3 analysis of a complex human sample. (a) Dimensionality reduction (U MAP) of 3,890 human cells sequenced with the Smart-seq3 protocol and colored by annotated cell type. (b) Comparison of sensitivity to detect genes between Smart-seq2 and Smart-seq3 in various cell types. Cells were down-sampled to 100k raw reads per cell and t-test p-values are annotated for each pair-wise comparison. (c) Heatmap showing gene expression for selected marker genes that were expressed at statistically significantly different levels in naïve and memory B-cells. Color scale represents normalized and scaled expression values. (d) The percentage of reconstructed RNA molecules that could be assigned to a single Ensembl isoform, separated by cell types. (e) Matrix showing the fraction of reconstructed molecules that could be assigned to either one or N number of isoforms, where molecules were first grouped by the number of annotated isoform available for its genes. (f) Matrix showing the fraction of reconstructed molecules that could be assigned to either one or N number of isoforms (as in e) after we filtered the assignments to only those isoforms with detectable expression (TPM>0) in Salmon (including internal reads without linked UMIs). (g) Barplots showing the fraction of molecules assigned to different PTPRC isoforms, separated by cell type and aggregating over all cells within cell types. (h) Sashimi plots of reconstructed molecules assigned to either the RO or RABC isoform of PTPRC in gamma-delta T-cells. (i) Barplots showing the fraction of molecules assigned to different TIMP1 isoforms, separating by cell type and aggregating over cells within cell types. (j) Sashimi plots of reconstructed molecules assigned to two TIMP1 isoforms in FCGR3A+ monocytes.

FIGS. 18a & 18b. Mapping statistics of used Smart-seq2 and Smart-seq3 libraries. (FIG. 18a) Percentage of unmapped read pairs, and read pairs that aligned to exonic, intronic and intergenic regions. Separated per protocol (Smart-seq2 and Smart-seq3) and experiment (HEK293FT, Mouse Fibroblasts, HCA cells). (FIG. 18b) Mapping statistics for 5′UMI-containing read pairs in Smart-seq3. Percentage of unmapped read pairs, and read pairs that aligned to exonic, intronic and intergenic regions. Separated per experiment (HEK293FT, Mouse Fibroblasts, HCA cells).

FIG. 19 illustrates a method of producing 5′UMI reads and internals reads, following by construction of the full length sequence of an RNA therefrom, in accordance with an embodiment of the invention.

### DEFINITIONS

A barcode is a region that serves as an identifier of a nucleic acid. Barcodes may vary, wherein examples include RNA source barcodes, e.g., cell barcodes, host barcodes, etc.; container barcodes, such as plate or well barcodes; in-line barcodes, indexing barcodes, etc.

Unique Molecular Identifiers (i.e., UMIs) are randomers of varying length, e.g., ranging in length in some instances from 6 to 12 nts, that can be used for counting of individual molecules of a given molecular species. Counting is achieved by attaching UMIs from a diverse pool of UMIs to individual molecules of a target of interest such that each individual molecule receives a unique UMI. By counting individual transcript molecules, PCR bias can be reduced during NGS library prep and a more quantitative understanding of the sample population can be achieved. See e.g., U.S. Pat. No. 8,835,358; Fu et al., “Molecular Indexing Enables Quantitative Targeted RNA Sequencing and Reveals Poor Efficiencies in Standard Library Preparations,” PNAS (2014) 5: 1891-1896 and Fu et al., “Digital Encoding of Cellular mRNAs Enabling Precise and Absolute Gene Expression Measurement by Single-Molecule Counting,” Anal. Chem (2014) 86:2867-2870.

The term “complementary” as used herein refers to a nucleotide sequence that base-pairs by non-covalent bonds to all or a region of a target nucleic acid (e.g., a template RNA or other region of the double stranded product nucleic acid). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%). The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=#of identical positions/total #of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

As used herein, the term “hybridization conditions” means conditions in which a primer specifically hybridizes to a region of the target nucleic acid (e.g., a template RNA or other region of the double stranded product nucleic acid). Whether a primer specifically hybridizes to a target nucleic acid is determined by such factors as the degree of complementarity between the polymer and the target nucleic acid and the temperature at which the hybridization occurs, which may be informed by the melting temperature (T_{M}) of the primer. The melting temperature refers to the temperature at which half of the primer-target nucleic acid duplexes remain hybridized and half of the duplexes dissociate into single strands. The Tₘ of a duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., Ch. 10). Other more advanced models that depend on various parameters may also be used to predict Tₘ of primer/target duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

Next generation sequencing (NGS) libraries are libraries whose nucleic acid members include a partial or complete sequencing platform adapter sequence at their termini useful for sequencing using a sequencing platform of interest. Sequencing platforms of interest include, but are not limited to, the HiSeg™, MiSeq™ and Genome Analyzer™ sequencing systems from Illumine®, the Ion PGM™ and Ion Proton™ sequencing systems from Ion Torrent™; the PACBIO RS II Sequel system from Pacific Biosciences, the SOLiD sequencing systems from Life Technologies™, the 454 GS FLX+ and GS Junior sequencing systems from Roche, the MinION™ system from Oxford Nanopore, or any other sequencing platform of interest.

By “under conditions suitable for extension of the cDNA” is meant reaction conditions that permit polymerase-mediated extension of a 3′ end of the first strand cDNA primer hybridized to the template RNA, template switching of the polymerase to the template switch oligonucleotide (TSO), and continuation of the extension reaction using the template switch oligonucleotide as the template. Achieving suitable reaction conditions may include selecting reaction mixture components, concentrations thereof, and a reaction temperature to create an environment in which the polymerase is active and the relevant nucleic acids in the reaction interact (e.g., hybridize) with one another in the desired manner. For example, in addition to the template RNA, the polymerase, the first strand cDNA primer, the template switch oligonucleotide and dNTPs, the reaction mixture may include buffer components that establish an appropriate pH, salt concentration (e.g., KCl concentration), metal cofactor concentration (e.g., Mg²⁺ or Mn²⁺ concentration), and the like, for the extension reaction and template switching to occur. Other components may be included, such as one or more nuclease inhibitors (e.g., an RNase inhibitor and/or a DNase inhibitor), one or more additives for facilitating amplification/replication of GC rich sequences (e.g., GC-Mel™ reagent (Takara Bio USA, Inc. (Mountain View, Calif.)), betaine, DMSO, ethylene glycol, 1,2-propanediol, or combinations thereof), one or more molecular crowding agents (e.g., polyethylene glycol, Ficoll, dextran, or the like), one or more enzyme-stabilizing components (e.g., DTT, or TCEP, present at a final concentration ranging from 1 to 10 mM (e.g., 5 mM)), and/or any other reaction mixture components useful for facilitating polymerase-mediated extension reactions and template-switching.

The reaction mixture can have a pH suitable for the primer extension reaction and template-switching. In certain embodiments, the pH of the reaction mixture ranges from 5 to 9, such as from 7 to 9, including from 8 to 9, e.g., 8 to 8.5. In some instances, the reaction mixture includes a pH adjusting agent. pH adjusting agents of interest include, but are not limited to, sodium hydroxide, hydrochloric acid, phosphoric acid buffer solution, citric acid buffer solution, and the like. For example, the pH of the reaction mixture can be adjusted to the desired range by adding an appropriate amount of the pH adjusting agent.

The temperature range suitable for extension of the cDNA may vary according to factors such as the particular polymerase employed, the melting temperatures of any optional primers employed, etc. According to one embodiment, the reaction mixture conditions include bringing the reaction mixture to a temperature ranging from 4° C. to 72° C., such as from 16° C. to 70° C., e.g., 37° C. to 50° C., such as 40° C. to 45° C., including 42° C.

The template ribonucleic acid (RNA) molecule within the RNA sample may be a polymer of any length composed of ribonucleotides, e.g., 10 nts or longer, 20 nts or longer, 50 nts or longer, 100 nts or longer, 500 nts or longer, 1000 nts or longer, 2000 nts or longer, 3000 nts or longer, 4000 nts or longer, 5000 nts or longer or more nts. In certain aspects, the template ribonucleic acid (RNA) is a polymer composed of ribonucleotides, e.g., 10 nts or less, nts or less, 50 nts or less, 100 nts or less, 500 nts or less, 1000 nts or less, 2000 nts or less, 3000 nts or less, 4000 nts or less, or 5000 nts or less, 10,000 nts or less, 25,000 nts or less, 50,000 nts or less, 75,000 nts or less, 100,000 nts or less. The template RNA may be any type of RNA (or sub-type thereof) including, but not limited to, a messenger RNA (mRNA), a microRNA (miRNA), a small interfering RNA (siRNA), a transacting small interfering RNA (ta-siRNA), a natural small interfering RNA (nat-siRNA), a ribosomal RNA (rRNA), a transfer RNA (tRNA), a small nucleolar RNA (snoRNA), a small nuclear RNA (snRNA), a long non-coding RNA (IncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared siRNA (esiRNA), a small temporal RNA (stRNA), a signal recognition RNA, a telomere RNA, a ribozyme, a viral RNA or any combination of RNA types thereof or subtypes thereof.

The RNA sample that includes the template RNA may be combined into the reaction mixture in an amount sufficient for producing the product nucleic acid. According to one embodiment, the RNA sample is combined into the reaction mixture such that the final concentration of RNA in the reaction mixture is from 1 fg/μL to 10 μg/μL, such as from 1 μg/μL to 5 μg/μL, such as from 0.001 μg/μL to 2.5 μg/μL, such as from 0.005 μg/μL to 1 μg/μL, such as from 0.01 μg/μL to 0.5 μg/μL, including from 0.1 μg/μL to 0.25 μg/μL. In certain aspects, the RNA sample that includes the template RNA is isolated from a single cell. In other aspects, the RNA sample that includes the template RNA is isolated from 2, 3, 4, 5, 6, 7, 8, 9, 10 or more, 20 or more, 50 or more, 100 or more, or 500 or more cells, such as 750 or more cells, 1,000 or more cells, 2,000 or more cells, including 5,000 or more cells. In some instances, the RNA sample may be prepared from a tissue sample. According to certain embodiments, the RNA sample that includes the template RNA is isolated from 500 or less, 100 or less, 50 or less, 20 or less, 10 or less, 9, 8, 7, 6, 5, 4, 3, or 2 cells.

The template RNA may be present in any nucleic acid sample of interest, including but not limited to, a nucleic acid sample isolated from a single cell, a plurality of cells (e.g., cultured cells), a tissue, an organ, or an organism (e.g., bacteria, yeast, or higher eukaryotic organisms, such as a plant, or a mouse, or a worm, or the like). In certain aspects, the nucleic acid sample is isolated from a cell(s), tissue, organ, and/or the like, including but not limited to: embryos, blastocysts, spent media from embryo culture or other cell, tissue, or organ culture media. In other aspects, the sample may be isolated from a bodily compartment suitable for use in diagnosis, such as blood, urine, saliva, platelets, microvesicles, exosomes, serum, or other bodily fluids. In some aspects, the initial nucleic acid sample is obtained from a mammal (e.g., a human, a rodent (e.g., a mouse), or any other mammal of interest). In other aspects, the nucleic acid sample is isolated from a source other than a mammal, such as bacteria, yeast, insects (e.g., drosophila), amphibians (e.g., frogs (e.g., Xenopus)), viruses, plants, or any other non-mammalian nucleic acid sample source. Approaches, reagents and kits for isolating RNA from such sources are known in the art. For example, kits for isolating RNA from a source of interest—such as the NucleoSpin®, NucleoMag® and NucleoBond® RNA isolation kits by Clontech Laboratories, Inc. (Mountain View, Calif.)—are commercially available. In certain aspects, the RNA is isolated from a fixed biological sample, e.g., formalin-fixed, paraffin-embedded (FFPE) tissue. RNA from FFPE tissue may be isolated using commercially available kits—such as the NucleoSpin® FFPE RNA kits by Clontech Laboratories, Inc. (Mountain View, Calif.).

A variety of polymerases may be employed when practicing the subject methods. The polymerase combined into the reaction mixture in the template switching reaction is capable of template switching, where the polymerase uses a first nucleic acid strand as a template for polymerization, and then switches to the 3′ end of a second “acceptor” template nucleic acid strand to continue the same polymerization reaction (e.g., template switching). In certain aspects, the polymerase combined into the reaction mixture is a reverse transcriptase (RT). Reverse transcriptases capable of template-switching that find use in practicing the methods include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants, derivatives, or functional fragments thereof, e.g., RNase H minus or RNase H reduced enzymes (e.g. Superscript RT or Maxima H minus RT (Thermo Fisher)). For example, the reverse transcriptase may be a Moloney Murine Leukemia Virus reverse transcriptase (MMLV RT) or a Bombyx mori reverse transcriptase (e.g., Bombyx mori R2 non-LTR element reverse transcriptase). Polymerases capable of template switching that find use in practicing the subject methods are commercially available and include SMARTScribe™ reverse transcriptase available from Takara Bio USA, Inc. (Mountain View, Calif.). In certain aspects, a mix of two or more different polymerases is added to the reaction mixture, e.g., for improved processivity, proof-reading, and/or the like. In some instances, the polymer is one that is heterologous relative to the template, or source thereof. The polymerase is combined into the reaction mixture such that the final concentration of the polymerase is sufficient to produce a desired amount of the product nucleic acid. In certain aspects, the polymerase (e.g., a reverse transcriptase such as an MMLV RT or a Bombyx mori RT) is present in the reaction mixture at a final concentration of from 0.1 to 200 units/μL (U/μL), such as from 0.5 to 100 U/μL, such as from 1 to 50 U/μL, including from 5 to 25 U/μL, e.g., 20 U/μL.

In addition to a template switching capability, the polymerase combined into the reaction mixture may include other useful functionalities to facilitate production of the product nucleic acid. For example, the polymerase may have terminal transferase activity, where the polymerase is capable of catalyzing template-independent addition of deoxyribonucleotides to the 3′ hydroxyl terminus of a DNA molecule. In certain aspects, when the polymerase reaches the 5′ end of a template RNA, the polymerase is capable of incorporating one or more additional nucleotides at the 3′ end of the nascent strand not encoded by the template. For example, when the polymerase has terminal transferase activity, the polymerase may be capable of incorporating 1, 2, 3, 4, 5, 6, 7, 8, 9, 10 or more additional nucleotides at the 3′ end of the nascent DNA strand. In certain aspects, a polymerase having terminal transferase activity incorporates 10 or less, such as 5 or less (e.g., 3) additional nucleotides at the 3′ end of the nascent DNA strand. All of the nucleotides may be the same (e.g., creating a homonucleotide stretch at the 3′ end of the nascent strand) or at least one of the nucleotides may be different from the other(s). In certain aspects, the terminal transferase activity of the polymerase results in the addition of a homonucleotide stretch of 2, 3, 4, 5, 6, 7, 8, 9, 10 or more of the same nucleotides (e.g., all dCTP, all dGTP, all dATP, or all dTTP). According to certain embodiments, the terminal transferase activity of the polymerase results in the addition of a homonucleotide stretch of 10 or less, such as 9, 8, 7, 6, 5, 4, 3, or 2 (e.g., 3) of the same nucleotides. For example, according to one embodiment, the polymerase is an MMLV reverse transcriptase (MMLV RT). MMLV RT incorporates additional nucleotides (predominantly dCTP, e.g., three dCTPs) at the 3′ end of the nascent DNA strand. As described in greater detail elsewhere herein, these additional nucleotides may be useful for enabling hybridization between the 3′ end of the template switch oligonucleotide and the 3′ end of the nascent DNA strand, e.g., to facilitate template switching by the polymerase from the template RNA to the template switch oligonucleotide. For example, when a homonucleotide stretch is added to the nascent cDNA strand, the template switch oligonucleotide may have a 3′ hybridization domain complementary to the homonucleotide stretch to enable hybridization between the 3′ end of the template switch oligonucleotide and the 3′ end of the nascent cDNA strand. Similarly, when a heteronucleotide stretch is added to the nascent cDNA strand, the template switch oligonucleotide may have a 3′ hybridization domain complementary to the heteronucleotide stretch to enable hybridization between the 3′ end of the template switch oligonucleotide and the 3′ end of the nascent cDNA strand.

A cDNA synthesis primer is a primer that primes synthesis of a first strand cDNA using an RNA as a template. According to certain embodiments, the cDNA synthesis primer includes two or more domains. For example, the primer may include a first (e.g., 3′) domain that hybridizes to the template RNA and a second (e.g., 5′) domain that does not hybridize to the template RNA. The sequence of the first and second domains may be independently defined or arbitrary. In certain aspects, the first domain has a defined sequence (e.g., an oligo dT sequence or an RNA specific sequence) or an arbitrary sequence (e.g., a random sequence, such as a random hexamer sequence) and the sequence of the second domain is defined, e.g., an amplification primer site, such as PCR primer site, e.g., a reverse amplification primer site. In embodiments, the amplification primer site may the same or different as the amplification primer site of the template switch oligonucleotide.

By “sequencing platform adapter construct” is meant a nucleic acid construct that includes at least a portion of a nucleic acid domain (e.g., a sequencing platform adapter nucleic acid sequence) utilized by a sequencing platform of interest, such as a sequencing platform provided by Illumina® (e.g., the HiSeg™, MiSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems); or any other sequencing platform of interest. In certain aspects, a sequencing platform adapter construct includes one or more nucleic acid domains selected from: a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind); a barcode domain (e.g., a domain that uniquely identifies the sample source of the nucleic acid being sequenced to enable sample multiplexing by marking every molecule from a given sample with a specific barcode or “tag”); a barcode sequencing primer binding domain (a domain to which a primer used for sequencing a barcode binds); a molecular identification domain (e.g., a molecular index tag, such as a randomized tag of 4, 6, or other number of nucleotides) for uniquely marking molecules of interest to determine expression levels based on the number of instances a unique tag is sequenced; or any combination of such domains. In certain aspects, a barcode domain (e.g., sample index tag) and a molecular identification domain (e.g., a molecular index tag) may be included in the same nucleic acid. A sequencing platform adapter domain, when present, may include one or more nucleic acid domains of any length and sequence suitable for the sequencing platform of interest. In certain aspects, the nucleic acid domains are from 4 to 200 nts in length. For example, the nucleic acid domains may be from 4 to 100 nts in length, such as from 6 to 75, from 8 to 50, or from to 40 nts in length. According to certain embodiments, the sequencing platform adapter construct includes a nucleic acid domain that is from 2 to 8 nucleotides in length, such as from 9 to 15, from 16 to 22, from 23 to 29, or from 30 to 36 nts in length.

The nucleic acid domains may have a length and sequence that enables a polynucleotide (e.g., an oligonucleotide) employed by the sequencing platform of interest to specifically bind to the nucleic acid domain, e.g., for solid phase amplification and/or sequencing by synthesis of the cDNA insert flanked by the nucleic acid domains. Example nucleic acid domains include the P5 (5′-AATGATACGGCGACCACCGA-3′)(SEQ ID NO:01), P7 (5′-CAAGCAGAAGACGGCATACGAGAT-3′)(SEQ ID NO:02), Read 1 primer (5′-ACACTCTTTCCCTACACGACGCTCTTCCGATCT-3′)(SEQ ID NO:03) and Read 2 primer (5′-GTGACTGGAGTTCAGACGTGTGCTCTTCCGATCT-3′)(SEQ ID NO:04) domains employed on the Illumina®-based sequencing platforms. Other example nucleic acid domains include the A adapter (5′-CCATCTCATCCCTGCGTGTCTCCGACTCAG-3′)(SEQ ID NO:05) and P1 adapter (5′-CCTCTCTATGGGCAGTCGGTGAT-3′)(SEQ ID NO:06) domains employed on the Ion Torren™-based sequencing platforms. The nucleotide sequences of nucleic acid domains useful for sequencing on a sequencing platform of interest may vary and/or change over time. Adapter sequences are typically provided by the manufacturer of the sequencing platform (e.g., in technical documents provided with the sequencing system and/or available on the manufacturer's website). Based on such information, the sequence of any sequencing platform adapter domains of the template switch oligonucleotide, first strand cDNA primer, amplification primers, and/or the like, may be designed to include all or a portion of one or more nucleic acid domains in a configuration that enables sequencing the nucleic acid insert (corresponding to the template RNA) on the platform of interest.

The cDNA synthesis primer may include one or more nucleotides (or analogs thereof) that are modified or otherwise non-naturally occurring. For example, the primer may include one or more nucleotide analogs (e.g., LNA, FANA, 2′-O-Me RNA, 2′-fluoro RNA, or the like), linkage modifications (e.g., phosphorothioates, 3′-3′ and 5′-5′ reversed linkages), 5′ and/or 3′ end modifications (e.g., 5′ and/or 3′ amino, biotin, DIG, phosphate, thiol, dyes, quenchers, etc.), one or more fluorescently labeled nucleotides, or any other feature that provides a desired functionality to the primer that primes cDNA synthesis.

In embodiments, it may be desirable to prevent any subsequent extension reactions which use the double stranded product nucleic acid as a template from extending beyond a particular position in the region of the double stranded product nucleic acid corresponding to the primer. For example, according to certain embodiments, the first strand cDNA primer includes a polymerase blocking modification that prevents a polymerase using the region corresponding to the primer as a template from polymerizing a nascent strand beyond the modification. Useful modifications include, but are not limited to, an abasic lesion (e.g., a tetrahydrofuran derivative), a nucleotide adduct, an iso-nucleotide base (e.g., isocytosine, isoguanine, and/or the like), and any combination thereof. Such blocking modifications may be included in any of the nucleic acid reagents used when practicing the methods of the present disclosure, including first strand cDNA primer, the template switch oligonucleotide, first and second amplification, e.g., PCR, primers used for amplifying the first-strand cDNA to produce the product double stranded cDNA, amplification primers used for PCR amplification of tagmentation products, and any combination thereof. In some instances, primers employed in methods of the invention, such as amplification, e.g., PCR, primers, include a ligation block. Ligation blocks of interest that may be present in a given primer, as desired, include but are not limited to: amine, inverted T, and Biotin-TEG.

By “template switch oligonucleotide” is meant an oligonucleotide template to which a polymerase switches from an initial template (e.g., a template RNA) during a nucleic acid polymerization reaction. In this regard, a template RNA may be referred to as a “donor template” and the template switch oligonucleotide may be referred to as an “acceptor template.” As used herein, an “oligonucleotide” can refer to a single-stranded multimer of nucleotides from 2 to 500 nts, e.g., 2 to 200 nts. Oligonucleotides may be synthetic or may be made enzymatically, and, in some embodiments, are 10 to 50 nts in length. Oligonucleotides may contain ribonucleotide monomers (i.e., may be oligoribonucleotides or “RNA oligonucleotides”) or deoxyribonucleotide monomers (i.e., may be oligodeoxyribonucleotides or “DNA oligonucleotides”). Oligonucleotides may be 10 to 20, 21 to 30, 31 to 40, 41 to 50, 51 to 60, 61 to 70, 71 to 80, 80 to 100, 100 to 150 or 150 to 200, up to 500 or more nts in length, for example. When employed, in some instances the template switch oligonucleotide may be added to the reaction mixture at a final concentration of from 0.01 to 100 μM, such as from 0.1 to 10 μM, such as from 0.5 to 5 μM, including 2 to 3 μM.

The template switch oligonucleotide may include one or more nts (or analogs thereof) that are modified or otherwise non-naturally occurring. For example, the template switch oligonucleotide may include one or more nucleotide analogs (e.g., LNA, FANA, 2′-O-Me RNA, 2′-fluoro RNA, or the like), linkage modifications (e.g., phosphorothioates, 3-3′ and 5-5′ reversed linkages), 5′ and/or 3′ end modifications (e.g., 5′ and/or 3′ amino, biotin, DIG, phosphate, thiol, dyes, quenchers, etc.), one or more fluorescently labeled nts, or any other feature that provides a desired functionality to the template switch oligonucleotide. Any desired nucleotide analogs, linkage modifications and/or end modifications may be included in any of the nucleic acid reagents used when practicing the methods of the present disclosure.

The template switch oligonucleotide may include a 3′ hybridization domain and a 5′ amplification primer site. The 3′ hybridization domain may vary in length, and in some instances ranges from 2 to 10 nts in length, such as from 3 to 7 nts in length. The sequence of the 3′ hybridization domain, i.e., template switch domain, may be any convenient sequence, e.g., an arbitrary sequence, a heterpolymeric sequence (e.g., a hetero-trinucleotide) or homopolymeric sequence (e.g., a homo-trinucleotide, such as G-G-G), or the like. Examples of 3′ hybridization domains and template switch oligonucleotides are further described in U.S. Pat. No. 5,962,272 and published PCT application publication no. WO2015027135, the disclosures of which are herein incorporated by reference.

According to certain embodiments, the template switch oligonucleotide includes a modification that prevents the polymerase from switching from the template switch oligonucleotide to a different template nucleic acid after synthesizing the compliment of the 5′ end of the template switch oligonucleotide (e.g., a 5′ adapter sequence of the template switch oligonucleotide). Useful modifications include, but are not limited to, an abasic lesion (e.g., a tetrahydrofuran derivative), a nucleotide adduct, an iso-nucleotide base (e.g., isocytosine, isoguanine, and/or the like), and any combination thereof.

In addition to the above components, the template switch oligonucleotide may further include a number of additional components or domains positioned between the 5′ and 3′ domains described above, such as but not limited to: barcode domains, unique molecular identifier domains, a sequencing platform adapter construct domains, etc., where these domains may be as described above.

Fragmentation refers to any protocol in which nucleic acid molecules are disrupted into shorter fragments. Fragmentation protocols include, but are not limited to: moving an RNA sample one or more times through a micropipette tip or fine-gauge needle, nebulizing the sample, sonicating the sample (e.g., using a focused-ultrasonicator by Covaris, Inc. (Woburn, Mass.)), bead-mediated shearing, enzymatic shearing (e.g., using one or more RNA-shearing enzymes, or by enzymatic digestions, e.g., with restriction enzymes or other endonucleases appropriate for the polynucleotides of interest), chemical based fragmentation, e.g., using divalent cations, fragmentation buffer (which may be used in combination with heat) or any other suitable approach for shearing/fragmenting a precursor RNA to generate a shorter template RNA. In certain aspects, the nucleic acid fragments generated by fragmentation of a starting nucleic acid sample has a length of from 10 to 20 nts, from 20 to 30 nts, from 30 to 40 nts, from 40 to 50 nts, from 50 to 60 nts, from 60 to 70 nts, from 70 to 80 nts, from 80 to 90 nts, from 90 to 100 nts, from 100 to 150 nts, from 150 to 200 nts, from 200 to 250 nts in length, or from 200 to 1000 nts or even from 1000 to 10,000 nts in length, for example, as appropriate for the sequencing platform chosen.

In some instances, fragmentation comprises tagmentation, i.e., transposome mediated fragmentation. In transposome mediated fragmentation (tagmentation), transposomes are prepared with DNA that is afterwards cut so that the transposition events result in fragmented DNA with adapters (instead of an insertion). Transposomes employed in methods of the present disclosure include a transposase and a transposon nucleic acid that may include a transposon end domain among other domains. Any domains are defined functionally and so may be one in the same sequence or may be different sequences, as desired. The domains may also overlap.

A “transposase” means an enzyme that is capable of forming a functional complex with a transposon end domain-containing composition (e.g., transposons, transposon ends, transposon end compositions) and catalyzing insertion or transposition of the transposon end-containing composition into the double-stranded target DNA with which it is incubated in an in vitro transposition reaction. Transposases that find use in practicing the methods of the present disclosure include, but are not limited to, Tn5 transposases, Tn7 transposases, and Mu transposases. The transposase may be a wild-type transposase. In other aspects, the transposase includes one or more modifications (e.g., amino acid substitutions) to improve a property of the transposase, e.g., enhance the activity of the transposase. For example, hyperactive mutants of the Tn5 transposase having substitution mutations in the Tn5 protein (e.g., E54K, M56A and L372P) have been developed and are described in, e.g., Picelli et al. (2013) Genome Research 24:2033-2040. Additional Tn5 substitution mutations include, but are not limited to: Y41H; T47P; E54V, E110K, P242A, E344A, and E345A. A given Tn5 mutant may include one or more substitutions, where combinations of substitutions that may be present include, but are not limited to: T47P, M56A and L372P; TT47P, M56A, P242A and L372P; and M56A, E344A and L372P.

The term “transposon end domain” means a double-stranded DNA that includes the nucleotide sequences (the “transposon end sequences”) that are necessary to form the complex with the transposase or integrase enzyme that is functional in an in vitro transposition reaction. A transposon end domain forms a “complex” or a “synaptic complex” or a “transposome complex” or a “transposome composition” with a transposase or integrase that recognizes and binds to the transposon end domain, and which complex is capable of inserting or transposing the transposon end domain into target DNA with which it is incubated in an in vitro transposition reaction. A transposon end domain exhibits two complementary sequences consisting of a “transferred transposon end sequence” or “transferred strand” and a “non-transferred transposon end sequence,” or “non-transferred strand.” For example, one transposon end domain that forms a complex with a hyperactive Tn5 transposase (e.g., EZ-Tn5 Transposase, EPICENTRE Biotechnologies, Madison, Wis., USA) that is active in an in vitro transposition reaction includes a transferred strand that exhibits a “transferred transposon end sequence” as follows: 5′ AGATGTGTATAAGAGACAG 3′, (SEQ ID NO:07) and a non-transferred strand that exhibits a “non-transferred transposon end sequence” as follows: 5′ CTGTCTCTTATACACATCT 3′ (SEQ ID NO:8). The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction. The sequence of the particular transposon end domain to be employed when practicing the methods of the present disclosure will vary depending upon the particular transposase employed. For example, a Tn5 transposon end domain may be included in the transposon nucleic acid when used in conjunction with a Tn5 transposase.

In addition to the transposon end domain, the transposon nucleic acid may also include one or more additional domains, such as a post tagmentation amplification primer site. In some instances, the post-tagmentation amplification primer site includes a sequencing platform adapter construct domain, e.g., as described above. This domain may be a nucleic acid domain selected from a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system), a sequencing primer binding domain (e.g., a domain to which the Read 1 or Read 2 primers of the Illumina® platform may bind), a barcode domain (e.g., a domain that uniquely identifies the sample source of the nucleic acid being sequenced to enable sample multiplexing by marking every molecule from a given sample with a specific barcode or “tag”), a barcode sequencing primer binding domain (a domain to which a primer used for sequencing a barcode binds), a molecular identification domain, or any combination of such domains.

When it is desirable to prepare transposomes for the tagmentation step, any suitable transposome preparation approach may be used, and such approaches may vary depending upon, e.g., the specific transposase and transposon nucleic acids to be employed. For example, the transposon nucleic acids and transposase may be incubated together at a suitable molar ratio (e.g., a 2:1 molar ratio, a 1:1 molar ratio, a 1:2 molar ratio, or the like) in a suitable buffer. According to one embodiment, when the transposase is a Tn5 transposase, preparing transposomes may include incubating the transposase and transposon nucleic acid at a 1:1 molar ratio in 2× Tn5 dialysis buffer for a sufficient period of time, such as 1 hour.

Tagmenting includes contacting the double stranded nucleic acids with a transposome under tagmentation conditions. Such conditions may vary depending upon the particular transposase employed. In some instances, the conditions include incubating the transposomes and tagged extension products in a buffered reaction mixture (e.g., a reaction mixture buffered with Tris-acetate, or the like) at a pH of from 7 to 8, such as pH 7.5. The transposome may be provided such that about a molar equivalent, or a molar excess, of the transposon is present relative to the tagged extension products. Suitable temperatures include from 32° to 42° C., such as 37° C. The reaction is allowed to proceed for a sufficient amount of time, such as from 5 minutes to 3 hours. The reaction may be terminated by adding a solution (e.g., a “stop” solution), which may include an amount of SDS and/or other transposase reaction termination reagent suitable to terminate the reaction. Protocols and materials for achieving fragmentation of nucleic acids using transposomes are available and include, e.g., those provided in the EZ-Tn5™ transpose kits available from EPICENTRE Biotechnologies (Madison, Wis., USA).

In some aspects of the invention, the methods include the step of obtaining single cells. Obtaining single cells may be done according to any convenient protocol. A single cell suspension can be obtained using standard methods known in the art including, for example, enzymatically using trypsin or papain to digest proteins connecting cells in tissue samples or releasing adherent cells in culture, or mechanically separating cells in a sample. Single cells can be placed in any suitable reaction vessel in which single cells can be treated individually. For example a 96-well plate, 384 well plate, or a plate with any number of wells such as 2000, 4000, 6000, or 10000 or more. The multi-well plate can be part of a chip and/or device. The present disclosure, is not limited by the number of wells in the multi-well plate. In various embodiments, the total number of weds on the plate is from 100 to 200,000, or from 5000 to 10,000. In other embodiments the plate comprises smaller chips, each of which includes 5,000 to 20,000 wells. For example, a square chip may include 125 by 125 nanowells, with a diameter of 0.1 mm. The wells (e.g., nanowells) in the multi-well plates may be fabricated in any convenient size, shape or volume. The well may be 100 μm to 1 mm in length, 100 μm to 1 mm in width, and 100 μm to 1 mm in depth. In various embodiments, each nanowell has an aspect ratio (ratio of depth to width) of from 1 to 4. In one embodiment, each nanowell has an aspect ratio of 2. The transverse sectional area may be circular, elliptical, oval, conical, rectangular, triangular, polyhedral, or in any other shape. The transverse area at any given depth of the well may also vary in size and shape. In certain embodiments, the weds have a volume of from 0.1 nl to 1 μl. The nanowell may have a volume of 1 μl or less, such as 500 nl or less. The volume may be 200 nl or less, such as 100 nl or less. In an embodiment, the volume of the nanowell is 100 nl. Where desired, the nanowell can be fabricated to increase the surface area b volume ratio, thereby facilitating heat transfer through the unit, which can reduce the ramp time of a thermal cycle. The cavity of each well (e.g., nanowell) may take a variety of configurations. For instance, the cavity within a well may be divided by linear or curved walls to form separate but adjacent compartments, or by circular walls to form inner and outer annular compartments. The wells can be designed such that a single well includes a single cell. An individual cell may also be isolated in any other suitable container, e.g., microfluidic chamber, droplet, nanowell, tube, etc.—Any convenient method for manipulating single cells may be employed, where such methods include fluorescence activated cell sorting (FACS), robotic device injection, gravity flow, or micromanipulation and the use of semi-automated cell pickers (e.g. the Quixell™ cell transfer system from Stoelting Co.), etc. In some instances, single cells can be deposited in wells of a plate according to Poisson statistics (e.g., such that approximately 10%, 20%, 30% or 40% or more of the wells contain a single cell—which number can be defined by adjusting the number of cells in a given unit volume of fluid that is to be dispensed into the containers). In some instances, a suitable reaction vessel comprises a droplet (e.g., a microdroplet). Individual cells can, for example, be individually selected based on features detectable by microscopic observation, such as location, morphology, reporter gene expression, antibody labelling, FISH, intracellular RNA labelling, or qPCR.

Following obtainment of single cells, e.g., as described above, mRNA can be released from the cells by lysing the cells. Lysis can be achieved by, for example, heating or freeze-thaw of the cells, or by the use of detergents or other chemical methods, or by a combination of these. However, any suitable lysis method can be used. A mild lysis procedure can advantageously be used to prevent the release of nuclear chromatin, thereby avoiding genomic contamination of the cDNA library, and to minimize degradation of mRNA. For example, heating the cells at 72° C. for 2 minutes in the presence of Tween-20 is sufficient to lyse the cells while resulting in no detectable genomic contamination from nuclear chromatin. Alternatively, cells can be heated to 65° C. for 10 minutes in water (Esumi et al., Neurosci Res 60(4):439-51 (2008)); or 70° C. for 90 seconds in PCR buffer II (Applied Biosystems) supplemented with 0.5% NP-40 (Kurimoto et al., Nucleic Acids Res 34(5):e42 (2006)); or lysis can be achieved with a protease such as Proteinase K or by the use of chaotropic salts such as guanidine isothiocyanate (U.S. Publication No. 2007/0281313).

In certain embodiments of the methods described herein, cells are obtained from a tissue of interest and a single-cell suspension is obtained. A single cell is placed in one well of a multi-well plate, or other suitable container, such as a microfluidic chamber or tube. The cells are lysed and reverse transcription reaction mix is added directly to the lysates without additional purification. It is also possible that the container vessel also contains reverse transcription reagents when the cells are lysed. The NGS libraries produced according to the methods of the present disclosure may exhibit a desired complexity (e.g., high complexity). The “complexity” of a NGS library relates to the proportion of redundant sequencing reads (e.g., sharing identical start sites) obtained upon sequencing the library. Complexity is inversely related to the proportion of redundant sequencing reads. In a low complexity library, certain target sequences are over-represented, while other targets (e.g., mRNAs expressed at low levels) suffer from little or no coverage. In a high complexity library, the sequencing reads more closely track the known distribution of target nucleic acids in the starting nucleic acid sample, and will include coverage, e.g., for targets known to be present at relatively low levels in the starting sample (e.g., mRNAs expressed at low levels). According to certain embodiments, the complexity of a NGS library produced according to the methods of the present disclosure is such that sequencing reads are produced for 70% or more, 75% or more, 80% or more, 85% or more, 90% or more, 95% or more, 96% or more, 97% or more, 98% or more, or 99% or more of the different species of target nucleic acids (e.g., different species of mRNAs) in the starting nucleic acid sample (e.g., RNA sample). The complexity of a library may be determined by mapping the sequencing reads to a reference genome or transcriptome (e.g., for a particular cell type). Specific approaches for determining the complexity of sequencing libraries have been developed, including the approach described in Daley et al. (2013) Nature Methods 10(4):325-327.

In certain aspects, the methods of the present disclosure further include subjecting the NGS library to a NGS protocol. The protocol may be carried out on any suitable NGS sequencing platform. NGS sequencing platforms of interest include, but are not limited to, a sequencing platform provided by Illumina® (e.g., the HiSeg™, MiSeq™ and/or NextSeq™ sequencing systems); Ion Torrent™ (e.g., the Ion PGM™ and/or Ion Proton™ sequencing systems); Pacific Biosciences (e.g., the PACBIO RS II Sequel sequencing system); Life Technologies™ (e.g., a SOLiD sequencing system); Roche (e.g., the 454 GS FLX+ and/or GS Junior sequencing systems); or any other sequencing platform of interest. The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS sequencing system employed.

In certain embodiments, the subject methods may be used to generate a NGS library corresponding to mRNAs for downstream sequencing on a sequencing platform of interest (e.g., a sequencing platform provided by Illumina®, Ion Torrent™, Pacific Biosciences, Life Technologies™, Roche, or the like). According to certain embodiments, the subject methods may be used to generate a NGS library corresponding to non-polyadenylated RNAs for downstream sequencing on a sequencing platform of interest. For example, microRNAs may be polyadenylated and then used as templates in a template switch polymerization reaction as described elsewhere herein. Random or gene-specific priming may also be used, depending on the goal of the researcher. The library may be mixed 50:50 with a control library (e.g., Illumina®'s PhiX control library) and sequenced on the sequencing platform (e.g., an Illumina® sequencing system). The control library sequences may be removed and the remaining sequences mapped to the transcriptome of the source of the mRNAs (e.g., human, mouse, or any other mRNA source).

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### DETAILED DESCRIPTION

The present invention generally relates to complementary deoxyribonucleic acid (cDNA) synthesis, and in particular to method and kit for preparing cDNA suitable for sequencing.

Embodiments of the invention prepares cDNA molecules that are suitable for sequencing and, in some instances, useful in single cell ribonucleic acid sequencing (scRNA-seq) methods. Embodiments of the invention, in clear contrast to prior art scRNA-seq methods, achieve the benefits of both main methods, i.e., they are compatible with unique molecular identifier (UMIs) used to remove the biased amplification effect and thereby enable counting of RNA molecules present prior to amplification and provide up to full-length transcript coverage and capture a large fraction of the RNA molecules present in the cells. The prior art second main methods, including Smart-seq and Smart-seq2, provide the most sensitive information of single-cell transcriptomes but suffer from being incompatible with UMIs and can therefore not be used to count RNA molecules in single cells.

Embodiments of the invention therefore enable simultaneous counting of RNA molecules and full-length coverage of transcriptomes in single cells. Importantly, embodiments of the invention can be used to generate single cell cDNAs that contain both UMIs, for RNA molecule counting, as well as full-transcript read coverage. Embodiments of the invention also enable paired-end sequencing of both internal fragments and 5′ end fragments, thus enabling better mapping of the fragments and a more detailed assessment of the structure of the template RNA from which the fragments were derived, such as transcript isoforms, SNP phasing, etc. Embodiments of the invention additionally enable biochemically fine-tuning the percentage of UMI-containing 5′ reads within the final sequencing library. This ability makes embodiments of the invention, also referred as Smart-seq3 herein, not only the most sensitive method to date, but also flexible and adaptable to different experimental needs.

In an embodiment, the method is based on hybridization of an oligo-dT that harbors a primer site, such as a reverse amplification primer site, to the poly-A tail of an RNA molecule, e.g., an mRNA of an RNA sample. A reverse transcriptase (RT) enzyme polymerizes cDNA using the full length of the RNA molecule as a template. When the RT reaches to the end of the RNA molecule, the polymerization is preferably still continued without any template by adding a few nucleotides to the 3′ end of the cDNA strand. A template switching oligonucleotide (TSO) harboring another primer site, such as a partial TN5 motif primers site, a novel identification tag, UMI and three rGs, hybridizes to the non-templated nucleotides at the 3′ end of the cDNA strand. RT continues the polymerization using the TSO as a new template to get an extended cDNA strand that has a respective primer site at both ends. In some embodiments, usage of additional free ribonucleotides, dCTPs or PEG enable increased efficiency of the template switching reaction in terms of genes captured.

In an embodiment, the extended cDNA strand is amplified using two primers in a PCR reaction and the amplified product is, in some instances, fragmented using, for instance, ILLUMINA® Nextera XT kit to be prepared for sequencing by ILLUMINA® platforms. The identification tag and UMI in the TSO are designed to be read by ILLUMINA® sequencers independent of the tagmentation and fragmentation reaction in the ILLUMINA® Nextera kit. Therefore, after sequencing, the reads that belong to the 5′ end of RNA molecules can be captured by recognition of the identification tag and can be quantified based on the UMI in order to calculate the number of unique RNA molecules observed. Simultaneously, the remaining internal reads can be used to map full-length transcript features, including exons, introns and genetic variation within transcribed parts of the genome.

The present invention has the unique capability to combine UMI-based RNA counting with full-length transcript coverage and paired-end sequencing. Experimental data as presented herein show that the invention provides the most sensitive profiling of RNA molecules from single cells, i.e. the generated sequencing libraries contain fragments from larger fractions of RNAs in cells than all previous methods.

The invention uses a template switching oligonucleotide (TSO) that enables the construction of 5′ tagged and full-length RNA fragments in the same sequencing library. The TSO is designed to comprise a primer site for PCR amplification, a unique identification tag that can identify 5′ reads from complex mixtures, a UMI, and multiple predefined nucleotides, such as three rGs, to anneal to the extended and non-templated bases on the cDNA strand.

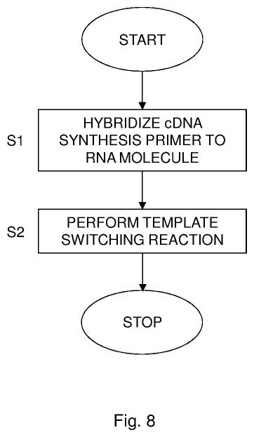

Hence, an aspect of the invention relates to a method for preparing cDNA, see FIG. 8. The method comprises hybridizing, in step S1, a cDNA synthesis primer to an RNA molecule and synthesizing a cDNA strand complementary to at least a portion of the RNA molecule to form an RNA-cDNA intermediate, sometimes also referred as an RNA-cDNA duplex. The method also comprises step S2, which comprises performing a template switching reaction by contacting the RNA-cDNA intermediate with a template switching oligonucleotide (TSO) under conditions suitable for extension of the cDNA strand using the TSO as template to form an extended cDNA strand. The extended cDNA strand is complementary to the at least a portion of the RNA molecule and the TSO. According to the invention, the TSO comprises an amplification primer site, an identification tag, a UMI and multiple predefined nucleotides.

The two steps S1 and S2 in FIG. 8 may be performed serially, i.e., step S1 prior to step S2. In such a case, the TSO is added, in step S2, to the reaction mixture from step S1. It is, however, alternatively possible to perform the two steps S1 and S2 together in a single reaction step. In such a case, the TSO and the cDNA synthesis primer is present in the reaction mixture together with the RNA molecule to synthesize the cDNA strand and form the RNA-cDNA intermediate and extend the cDNA strand into the extended cDNA strand.

The product of the method steps S1 and S2 shown in FIG. 8 is therefore an extended cDNA strand. This extended cDNA strand is complementary to at least a portion of the RNA molecule, such as the full RNA molecule, and is also complementary to the TSO. This means that the extended cDNA strand comprises a DNA sequence that is complementary to the at least a portion of the RNA molecule and a DNA sequence that is complementary to the TSO. This latter complementary DNA sequence therefore comprises a first subsequence that is complementary to the amplification primer site of the TSO, a second subsequence that is complementary to the identification tag, a third subsequence that is complementary to the UMI and a fourth subsequence that is complementary to the multiple, i.e., more than one, predefined nucleotides.

In an embodiment, step S1 of FIG. 8 comprises hybridizing the cDNA synthesis primer to the RNA molecule and synthesizing the cDNA strand by reverse transcription to form the RNA-cDNA intermediate. In this embodiment, step S2 comprises performing the template switching reaction by contacting the RNA-cDNA intermediate with the TSO under conditions suitable for extension of the cDNA strand by reverse transcription to form the extended cDNA strand.

Hence, reverse transcription is preferably used to synthesize the cDNA strand in step S1 and also used in step S2 to extend the cDNA strand into the extended cDNA strand. In an embodiment, a same reverse transcriptase could be used in the reverse transcription reaction in step S1 as in step S2. It is, however, possible to use a first reverse transcriptase in step S1 and then a second reverse transcriptase in step S2.

As reviewed above, illustrative, but non-limiting, examples of reverse transcriptases that can be used according to the embodiments include a human immunodeficiency virus type 1 (HIV-1) reverse transcriptase, a Moloney murine leukemia virus (M-MLV) reverse transcriptase, an avian myeloblastosis virus (AMV) reverse transcriptase, a telomerase reverse transcriptase and a mutated or genetically engineered version thereof. For instance, the reverse transcriptase is preferably a M-MLV reverse transcriptase and is more preferably selected from the group consisting of SuperScript™ II reverse transcriptase, SuperScript™ III reverse transcriptase, SuperScript™ IV reverse transcriptase, RevertAid H Minus reverse transcriptase, ProtoScript® II reverse transcriptase, Maxima H Minus reverse transcriptase and EpiScript™ reverse transcriptase. In a particular embodiment, the reverse transcriptase used in steps S1 and S2 is Maxima H Minus reverse transcriptase. Maxima H Minus reverse transcriptase is thermostable and has high processivity. Hence, this particular reverse transcriptase enables conducting the reverse transcription at elevated temperatures, i.e., above 37° C., and during shorter reaction times.

In an embodiment, the reverse transcription in steps S1 and S2 is conducted in the presence of ribonucleotides, including guanine ribonucleotides. In such an embodiment, the ribonucleotides are present at a concentration selected within an interval of from 0.05 mM to 10 mM, preferably within an interval of from 0.1 mM to 3 mM, such as about 1 mM. The addition of complementary ribonucleotides to the template switching reaction promotes longer and more stable non-templated C-tails in the context of M-MLV reverse transcriptase when the reverse transcriptase reaches the 5′ end of the RNA molecule acting as template. Such complementary ribonucleotides can also be used to fine tune the efficiency of the template switching reaction. Experimental data as presented herein show that addition of guanine ribonucleotides can be used to control gene capture and control the fraction of 5′ reads in the resulting sequencing library.

In an embodiment, the reverse transcription is conducted in the presence of a mixture dATP, dGTP, dTTP and dCTP. The mixture preferably comprises a same concentration of dATP, dGTP and dTTP and a concentration of dCTP is X mM higher than the same concentration of dATP, dGTP and dTTP. Hence, if the concentration of each of dATP, dGTP and dTTP in the mixture is Y mM then the concentration of dCTP in the mixture is preferably X+Y mM. In an embodiment, X is selected within an interval of from 0.05 mM to 10 mM, preferably within an interval of from 0.1 mM to 3 mM, such as about 1 mM. In an embodiment, Y is selected within an interval of from 0.05 mM to 10 mM, preferably within an interval of from 0.1 mM to 3 mM, such as about 0.5 mM.

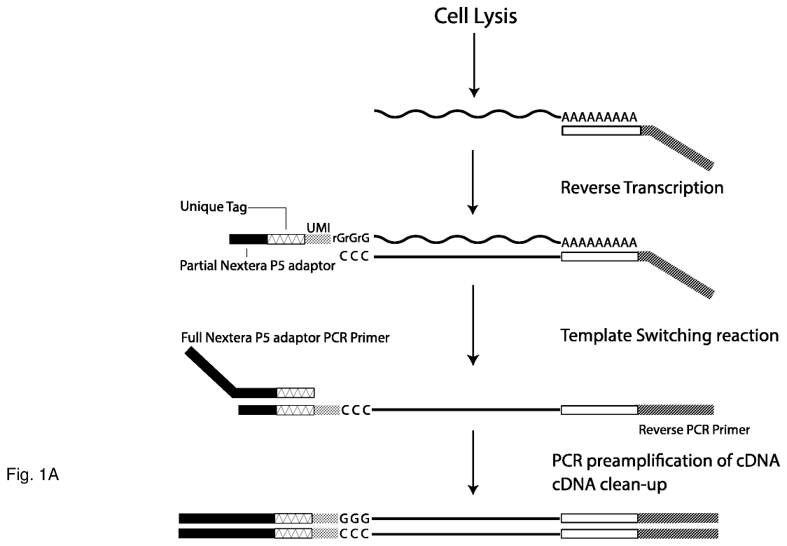

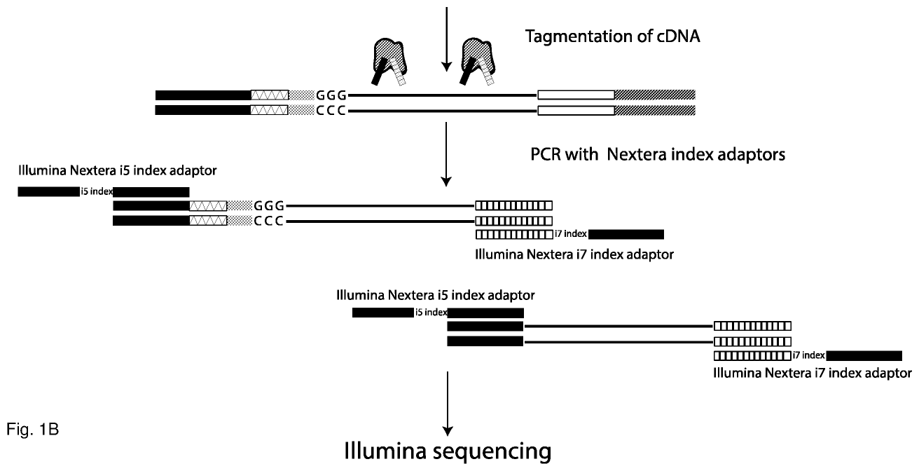

The deoxynucleotides (dNTPs) are used in the reverse transcription in order to synthesize and extend the cDNA strand. Extra dCTP is preferably added to the reverse transcription and template switching reaction to increase C incorporation into a non-templated stretch of nucleotides at the 3′ end of the cDNA strand. Hence, the 3′ end of the synthesized cDNA strand preferably comprises a stretch of Cs as schematically illustrated in FIG. 1A. In such a case, the multiple predefined nucleotides are preferably guanine nucleotides, such as guanine ribonucleotides (rG), guanine deoxynucleotides (dG), locked nucleic acid (LNA) guanine (LNA-G), 2′-fluoro-guanine (fG) and any combination thereof. The multiple predefined nucleotides of the TSO are thereby preferably complementary to the non-templated stretch of nucleotides added to the 3′ end of the cDNA strand in the reverse transcription performed in step S1.

The particular ribonucleotides present in the reverse transcription are preferably the same nucleobase as the multiple predefined nucleotides of the TSO. Furthermore, the extra nucleotides present in the reverse transcription are preferably complementary to this nucleobase. This means that other combinations of nucleobases than G and C could be used. For instance, the multiple predefined nucleotides could be multiple guanine nucleotides, multiple cytosine nucleotides, multiple adenine nucleotides or multiple thymidine nucleotides. The added ribonucleotides are then guanine ribonucleotides, cytosine ribonucleotides, adenine ribonucleotides or uracil ribonucleotides and the extra nucleotides are dCTP, dGTP, dTTP or dATP.

In an embodiment, the reverse transcription is conducted in the presence of a magnesium salt in a concentration selected within an interval of from 0.1 mM to 20 mM, preferably within an interval of from 1 mM to 10 mM, and more preferably within an interval of from 2 mM to 5 mM, such as about 3 mM. In an embodiment, the magnesium salt is selected from the group consisting of MgCl₂, MgOAc and MgSO₂. In a preferred embodiment, the magnesium salt is MgCl₂. The comparatively low concentration of the magnesium salt in the reverse transcription reduces the fidelity of the reverse transcriptase.

In an embodiment, the reverse transcription is conducted in the presence of a chloride salt selected from the group consisting of sodium chloride (NaCl), cesium chloride (CsCl), and a mixture thereof. The chloride salt is preferably present in a concentration selected within an interval of from 5 mM to 500 mM, preferably within an interval of from 15 mM to 250 mM, and more preferably within an interval of from 25 mM to 150 mM, such as from 50 mM to 100 mM, or about 75 mM.

In an embodiment, the reverse transcription is conducted in an at least reduced amount, if not the absence of, potassium chloride (KCl). KCl promotes a four-stranded structure in the RNA molecule when there is a stretch of rG nucleotides, either intramolecularly or intermolecularly. The structure is called G-quadruplex and inhibits the reverse transcription reaction. Using a chloride salt other than KCl improves the reverse transcription reaction, likely be lowering the appearance of G-quadruplex RNA secondary structures. Both NaCl and CsCl resulted in higher reverse transcription efficiency as compared to KCl with Maxima H Minus reverse transcriptase.

In an embodiment, at least one reverse transcription and/or amplification enhancer is added to promote enzymatic reaction rates of the reverse transcription and/or amplification reaction. Non-limiting, but illustrative, examples of such enhances include betaine, bovine serum albumin (BSA), glycerol, polyethylene glycol (PEG), glycogen, 1,2-propanediol, dimethyl sulfoxide (DMSO), dimethylformamide (DMF), polyoxyethylene sorbitan monolaurate, such as polysorbate 20, polysorbate 40 and/or polysorbate 80, T4 gene 32 protein and dithiothreitol (DTT).

In an embodiment, the reverse transcription is conducted in the presence of a PEG having an average molecular weight selected within an interval of from 300 Da to 100,000 Da, preferably within an interval of from 1,000 to 25,000 Da, and more preferably within an interval of from 7,000 Da to 9,000 Da, such as 8000 Da. PEG, such as PEG 8000, acts a crowding agent causing a reduction in the effective reaction volume. This increases the enzymatic reaction rates. The addition of PEG may therefore increase the sensitivity of the method.

In some embodiments, the TSO comprises, from a 5′ end to a 3′ end, the amplification primer site, the identification tag, the UMI and the multiple predefined nucleotides. In some embodiments, the identification tag may serve as the amplification primer site (i.e., where the identification is employed as both an identification tag and an amplification primer site), such that the TSO includes a novel identification tag, UMI and the multiple predefine nucleotides. In such instances, the TSO does not include separate amplification primer site. As such, in some instances the TSO comprises a unique identification tag that can identify 5′ reads from complex mixtures, a UMI, and multiple predefined nucleotides, such as three rGs, wherein the unique identification tag also serves as a primer site for PCR amplification

In an embodiment, the amplification primer site of the TSO comprises a portion of a transposase motif sequence, such as a transposase 5 (Tn5) motif sequence. The Tn5 transposase cuts DNA molecules and adds the following sequences at either end of each DNA fragment:

(SEQ ID NO: 9)
5′-TCGTCGGCAGCGTCAGATGTGTATAAGAGACAG-3′
(SEQ ID NO: 10)
5′-GTCTCGTGGGCTCGGAGATGTGTATAAGAGACAG-3′

The portion of the Tn5 motif sequence thereby constitutes a portion of any of the above two sequences. For instance, the portion of the Tn5 motif sequence is preferably a 3′ portion of any of the above two sequences. Hence, in an embodiment, the portion of the Tn5 motif sequence comprises, preferably consists of, 5′-AGAGACAG-3′. This particular amplification primer site is compatible with ILLUMINA® Nextera P5 index primers.

In an embodiment, the identification tag of the TSO comprises a nucleotide sequence that does not exist in the transcriptome of a cell, or other RNA source, from which the RNA molecule originates. Hence, the identification tag is thereby unique and does not exist in the source material, e.g., transcriptome of the source cell, from which the RNA molecule was derived. This common identification tag can thereby be used to identify 5′ reads from a complex mixture of nucleic acid molecules.

In an embodiment, the identification tag comprises, preferably consists of, 5′-ATTGCGCAATG-3′ (SEQ ID NO: 11). This identification tag does not exist in the human transcriptome nor in the mouse transcriptome.

In an embodiment, the UMI of the TSO is a random n₁n₂n₃ . . . nₖ sequence, wherein nᵢ, i=1 . . . k, is one of adenine (A), thymidine (T), cytosine (C) and guanine (G). In an embodiment, k is from 4 up to 12, preferably from 6 up to 10, such as 8. With k=8, 65,5536 unique UMIs are possible using the nucleotides A, T, C and G. The UMI serves to reduce the quantitative bias introduced by amplification.

In an embodiment, the multiple predefined nucleotides of the TSO are three ribonucleotides, preferably three guanine ribonucleotides, i.e., rGrGrG. In alternative embodiments, the multiple predefined nucleotides are other ribonucleotides than guanine ribonucleotides, such as rC, rA or rU, e.g., rCrCrC, rArArA or rUrUrU in the case of three ribonucleotides. In further alternative embodiment, other guanine nucleotides than guanine ribonucleotides are used as the multiple predefined nucleotides as mentioned in the foregoing. For instance, at least one the multiple predefined nucleotides could be an LNA.

In a particular embodiment, the TSO thereby comprises, preferably consists of, the following sequence 5′-AGAGACAGATTGCGCAATGNNNNNNNNrGrGrG-3′ (SEQ ID NO:12).

In an embodiment, the cDNA synthesis primer is an oligo-dT primer, i.e., comprises multiple dTs. In a particular embodiment, the oligo-dT primer is an anchored oligo-dT primer.

The oligo-dT primer, preferably anchored oligo-dT primer, is complementary to and capable of hybridizing to a poly-A tail of the RNA molecule. In the case of an anchored oligo-dT primer, the oligo-dT primer comprises at least one additional selective nucleotide. As is well known in the art, an eukaryotic mRNA typically contains, from a 5′-end to a 3′-end, a cap, a 5′ untranslated region (UTR), the coding sequence (CDS), a 3′ UTR and the poly-A tail. This means that the anchored oligo-dT primer preferably comprises at least one nucleotide that is complementary to the last nucleotide(s) in the 3′ UTR or, in the case the mRNA molecule lacks a 3′ UTR, to the last nucleotide(s) in the CDR, in addition to the poly-A tail.

In an embodiment, instead of the being an oligo-dT primer, the cDNA synthesis primer is a gene specific primer, such that the oligo-dT domain described above is replaced by a gene specific sequence, i.e., a sequence that hybridizes to a known sequence in a gene of interest.

In an embodiment, the cDNA synthesis, e.g., oligo-dT, primer comprises, from a 5′ end to a 3′ end, a primer site, (T)ₚ, V, and N. V is selected from the group consisting of A, C and G, N is selected from the group consisting of A, C, G and T, and p is a positive number selected within an interval of from 10 to 50, preferably from 15 to 45, and more preferably from 20 to 40, such as 30.

In an embodiment, the primer site comprises a nucleotide sequence that does not exist in the transcriptome of a cell, or other source, from which the RNA molecule originates. In a particular embodiment, the primer site comprises, preferably consists of, 5′-ACGAGCATCAGCAGCATACGA-3′ (SEQ ID NO: 13). This primer site does not exist in the human transcriptome nor in the mouse transcriptome.

In a particular embodiment, the cDNA synthesis primer comprises, preferably consists of, the following sequence

(SEQ ID NO: 14)
5′-ACGAGCATCAGCAGCATACGA(T)ₚVN-3′.

The purpose of the VN of the anchored cDNA synthesis, e.g., oligo-dT, primer is to avoid random and multiple poly-T priming on poly-A tails. As a consequence, the anchored oligo-dT primer will bind to the 5′-end portion of poly-A tails since it includes at least one nucleotide that is complementary to the 3′-end of the 3′ UTR or the 3′-end of the CDS of the RNA molecule.

In an embodiment, step S1 of FIG. 8 comprises hybridizing, for each RNA molecule of a plurality of RNA molecules, the cDNA synthesis primer to the RNA molecule and synthesizing a respective cDNA strand complementary to at least a portion of the RNA molecule to form a respective RNA-cDNA intermediate. In this embodiment, step S2 comprises performing the template switching reaction by contacting the respective RNA-cDNA intermediate with a respective TSO under conditions suitable for extension of the respective cDNA strand using the respective TSO as template to form a respective extended cDNA strand complementary to the at least a portion of the RNA molecule and the respective TSO. In this embodiment, each TSO comprises the amplification primer site, the identification tag, a UMI, and the multiple predefined nucleotides. Each TSO comprises a UMI that is unique for the TSO and different from UMIs of other TSOs. In these embodiments, the total number of TSOs that have different UMIs may vary, where the collection of UMI varying TSOs ranges in some instances from 100 to 250,000, such as 1,000 to 100,000, including 10,000 to 75,000. The number of UMIs employed for a given sample may vary and may be selected with respect to the complexity of the sample. For example, fewer UMIs may be employed with less complex samples, while more UMIs may be employed with samples of greater complexity.

Thus, the present invention can be used to prepare cDNA molecules from a mixture of multiple different RNA molecules. In such a case, one and the same cDNA synthesis primer is preferably used whereas the TSOs used have different UMIs but preferably the same amplification primer site, the same common identification tag and the same multiple predefined nucleotides. For instance, a set of 65,536 unique TSOs with different UMIs can be obtained with a UMI length of 8 nucleotides.

In an embodiment, the method also comprises lysing (e.g., as described above) a cell to release RNA molecules as shown in FIG. 1A. The RNA molecules are preferably poly(A) containing RNA molecules, such as mRNA molecules, and are typically present in and released from the cytoplasm of the lysed cell. Any known cell lysing method can be used to release RNA molecules from the cell. The lysing method may involve usage of enzymes, detergents and/or chaotropic agent. Alternatively, or in addition, mechanical disruption of the cell membrane could be used, such as by repeated freezing and thawing and/or sonication. For instance, Triton X-100 could be used as detergent when lysing the cell.

FIG. 1A shows the reverse transcription and template switching reaction of steps S1 and S2 in FIG. 8. In an embodiment, the method also comprises amplifying the extended cDNA strand using a forward primer (also referred to as first forward primer or first forward amplification primer herein) and a reverse primer (also referred to as first reverse primer or first reverse amplification primer herein), which is schematically illustrated as PCR pre-amplification in FIG. 1A.

The amplification of the extended cDNA strand could be used serially with regard to steps S1 and S2, i.e., after formation of the extended cDNA strand. In another embodiment, the amplification of the extended cDNA strand is performed in the same reaction mix and/or simultaneous as the reverse transcription reaction and template switching reaction.

In an embodiment, the forward primer comprises the amplification primer site and the identification tag. In an embodiment, the forward primer comprises, from a 5′ end to a 3′ end, the Tn5 motif sequence and the identification tag. In a particular embodiment, the forward primer comprises, preferably consists of, 5′-TCGTCGGCAGCGTCAGATGTGTATAAGAGACAGATTGCGCAATG-3′ (SEQ ID NO: 15).

In an embodiment, the reverse primer comprises the primer site of the cDNA synthesis, e.g., oligo-dT, primer, or at least a portion thereof. Hence, in an embodiment, the reverse primer comprises, preferably consists of, 5′-ACGAGCATCAGCAGCATACGA-3′ (SEQ ID NO: 16).

The amplification step is preferably a PCR-based amplification using a polymerase, such as a Taq polymerase or a Phu polymerase or other DNA polymerases. Non-limiting, but illustrative, examples of polymerases that could be used in the PCR-based amplification include Phusion High Fidelity DNA polymerase, Platinum SuperFi DNA polymerase, Q5 High Fidelity DNA polymerase, KAPA HiFi HotStart DNA polymerase, and TERRA™ PCR Direct polymerase.

In an embodiment, the method also comprises, see FIG. 1B, fragmenting the resultant amplified cDNA molecules, e.g., using a fragmenting protocol as described above, followed by tagging the resultant fragments, e.g., for NGS. In some instances fragmenting and tagging the extended cDNA strand or an amplified version thereof is accomplished in a tagmentation process using a transposase and at least one tagging adapter to form tagged cDNA fragments.

In a particular embodiment, this fragmenting and tagging step comprises fragmenting and tagging the extended cDNA strand or the amplified version thereof in the tagmentation process using Tn5 and a first tagging adapter comprising a read 1 sequencing primer site and the amplification primer site and a second tagging adapter comprising a read 2 sequencing primer site and the amplification primer site. In a particular embodiment, the first tagging adapter comprises, preferably consists of, 5′-TCGTCGGCAGCGTCAGATGTGTATAAGAGACAG-3′ (SEQ ID NO: 17) and the second tagging adapter comprises, preferably consists of, 5′-GTCTCGTGGGCTCGGAGATGTGTATAAGAGACAG-3′ (SEQ ID NO: 18).

Transposase (EC 2.7.7) is an enzyme that binds to the end of a transposon and catalyzes the movement of the transposon to another part of the genome by a cut and paste mechanism or a replicative transposition mechanism. Tn5 is a transposase having simultaneous tagging and fragmentation properties. Accordingly, in addition to tagging cDNA molecules, such a transposase could further reduce the length of the cDNA molecules to achieve a length more suitable for the subsequent sequencing of the cDNA molecules. Other transposes than Tn5 could be used including, for instance, Mu transposase and Tn7 transposase.

The tagged cDNA fragments may then be amplified as shown in FIG. 1B in presence of a forward amplification primer (also referred to as second forward primer or second forward amplification primer herein) and a reverse amplification primer (also referred to as second reverse primer or second reverse amplification primer herein).

In an embodiment, the second forward amplification primer comprises, from a 5′ end to a 3′ end, a P5 sequence 5′-AATGATACGGCGACCACCGA-3′ (SEQ ID NO: 19), an i5 index and a portion of the read 1 sequencing primer site. In a particular embodiment, the i5 index is preferably selected from the group consisting of N501: TAGATCGC, N502: CTCTCTAT, N503: TATCCTCT, N504: AGAGTAGA, N505: GTAAGGAG, N506: ACTGCATA, N507: AAGGAGTA and N508: CTAAGCCT. Hence, the second forward amplification primer preferably comprises, or consists of, the following sequence 5′-AATGATACGGCGACCACCGANNNNNNNNTCGTCGGCAGCGTC-3′ (SEQ ID NO: 20), wherein NNNNNNNN represents the i5 index.

The second reverse amplification primer preferably comprises, from a 5′ end to a 3′ end, a P7 sequence 5′-CAAGCAGAAGACGGCATACGAGAT-3′ (SEQ ID NO: 21), an i7 index and a portion of the read 2 sequencing primer site. In a particular embodiment, the i7 index is preferably selected from the group consisting of N701: TAAGGCGA, N702: CGTACTAG, N703: AGGCAGAA, N704: TCCTGAGC, N705: GGACTCCT, N706: TAGGCATG, N707: CTCTCTAC, N708: CAGAGAGG, N709: GCTACGCT, N710: CGAGGCTG, N711: AAGAGGCA and N712: GTAGAGGA. Hence, the second reverse amplification primer preferably comprises, or consists of, the following sequence 5′-CAAGCAGAAGACGGCATACGAGATNNNNNNNNGTCTCGTGGGCTCGG-3′ (SEQ ID NO: 22), wherein NNNNNNNN represents the i7 index.

The amplified tagged cDNA fragments may then be sequenced as indicated in FIG. 1B by addition of at least one sequencing primer. The at least one sequencing primer preferably has a sequence corresponding to or complementary to at least a portion of the at least one tagging adapter.

In an embodiment, the at least one sequencing primer is selected among sequencing primers that can be used in ILLUMINA® sequencing technology, and in particular be used in ILLUMINA® sequencing technology of DNA sequences prepared with a Nextera DNA library prep kit. Examples of such sequencing primers include ILLUMINA® BP10—Read 1 primer, ILLUMINA® BP11—Read 2 primer and ILLUMINA® BP14—Index 1 primer and Index 2 primer.

In an embodiment, ILLUMINA® sequencing technology could be used to sequence at least a portion of the amplified tagged cDNA fragments by synthesis. Sequence By Synthesis (SBS) uses four fluorescently labeled nucleotides to sequence the amplified tagged cDNA fragments on a flow cell surface in parallel. During each sequencing cycle, a single labeled deoxynucleoside triphosphate (dNTP) is added to the nucleic acid chain. The nucleotide label serves as a terminator for polymerization so after each dNTP incorporation, the fluorescent dye is imaged to identify the base and then enzymatically cleaved to allow incorporation of the next nucleotide. More information of the ILLUMINA® sequencing technology can be found in Technology Spotlight: ILLUMINA® Sequencing [9].

Another aspect of the invention relates to a method for preparing a cDNA library. The method comprises preparing tagged cDNA fragments from RNA molecules, preferably of a single cell, as described in the foregoing and also shown in FIGS. 1A and 1B. This method also comprises tuning a percentage of the tagged cDNA fragments corresponding to a 5′ end portion of the extended cDNA strands.

Thus, the percentage of the tagged cDNA fragments that corresponds to the 5′ end portion of the extended cDNA strands and thereby comprise a respective UMI and the identification tag is tuned. In other words, the ratio between the number of tagged cDNA fragments that corresponds to the 5′ end portion of the extended cDNA strands and the total number of tagged cDNA fragments can be tuned or controlled.

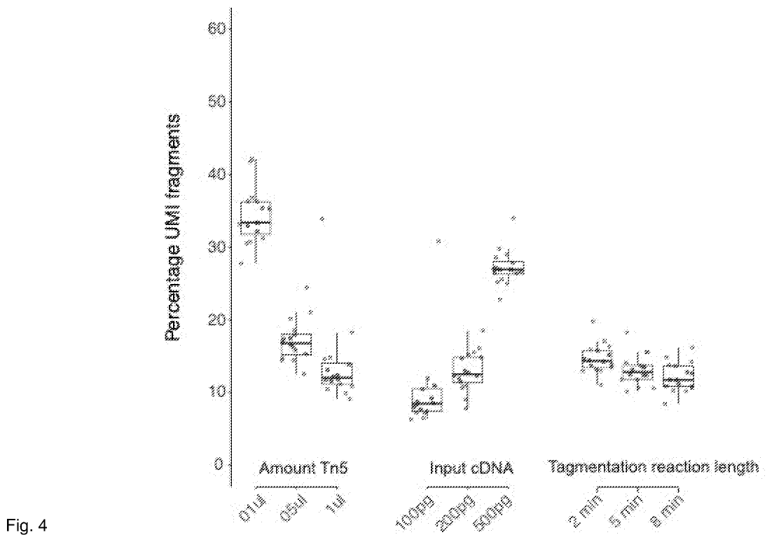

Experimental data as presented herein, see FIG. 4, show that the tuning can be performing by controlling or tuning the tagmentation efficiency, such as by controlling or selecting the amount of Tn5 transposase present in the fragmentation and tagging step, controlling or selecting the amount of input cDNA in the fragmentation and tagging step and/or controlling or selecting the reaction time of the in the fragmentation and tagging step. For instance, the Tn5-to-cDNA ratio could be controlled or selected to control or tune the tagmentation efficiency.

Different applications may make use of different extents of UMI vs. internal reads, therefore the ability to control the percentage of 5′ end reads is an advantageous feature. For example, applications that would make use of the high sensitivity of the invention to quantify gene expression would like to achieve as high as possible percentage of 5′ end fragments, whereas, for example, analyses of allelic transcription needs both internal reads for capturing genetic variation between alleles combined with UMI for gene quantification. Hence, the ability of being able control the percentage of 5′ end reads is an advantageous feature of the invention.

In an alternative embodiment, the balance between 5′ end fragments and internal fragments may be adjusted by amplifying the extended cDNA strand using a forward primer (also referred to as first forward primer or first forward amplification primer herein) and a reverse primer (also referred to as first reverse primer or first reverse amplification primer herein), wherein the forward primer comprises a biotin or other capture moiety. The resultant 5′ end fragments may then be separated from the internal fragments by capture of the biotin containing fragments on, for example, streptavidin beads. Libraries for sequencing may then be prepared separately using the methods described herein for the 5′ end fragments, captured on the beads and the internal fragments remaining unbound to the beads. The separate libraries may then be pooled in any appropriate ratio of interest to adjust the ratio of 5′end fragments to internal fragments.

A further aspect of the invention relates to methods for preparing nucleic acid fragments. In embodiments of such aspects, the methods include hybridizing a cDNA synthesis primer to a ribonucleic acid (RNA) molecule and synthesizing a cDNA strand complementary to at least a portion of the RNA molecule to form an RNA-cDNA intermediate, e.g., as described above; performing a template switching reaction by contacting the RNA-cDNA intermediate with a template switching oligonucleotide (TSO) under conditions suitable for extension of the cDNA strand using the TSO as template to form an extended cDNA strand complementary to the at least a portion of the RNA molecule and the TSO, wherein the TSO comprises an amplification primer site, an identification tag, a unique molecular identifier (UMI) and multiple predefined nucleotides, e.g., as described above; producing double-stranded cDNA from the extended cDNA strand, e.g., via PCR amplification, such as described above; and fragmenting the double-stranded cDNA, e.g., as described above, to produce nucleic acid fragments comprising a first population of 5′ UMI comprising fragments and a second population of internal fragments. Where fragmenting is accomplished via tagmentation, the resultant first population of 5′ UMI comprising fragments and a second population of internal fragments may include tagging adaptors that are added to the ends of the fragments during the tagmentation step. Where fragmenting is accomplished via other protocols, e.g., as described above, the methods may include tagging the first population of 5′ UMI comprising fragments and a second population of internal fragments with tagging adaptors, e.g., via ligation protocols, non ligation protocols, etc. The methods of these aspects may include simultaneously producing nucleic acid fragments from a plurality of distinct RNAs of a RNA sample, such as mRNAs of single cell.

In some embodiments, the resultant 5′ UMI comprising fragments and a second population of internal fragments may be sequenced, e.g., as described above. In such instances, the methods may include distinguishing sequencing reads of the first population of 5′ UMI comprising fragments from sequencing reads of the internal fragments by the presence of the identification tag sequence. In other words, reads obtained from fragments that include the identification tag sequence may be identified as arising from 5′ UMI comprising fragments, and reads obtained from fragments that lack the identification tag sequence may be identified as arising from internal fragments.

In some embodiments, the methods further comprise constructing the full-length sequence of the RNA from sequencing reads of both the 5′ UMI comprising and internal fragments. In such instances, the methods may include pairing a 5′ UMI containing read with a first read from a first internal fragment whose 5′ end aligns with the 3′ end of the 5′ UMI containing read. The resultant composite read may then be paired with a second read from a second internal fragment whose 5′ end aligns with the 3′ end of the read from the first internal fragment. The process may be continued until a complete read of the sequence of the RNA is obtained. Of course, the internal reads employed in such instances are sequencing reads of internal fragments produced from the same RNA from which the 5′UMI comprising fragments were produced.

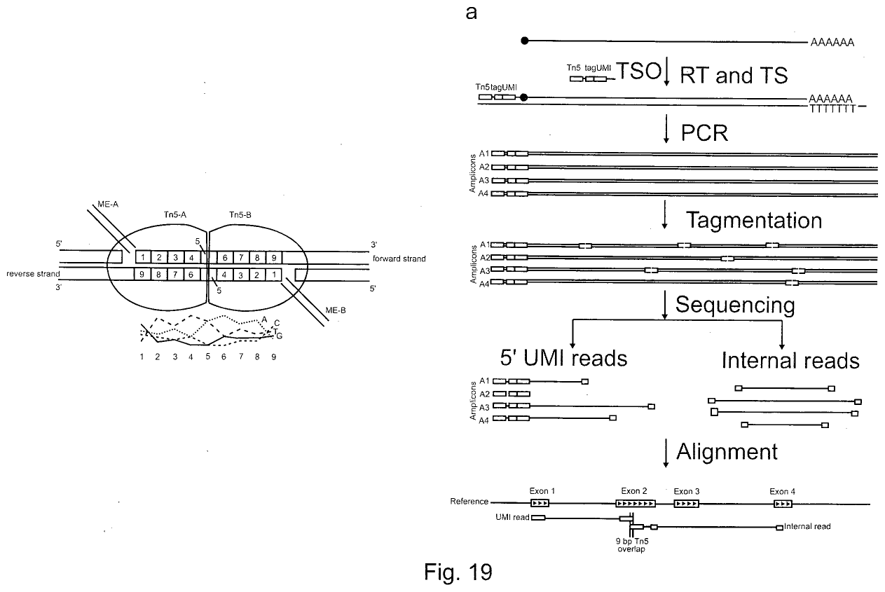

An embodiment of the above methods is illustrated in FIG. 19. As shown in FIG. 19, first strand cDNA is produced from an initial mRNA using a first strand primer and a TSO comprising a Tn5 motif comprising primer site, a unique tag, and UMI, and performing reverse transcription and template switching, e.g., as described above. Following PCR amplification, the resultant double stranded cDNAs are subjected to a tagmentation step to produce first population of 5′ UMI comprising fragments and a second population of internal fragments. The resultant fragments are then sequenced to obtain 5′ UMI reads and internal reads, all from the same RNA. The 5′UMI reads and internal reads are then aligned to construct the full sequence of the RNA. As shown in FIG. 19, not only are the 5′ fragments unique due to the UMI, such that they can be used to help build transcript models using combinations of paired end reads of these fragments, which will have different 3′ ends generated via tagmentation, but since the point of breakage of the original full length cDNA by the transposon is itself unique, the point of breakage can serve as an additional “UMI” to essentially allow linkage of a unique set of 5′ fragments to a unique set of internal reads. This feature can then be extended by analogy to the break on the 3′ side of this first internal fragment, so that one can add the next set of internal fragments 3′ of the first and so on to essentially walk all the way down the transcript from 5′ end to 3′ end. As shown in FIG. 19, when tagmentation is used to generate the fragments, the mechanism of tagmenation creates a staggered break in the DNA such that the 9 bases at the fragmentation point are repeated on the fragment pair coming from each side of the breakpoint. This 9-base signature may be employed in practicing methods of the invention to help identify pairs of adjacent fragments that were originally derived from the same molecule.

Following obtaining of the sequencing reads, e.g., as described above, the methods may further include one or more additional steps that employ the sequencing reads. For example, embodiments of the methods further include assigning an isoform to the RNA. As such, methods may include determining to which of several potential isoforms a given sequences belongs. Accordingly, methods may include distinguishing mRNAs that are produced from the same locus but are different in their transcription start sites (TSSs), protein coding DNA sequences (CDSs) and/or untranslated regions (UTRs).

In embodiments, the methods further include identifying at least a first single nucleotide polymorphism (SNP) of the RNA. In such instances, the methods may include identifying a second or more SNPs of the RNA. In such instances, the methods include setting a phase relationship of the first and second SNPs. For example, using methods of the invention one can determine with certainty that two SNPs seen in the same linked reads are from the same original molecule. As such, the SNPs must by definition be on the same chromosome. Accordingly, one can set their phase relationship to each other. This ability may be employed in evaluating inherited genetic disorders, e.g., cancer or other inherited genetic disorders, where one might want to know if a particular gene has been mutated on both maternal and paternal chromosomes (i.e. generating a null homozygous mutation), or only on one (heterozygous mutant/wild-type). Such methods may be employed in clinical applications, e.g., diagnosis and/or therapy.

In embodiments, the methods include identifying the RNA as the product of a gene fusion, i.e., the product of a hybrid gene formed from two previously separate genes, such as may be formed as a result of translocation, interstitial deletion, or chromosomal inversion.

Embodiments of the methods may include normalizing the populations of fragments. Normalization may be viewed as the process of equalizing the DNA library concentration for multiplexing and addresses the problems of library over-representation or under-representation in a given multiplexed composition. In a given multiplex NGS workflow, normalization may be employed at different stages, including normalization of the concentration of input DNA/RNA, size distribution of library fragments as well as the normalization of library preparation concentration prior to pooling. In some instances, a normalization protocol as described in PCT Application Serial No. PCT/US2019/064477 filed on Dec. 4, 2019, the disclosure of which is herein incorporated by reference, is employed.

A further aspect of the invention relates to a kit for preparing cDNA. The kit comprises a cDNA synthesis primer configured to hybridize to an RNA molecule to enable synthesis of a cDNA strand complementary to at least a portion of the RNA molecule to form an RNA-cDNA intermediate. The kit also comprises a TSO comprising an amplification primer site, an identification tag, a UMI and multiple predefined nucleotides.

In an embodiment, the TSO is configured to act as a template in a template switching reaction comprising extension of the cDNA strand to form an extended cDNA strand complementary to the at least a portion of the RNA molecule and the TSO.

In an embodiment the kit includes a set of TSOs that differ from each other by UMI, e.g., as described above.

In an embodiment, the kit also comprises a reverse transcriptase. The reverse transcriptase is preferably selected among the previously described examples of reverse transcriptases.

In an embodiment, the kit comprises ribonucleotides, preferably guanine ribonucleotides, at a concentration selected within an interval of from 0.05 mM to 10 mM, preferably within an interval of from 0.1 mM to 3 mM.

In an embodiment, the kit comprises a mixture dATP, dGTP, dTTP and dCTP. The mixture preferably comprises a same concentration of dATP, dGTP and dTTP and a concentration of dCTP that is X mM higher than the same concentration of dATP, dGTP and dTTP. In an embodiment, X is selected within an interval of from 0.05 mM to 10 mM, preferably within an interval of from 0.1 mM to 3 mM.

In an embodiment, the kit comprises a magnesium salt in a concentration selected within an interval of from 0.1 mM to 20 mM, preferably within an interval of from 1 mM to 10 mM, and more preferably within an interval of from 2 mM to 5 mM. The magnesium salt is preferably selected among the previously described examples of magnesium salts.

In an embodiment, the kit comprises a chloride salt selected from the group consisting of NaCl, CsCl, and a mixture thereof. In an embodiment, the kit does not comprise any KCl.

In an embodiment, the kit comprises at least one reverse transcription and/or amplification enhancer. The at least one such enhancer is preferably selected among the previously described examples of enhancers. In an embodiment, the kit comprises a PEG having an average molecular weight selected within an interval of from 300 Da to 100,000 Da, preferably within an interval of from 1,000 to 25,000 Da, and more preferably within an interval of from 7,000 Da to 9,000 Da, such as 8000 Da.

In an embodiment, the kit comprises a forward primer and a reverse primer for amplifying the extended cDNA strand.

In an embodiment, the kit comprises a transposase and at least one tagging adapter for fragmenting and tagging the extended cDNA strand or an amplified version thereof in a tagmentation process to form tagged cDNA fragments.

In an embodiment, the kit comprises a forward amplification primer and a reverse amplification primer for amplifying the tagged cDNA fragments.

In an embodiment, the kit comprises at least one sequencing primer, preferably having a sequence corresponding to or complementary to at least a portion of the at least one tagging adapter for sequencing the amplified tagged cDNA fragments.

The kit can advantageously be used in the method for preparing cDNA according to the invention.

In addition to the above-mentioned components, a subject kit may further include instructions for using the components of the kit, e.g., to practice the subject methods as described above. In addition, the kit may further include programming for analysis of results including, e.g., counting unique molecular species, etc. The instructions and/or analysis programming may be recorded on a suitable recording medium. The instructions and/or programming may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXAMPLES

### I. Example 1

A. Materials and Methods

Cell Cultures

HEK293FT cells (Invitrogen) were cultured in complete Dulbecco's modification of Eagle medium (DMEM) medium containing glucose and glutamine (Gibco), supplemented with 10% fetal bovine serum (FBS), 0.1 mM MEM Non-essential Amino Acids (Gibco), 1 mM sodium pyruvate (Gibco) and 100 μg/mL pencillin/streptomycin (Gibco). Cells were passaged using TrypLE express (Gibco).

Single Cell Isolation and Lysis

Single cell suspensions were prepared by dissociating HEK293FT cells using TrypLE Express resuspended in phosphate-buffered saline (PBS) and stained with propidium Iodide (PI), to distinguish live and dead cells. Single cells were sorted into 96 or 384-well plates using a BD FACSMelody 100 μm nozzle (BD Bioscience), containing 3 μL lysis buffer. The lysis buffer consisted of 1 U/μL recombinant RNase inhibitor (RRI) (Takara), 0.15% Triton X-100 (Sigma), 0.5 mM dNTP/each (Thermo Scientific), 1 μM Smartseq3 OligodT primer (5′-Biotin-ACGAGCATCAGCAGCATACGAT₃₀VN-3′ (SEQ ID NO: 11); IDT), and 0.05 μL of 1:40.000 diluted External RNA Controls Consortium (ERCC) spike-in mix 1 (Ambion). Immediately after sorting the plates were spun down before storage at −80° C.

Generation of Smart-seq2 libraries Smart-seq2 cDNA libraries were generated according the published protocol [10-11]. Tagmentation was performed with similar cDNA input and volumes as for Smartseq3 described below.

Reverse Transcription

To facilitate lysing and denaturation of the RNA, the plates of cells were incubated at 72° C. for 10 min, and immediately placed on ice afterwards. Next, 5 μL of reverse transcription mix, containing 50 mM Tris-HCl pH 8.3 (Sigma), 75 mM NaCl (Ambion) or CsCl (Sigma), 1 mM GTP (Thermo Scientific), 3 mM MgCl₂ (Ambion), 10 mM DTT (Thermo Scientific), 5% PEG (Sigma), 1 U/μL RRI (Takara), 2 μM Smartseq3 template switching oligo (TSO) (5′-Biotin-AGAGACAGATTGCGCAATGNNNNNNNNrGrGrG-3′ (SEQ ID NO: 23); IDT) and 2 U/μL Maxima H-minus reverse transcriptase enzyme (Thermo Scientific), were added to each sample. In other variants of the protocol without PEG, the reverse transcription mix also contained 1 mM dCTP (Thermo Scientific). Reverse transcription and template switching were carried out at 42° C. for 90 min followed by 10 cycles of 50° C. for 2 min and 42° C. for 2 min. The reaction was terminated by incubating at 85° C. for 5 min.

PCR Pre-Amplification

PCR pre-amplification was performed directly after reverse transcription by adding 17 μL of PCR mix consisting of 2×KAPA HiFI HotStart Readymix (0.5 U DNA polymerase, 0.3 mM dNTPs, 2.5 mM MgCl₂ at 1× in 25 μL reaction) (Roche), 0.1 μM Smartseq3 forward PCR primer (5′-TCGTCGGCAGCGTCAGATGTGTATAAGAGACAGATTGCGCAATG-3′ (SEQ ID NO: 24); IDT), 0.1 μM Smartseq3 reverse PCR primer (5′-ACGAGCATCAGCAGCATACGA-3′ (SEQ ID NO: 25); IDT). PCR was cycled as following; 3 min at 98° C. for initial denaturation, 20 cycles of 20 secs at 98° C., 30 sec at 65° C., 6 min at 72° C. Final elongation was performed for 5 min at 72° C.

Library Preparation and Sequencing

Following PCR pre-amplification all samples were purified with AMpure XP beads (Beckman Coulter) at a 1:0.8 sample to bead ratio. The final elution was performed in 15 μL H₂O (Thermo Scientific). Library size distributions were checked on a High sensitivity DNA chip (Agilent Bioanalyzer), while cDNA was quantified using the Quant-iT PicoGreen dsDNA Assay Kit (Thermo Scientific). 200 pg of pre-amplified cDNA was used for tagmentation carried out with Nextera XT DNA Sample preparation kit (Illumina) at ⅕ volume according to manufacturer's protocol. After tagmentation, the samples were pooled, and the pool purified with Ampure XP beads at 1:0.6 ratio. All libraries were sequenced at 1×76 bp single-end on a high output flow cell using the ILLUMINA® NextSeq500 instrument.

Read Alignments and Gene-Expression Estimation Raw non-demultiplexed fastq files were processed using zUMIs 2.0 with STAR, to generate expression profiles for both the 5′ ends containing UMIs as well as full length non-UMI data. To extract the UMI specific reads in zUMIs find_pattern: ATTGCGCAATG (SEQ ID NO: 26) was specified for file1 as well as base_definition: cDNA(23-75) and UMI(12-19) in the YAML file. UMIs were counted using a Hamming distance of 1 to collapse UMIs. To retrieve full length profiles in zUMis the base_definiton in the YAML file was set to cDNA(1-75) for file1. Experiments containing HEK293FT cells were aligned and mapped to the human genome (hg38) with gene annotations from ENSEMBL GRCh38.91.

Reagents and Conditions tested for Smartseq3

| | |
|---|---|
| Lysis conditions | Concentration |
| TX-100 | 0.1%, 0.15%, 0.2% |
| Guanidine-HCI | 100 mM, 250 mM, |
| | 300 mM, 350 mM, |
| | 400 mM, 450 mM, |
| | 500 mM, 750 mM, |
| | 1 M, 1.25M, 1.5 M, 2 M |
| Bovine Serum Albumin (BSA) | 0.01 mg/ml, 0.025 mg/ml, |
| | 0.05 mg/ml, 0.1 mg/ml, |
| | 0.25 mg/ml, 0.5 mg/ml, |
| | 1.0 mg/ml, 2.0 mg/ml |
| RNAse Inhibitor | 0.5 μ/pL, 1.0 μ/pL, |
| | 1.3 μ/pL |
| PEG8000K (percent according to | 2%, 2.5%, 4%, 5%, 6%, |
| | 7.5%, 9%, 10% |
| Lysis+RTvol) | |
| Oligo dT (Table 1) | 0.1 μM, 0.2 μM, 0.25 μM, 0.4 μM, 0.5 μM, 0.75 μM, |
| | 1 μM, 1.25 μM, 2 μM, |
| | 4 μM |
| Proteinase K | 0.01-1.25 pg/pL |
| dNTPs (mM/each) | 0.05 mM, 0.1 mM, |
| | 0.25 mM, 0.3 mM, |
| | 0.4 mM, 0.5 mM, |
| | 0.75 mM, 0.8 mM, |
| | 1 mM, 1.25 mM, 1.5 mM, 1.75 mM, 2 mM |
| Lysis Temperature | |
| 37° C. for 30 min | |
| 72° C. for 1 min | |
| 72° C. for 3 min | |
| 72° C. for 10 min | |
| 72° C. for 20 min | |
| 50° C. for 10 min, 80° C. for 10 min | |
| RT buffers | Concentration |
| Tris-HCI pH 7.0 | 50 mM |
| Tris-HCI pH 7.5 | 50 mM |
| Tris-HCI pH 8.0 | 20 mM, 25 mM, 30 mM, |
| | 35 mM, 40 mM, |
| | 50 mM, 65 mM, |
| Tris-HCI pH 8.3 | 20 mM, 25 mM, 30 mM, |
| | 35 mM, 40 mM, |
| | 50 mM, 65 mM, |
| Tris-Acetate pH 7.5 | 50 mM |
| TAPS-NaOH pH 8.4 | 50 mM |
| TAPS-KOH pH 8.4 | 50 mM |
| Alkaline chlorides and salts | Concentration |
| KCI | 75 mM |
| NaCI | 25 mM, 50 mM, 75 mM, |
| | 100 mM, 125 mM, |
| | 150 mM |
| CsCI | 75 mM |
| LiCI | 75 mM |
| Ammonium sulfate | 10 mM, 20 mM, 30 mM |
| Mg/Mn sources | Concentration |
| MgCl₂ | 2 mM, 2.5 mM, 3 mM, |
| | 3.5 mM, 4 mM, 4.5 mM, |
| | 5 mM, 6 mM, 9 mM, |
| | 10 mM, 12 mM |
| MgOAc | 2 mM, 2.5 mM, 3 mM, |
| | 3.5 mM, 4 mM, 4.5 mM, |
| | 5 mM, 6 mM, 9 mM |
| MgSO₂ | 2 mM, 2.5 mM, 3 mM, |
| | 3.5 mM, 4 mM, 4.5 mM, |
| | 5 mM, 6 mM, 9 mM |
| MnCl₂ | 0.1 mM, 0.25 mM, 0.5 mM, 0.75 mM, |
| | 1 mM, 2 mM, 3 mM, 6 mM |
| dNTPINTP additives in RT | Concentration |
| GTP | 0-4 mM |
| dGTP | 0-4 mM |
| GMP | 0-4 mM |
| dGMP | 0-4 mM |
| dCTP | 0-4 mM |
| CTP | 0-4 mM |
| CMP | 0-4 mM |
| dCMP | 0-4 mM |
| RTIPCR enhancers | Concentration |
| Betaine | 0.35 M, 0.5 M, 1 M, 1.2 M, 1.3 M, |
| | 1.5 M, 2 M |
| Bovine Serum Albumin (BSA) | 0.01 mg/ml, 0.025 mg/ml, |
| | 0.05 mg/ml, 0.1 mg/ml, |
| | 0.25 mg/ml, 0.5 mg/ml |
| Glycerol | 2%, 5%, 7%, 10% |
| PEG300 | 1-10% |
| PEG400 | 1-10% |
| PEG8000 | 1-10% |
| Glycogen | 5% |
| 1,2 Propanediol | 5% |
| DMSO | 1-5% |
| DMF | 1-10% |
| Tween-20 | 0.01-0.5% |
| T4 Gene 32 Protein | 0.01-1 pg/pL |
| Dithiothreitol (DTT) | 5 mM, 7.5 mM, 10 mM, |
| | 12.5 mM, 15 mM |
| Reverse Transcriptases | Concentration |
| Superscriptll | 2-10 μ/pL |
| SuperscriptIll | 10 μ/pL |
| SuperscriptIV | 10 μ/pL |
| RevertAid H-minus | 2-10 μ/pL |
| Protoscript II | 10 μ/pL |
| Maxima H-minus | 2-10 μ/pL |
| EpiScript | 10 μ/pL |
| RNAse Inhibitor | Concentration |
| Recombinant RNAse Inhibitor (RRI) | 0.5 μ/pL, 1 μ/pL |
| RNAseOUT | 0.5 μ/pL, 1 U/L |
| TSO (Table 2) | Concentration |
| | 0.5 μM, 0.75 μM, 1 μM, |
| | 1.5 μM, 2 μM, 4 μM, |
| | 8 μM, 12 μM, 16 μM |
| RT temperatures | |
| 42° C. for 90 min, 10x(50° C. for 2 min, | |
| 42° C. for 2 min), 70° C. for 15 min | |
| 50° C. for 90 min, 10x(50° C. for 2 min, | |
| 42° C. for 2 min), 85° C. for 5 min | |
| 48° C. for 90 min, 10x(50° C. for 2 min, | |
| 42° C. for 2 min), 85° C. for 5 min | |
| 45° C. for 90 min, 10x(50° C. for 2 min, | |
| 42° C. for 2 min), 85° C. for 5 min | |
| 42° C. for 90 min, 10x(50° C. for 2 min, | |
| 42° C. for 2 min), 85° C. for 5 min | |
| 42° C. for 90 min, 10x(50° C. for 2 min, | |
| 42° C. for 2 min) | |
| 42° C. for 60 min, 10x(50° C. for 2 min, | |
| 42° C. for 2 min), 85° C. for 5 min | |
| 42° C. for 45 min, 10x(50° C. for 2 min, | |
| 42° C. for 2 min), 85° C. for 5 min | |
| 42° C. for 30 min, 10x(50° C. for 2 min, | |
| 42° C. for 2 min), 85° C. for 5 min | |
| 42° C. for 15 min, 10x(50° C. for 2 min, | |
| 42° C. for 2 min), 85° C. for 5 min | |
| 50° C. for 30 min, 10x(35° C. for 2 min, | |
| 55° C. for 2 min), 85° C. for 5 min | |
| 10x(50° C. for 2 min, 42° C. for 2 min), | |
| 85° C. for 5 min | |
| 10x(50° C. for 3 min, 42° C. for 2 min), | |
| 85° C. for 5 min | |
| 10x(50° C. for 2 min, 42° C. for 4 min), | |
| 85° C. for 5 min | |
| 10x(42° C. for 3 min, 55° C. for 2 min, | |
| 37° C. for 1 min), 85° C. for 5 min | |
| 25° C. for 90 min, 10x(50° C. for 2 min, | |
| 25° C. for 2 min), 85° C. for 5 min | |
| 42° C. for 90 min, 85° C. for 5 min | |
| 45° C. for 90 min, 85° C. for 5 min | |
| 48° C. for 90 min, 85° C. for 5 min | |
| 50° C. for 60 min, 85° C. for 5 min | |
| 50° C. for 90 min, 85° C. for 5 min | |
| 53° C. for 90 min, 85° C. for 5 min | |
| 55° C. for 90 min, 85° C. for 5 min | |
| 10x(42° C. for 10 min, 15° C. for 2 min), | |
| 10x(50° C. for 2 min, 42° C. for 2 min), | |
| 85° C. for 5 min | |
| 10x(42° C. for 7 min, 15° C. for 2 min), | |
| 10x(50° C. for 2 min, 42° C. for 2 min), | |
| 85° C. for 5 min | |
| 10x(55° C. for 7 min, 15° C. for 2 min), | |
| 10x(50° C. for 2 min, 42° C. for 2 min), | |
| 85° C. for 5 min | |
| 10x(50° C. for 3 min, 65° C. for 3 min, | |
| 45° C. for 3 min, 42° C. for 3 min), 85° C. | |
| for 5 min | |
| 10x(50° C. for 3 min, 45° C. for 3 min, | |
| 42° C. for 3 min, 37° C. for 3 min), | |
| 85° C. for 5 min | |
| 10x(42° C. for 10 min, 37° C. for 2 min), | |
| 10x(50° C. for 2 min, 42° C. for 2 min), | |
| 85° C. for 5 min | |
| 50° C. for 10 min, 3x(8° C. for 15 sec, | |
| 15° C. for 45 sec, 20° C. for 45 sec, 30° C. | |
| for 30 sec, 42° C. | |
| for 2 min, 50° C. for 3 min), 50° C. for 5 | |
| min, 85° C. for 5 min | |
| RT-PCR temperatures | |
| 42° C. for 90 min, 10x(50° C. for 2 min, | |
| 42° C. for 2 min), 98° C. for 3 min | |
| 20x(98° C. for 20 sec, 63° C. for 30 sec, | |
| 72° C. for 6 min), 72° C. for 5 min | |
| 45° C. for 90 min, 10x(50° C. for 2 min, | |
| 42° C. for 2 min), 98° C. for 3 min | |
| 20x(98° C. for 20 sec, 63° C. for 30 sec, | |
| 72° C. for 6 min), 72° C. for 5 min | |
| 42° C. for 90 min, 10x(50° C. for 2 min, | |
| 42° C. for 2 min), 98° C. for 3 min | |
| 20x(98° C. for 20 sec, 65° C. for 30 sec, | |
| 72° C. for 6 min), 72° C. for 5 min | |
| 45° C. for 90 min, 10x(50° C. for 2 min, | |
| 42° C. for 2 min), 98° C. for 3 min | |
| 20x(98° C.for 20 sec, 65° C. for 30 sec, | |
| 72° C. for 6 min), 72° C. for 5 min | |
| 42° C. for 90 min, 10x(50° C. for 2 min, | |
| 42° C. for 2 min), 98° C. for 3 min | |
| 20x(98° C. for 20 sec, 67° C. for 30 sec, | |
| 72° C. for 6 min), 72° C. for 5 min | |
| 45° C. for 90 min, 10x(50° C. for 2 min, | |
| 42° C. for 2 min), 98° C. for 3 min | |
| 20x(98° C. for 20 sec, 67° C. for 30 sec, | |
| 72° C. for 6 min), 72° C. for 5 min | |
| PCR Kits and polymerases | Concentration |
| KAPA HiFi HotStart PCR Kit | |
| Terra PCR Direct Polymerase Kit | |
| KAPA HiFi PCR Kit | |
| Q5 High Fidelity DNA polymerase | |
| Platinum SuperFi DNA polymerase | |
| Phusion High Fidelity DNA polymerase | |
| PCR Primers (Table 3) | 0.05 μM, 0.08 μM, 0.1 μM |
| PCR temperatures | |
| 98° C. for 3 min 20x(98° C. for 20 sec, | |
| 65° C. for 30 sec, 72° C. for 6 min), | |
| 72° C. for 5 min | |
| 98° C. for 3 min 18x(98° C. for 20 sec, | |
| 65° C. for 30 sec, 72° C. for 6 min), | |
| 72° C. for 5 min | |
| 98° C. for 3 min 20x(98° C. for 20 sec, | |
| 60° C. for 30 sec, 72° C. for 6 min), | |
| 72° C. for 5 min | |
| 98° C. for 3 min 20x(98° C. for 20 sec, | |
| 61° C. for 30 sec, 72° C. for 6 min), | |
| 72° C. for 5 min | |
| 98° C. for 3 min 20x(98° C. for 20 sec, | |
| 62° C. for 30 sec, 72° C. for 6 min), | |
| 72° C. for 5 min | |
| 98° C. for 3 min 20x(98° C. for 20 sec, | |
| 63° C. for 30 sec, 72° C. for 6 min), | |
| 72° C. for 5 min | |
| 98° C. for 3 min 20x(98° C. for 20 sec, | |
| 64° C. for 30 sec, 72° C. for 6 min), | |
| 72° C. for 5 min | |
| 98° C. for 3 min 20x(98° C. for 20 sec, | |
| 65° C. for 30 sec, 72° C. for 6 min), | |
| 72° C. for 5 min | |
| 98° C. for 3 min 20x(98° C. for 20 sec, | |
| 66° C. for 30 sec, 72° C. for 6 min), | |
| 72° C. for 5 min | |
| 98° C. for 3 min 20x(98° C. for 20 sec, | |
| 67° C. for 30 sec, 72° C. for 6 min), | |
| 72° C. for 5 min | |
| 98° C. for 3 min 20x(98° C. for 20 sec, | |
| 68° C. for 30 sec, 72° C. for 6 min), | |
| 72° C. for 5 min | |
| 98° C. for 3 min 20x(98° C. for 20 sec, | |
| 69° C. for 30 sec, 72° C. for 6 min), | |
| 72° C. for 5 min | |
| 98° C. for 3 min 20x(98° C. for 20 sec, | |
| 70° C. for 30 sec, 72° C. for 6 min), | |
| 72° C. for 5 min | |
| 98° C. for 3 min 20x(98° C. for 20 sec, | |
| 71° C. for 30 sec, 72° C. for 6 min), | |
| 72° C. for 5 min | |
| 98° C. for 3 min 20x(98° C. for 20 sec, | |
| 72° C. for 30 sec, 72° C. for 6 min), | |
| 72° C. for 5 min | |
| 98° C. for 3 min 20x(98° C. for 20 sec, | |
| 60° C. for 15 sec, 72° C. for 6 min), | |
| 72° C. for 5 min | |
| 98° C. for 3 min 20x(98° C. for 20 sec, | |
| 61° C. for 15 sec, 72° C. for 6 min), | |
| 72° C. for 5 min | |
| 98° C. for 3 min 20x(98° C. for 20 sec, | |
| 62° C. for 15 sec, 72° C. for 6 min), | |
| 72° C. for 5 min | |
| 98° C. for 3 min 20x(98° C. for 20 sec, | |
| 63° C. for 15 sec, 72° C. for 6 min), | |
| 72° C. for 5 min | |
| 98° C. for 3 min 20x(98° C. for 20 sec, | |
| 64° C. for 15 sec, 72° C. for 6 min), | |
| 72° C. for 5 min | |
| 98° C. for 3 min 20x(98° C. for 20 sec, | |
| 65° C. for 15 sec, 72° C. for 6 min), | |
| 72° C. for 5 min | |
| 98° C. for 3 min 20x(98° C. for 20 sec, | |
| 66° C. for 15 sec, 72° C. for 6 min), | |
| 72° C. for 5 min | |
| 98° C. for 3 min 20x(98° C. for 20 sec, | |
| 67° C. for 15 sec, 72° C. for 6 min), | |
| 72° C. for 5 min | |
| 98° C. for 3 min 20x(98° C. for 20 sec, | |
| 68° C. for 15 sec, 72° C. for 6 min), | |
| 72° C. for 5 min | |
| 98° C. for 3 min 20x(98° C. for 20 sec, | |
| 69° C. for 15 sec, 72° C. for 6 min), | |
| 72° C. for 5 min | |
| for 3 min 20x(98° C. for 20 sec, 70° C. for | |
| 15 sec, 72° C. for 6 min), 72° C. for 5 min | |
| for 3 min 20x(98° C. for 20 sec, 71° C. for | |
| 15 sec, 72° C. for 6 min), 72° C. for 5 min | |
| for 3 min 20x(98° C. for 20 sec, 72° C. for | |
| 15 sec, 72° C. for 6 min), 72° C. for 5 min | |

TABLE 1
oligo dT oligonucleotides

| Name | Description | Sequence |
|---|---|---|
| Biold412_ | NoHsHit2.dT | 5′-Biotin-idSp-idSp-idSp-ACGAGCATCAGCAGCATACGATTTTTTTTTTTTTTTTTTTTTTTTTTTTTTVN- |
| Sp | 30 | 3′ (SEQ ID NO: 27) |
| 412 | NoHsHit2.dT | 5′-Biotin-ACGAGCATCAGCAGCATACGATTTTTTTTTTTTTTTTTTTTTTTTTTTTTTVN-3′ (SEQ ID |
| | 30 | NO: 28) |
| 412_ | NoHsHit2.dT | 5′-Biotin-TEG-ACGAGCATCAGCAGCATACGATTTTTTTTTTTTTTTTTTTTTTTTTTTTTTVN-3′ (SEQ ID |
| BioTEG | 30 | NO: 29) |
| 412_no_ | NoHsHit2.dT | 5′-ACGAGCATCAGCAGCATACGATTTTTTTTTTTTTTTTTTTTTTTTTTTTTTVN-3′ (SEQ ID NO: 30) |
| Mod | 30 | |
| 412_no_ | NoHsHit2.dT | 5′-Biotin-ACGAGCATCAGCAGCATACGATTTTTTTTTTTTTTTTTTTTTTTTTTTTTTV-3′ (SEQ ID |
| N | 30_noN | NO: 31) |
| 412_no_ | NoHsHit2.dT | 5′-Biotin-ACGAGCATCAGCAGCATACGATTTTTTTTTTTTTTTTTTTTTTTTTTTTTT-3′ (SEQ ID |
| VN | 30_noVN | NO: 32) |
| M25 | NoHsHit2.dT | 5′-Biotin-ACGAGCATCAGCAGCATACGATTTTTTTTTTTTTTTTTTTTVN-3′ |
| | 20 | (SEQ ID NO: 33) |
| M29 | NoHsHit2.dT | 5′-Biotin-ACGAGCATCAGCAGCATACGATTTTTTTTTTTTTTTTTTTTTTTTTTTTTTTTTTTTTTTTVN-3′ |
| | 40 | (SEQ ID NO: 34) |
| M30 | NoHsHit2.dT | 5′-Biotin- |
| | 48 | ACGAGCATCAGCAGCATACGATTTTTTTTTTTTTTTTTTTTTTTTTTTTTTTTTTTTTTTTTTTTTTTTVN- |
| | | 3′ (SEQ ID NO: 35) |

idSp: Internal 1′,2′-dideoxyribose (dSpacer)

TABLE 2
TSO

| Name | Description | Sequence |
|---|---|---|
| 407 | NoHsHit. | 5′-Biotin-idSp-idSp-idSp-ACTGGAAGAGTGCCATCAGArGrGG-3′ (SEQ ID NO: 36) |
| | rGrGG | |
| 439 | NexPap_H6_ | 5′-Biotin-AGAGACAGATTGCGCAATGHHHHHHrG+GG-3′ (SEQ ID NO: 37) |
| | rG+GG | |
| 442 | NexPap_bio_ | 5′-Biotin-TEG-AGAGACAGATTGCGCAATGHHHHHHrG+GG-3′(SEQ ID NO: 38) |
| | H6_JG+GG | |
| 443 | NexPap_bio_ | 5′-Biotin-TEG-AGAGACAGATTGCGCAATGHHHHHHrGrGrG-3′ (SEQ ID NO: 39) |
| | H6_rGrGrG | |
| 444 | NexPap_bio_ | 5′-Biotin-TEG-AGAGACAGATTGCGCAATGHHHHHHHrGrGrG-3′ (SEQ ID NO: 40) |
| | H7_rGrGrG | |
| 445 | NexPap_bio_ | 5′-Biotin-TEG-AGAGACAGATTGCGCAATGHHHHHHHrG+GG-3′ |
| | H7_JG+GG | (SEQ ID NO: 41) |
| 450 | NexPap_bio_ | 5′-Biotin-TEG-AGAGACAGATTGCGCAATGrGrGrG-3′ |
| | rGrGrG | (SEQ ID NO: 42) |
| 451 | NexPap_bio_ | 5′-Biotin-TEG-AGAGACAGATTGCGCAATGNNNNrGrGrG-3′ |
| | N4_rGrGrG | (SEQ ID NO: 43) |
| 452 | NexPap_bio_ | 5′-Biotin-TEG-AGAGACAGATTGCGCAATGHHHHHHHHrGrGrG-3′ |
| | H8_rGrGrG | (SEQ ID NO: 44) |
| 453 | NexPap_bio_ | 5′-Biotin-TEG-AGAGACAGATTGCGCAATGHHHHHHHHHrGrGrG-3′ |
| | H9_rGrGrG | (SEQ ID NO: 45) |
| 454 | NexPap_bio_ | 5′-Biotin-TEG-AGAGACAGATTGCGCAATGHHHHHHHHHHrGrGrG-3′ |
| | H10_rGrGrG | (SEQ ID NO: 46) |
| 455 | NexPap_bio_ | 5′-Biotin-TEG-AGAGACAGATTGCGCAATGNNNNHHHHrGrGrG-3′ |
| | N4H4_rGrGrG | (SEQ ID NO: 47) |
| M1 | SS3_TSO_N6 | 5′-Biotin-AGAGACAGATTGCGCAATGNNNNNNrGrGrG-3′ |
| | | (SEQ ID NO: 48) |
| M2 | SS3_TSO_N6_ | 5′-Biotin-rArGrArGrArCrArGrArUrUrGrCrGrCrArArUrGrNrNrNrNrNrNrGrGrG-3′ |
| | RNA | (SEQ ID NO: 49) |
| M3 | SS3_TSO_H6_ | 5′-Biotin-rArGrArGrArCrArGrArUrUrGrCrGrCrArArUrGrHrHrHrHrHrHrGrGrG-3′ |
| | RNA | (SEQ ID NO: 50) |
| M7 | SS3_TSO_N7 | 5′-Biotin-AGAGACAGATTGCGCAATGNNNNNNNrGrGrG-3′ (SEQ ID NO: 51) |
| M8 | SS3_TSO_N8 | 5′-Biotin-AGAGACAGATTGCGCAATGNNNNNNNNrGrGrG-3′ (SEQ ID NO: 52) |
| M9 | SS3_TSO_N8_ | 5′-AGAGACAGATTGCGCAATGNNNNNNNNrGrGrG-3′ (SEQ ID NO: 53) |
| | noMod | |
| M10 | SS3_TSO_N8_ | 5′-Biotin-AGAGACAGATTGCGCAATGNNNNNNNNrNrGrG-3′ |
| | rNrGrG | (SEQ ID NO: 54) |
| M11 | SS3_N8_tag_ | 5′-Biotin-ATTGCGCAATGNNNNNNNNrGrGrG-3′ |
| | noNex | (SEQ ID NO: 55) |
| M12 | SS3_N8_tag_ | 5′-Biotin-AGATTGCGCAATGNNNNNNNNrGrGrG-3′ |
| | 24bp | (SEQ ID NO: 56) |
| M13 | SS3_N8_tag_ | 5′-Biotin-ACAGATTGCGCAATGNNNNNNNNrGrGrG-3′ |
| | 26 bp | (SEQ ID NO: 57) |
| M14 | SS3_N8_tag_ | 5′-Biotin-AGACAGATTGCGCAATGNNNNNNNNrGrGrG-3′ |
| | 28 bp | (SEQ ID NO: 58) |
| M15 | SS3_N8_tag_ | 5′-Biotin-TAAGAGACAGATTGCGCAATGNNNNNNNNrGrGrG-3′ (SEQ ID NO: 59) |
| | 32 bp | |
| M16 | SS3_N8_tag_ | 5′-Biotin-TATAAGAGACAGATTGCGCAATGNNNNNNNNrGrGrG-3′(SEQ ID NO: 60) |
| | 34 bp | |
| M17 | SS3_N8_tag_ | 5′-Biotin-TGTATAAGAGACAGATTGCGCAATGNNNNNNNNrGrGrG-3′ |
| | 36 bp | (SEQ ID NO: 61) |
| M19 | SS3_N8_AT_ | 5′-Biotin-AGAGACAGATTGCGCAATGNNNNNNNNATrGrGrG-3′ (SEQ ID NO: 62) |
| | rGrGrG | |
| M20 | SS3_N8_AT_ | 5′-Biotin-ACAGATTGCGCAATGNNNNNNNNATrGrGrG-3′ |
| | 28 bp_rGrGrG | (SEQ ID NO: 63) |
| M21 | SS3_TSO_N8 | 5′-Biotin-AGAGACAGATTGCGCAATGNNNNNNNNrGrGrG-3′ |
| | | (SEQ ID NO: 64) |
| 439 m | SS3_TSO_H6_ | 5′-Biotin-AGAGACAGATTGCGCAATGHHHHHHrGrG+G-3′ |
| | LNA | (SEQ ID NO: 65) |
| M22 | SS3_TSO_N8_ | 5′-Biotin-AGAGACAGATTGCGCAATGNNNNNNNNrGrG+G-3′ |
| | LNA | (SEQ ID NO: 66) |
| M23 | SS3_i5NitIndx4_ | 5′-Biotin-AGAGACAGATTGCGCAATG/i5NiTInd/i5NiTInd/i5NiTInd/i5NiTInd/ |
| | rGrGrG | rGrGrG-3′ (SEQ ID NO: 67) |
| M24 | SS3_I5NitIndx4_ | 5′-Biotin-AGAGACAGATTGCGCAATG/i5NiTInd/i5NiTInd/i5NiTInd/i5NiTInd/ |
| | N4_rGrGrG | NNNNrGrGrG-3′ (SEQ ID NO: 68) |
| M31 | SS3_TSO_N8_ | 5′-Biotin-AGAGACAGATTGCGCAATGNNNNNNNNrGrGG-3′ (SEQ ID NO: 69) |
| | rGrGG | |

idSp: Internal 1′,2′-dideoxyribose (dSpacer)
i5NiTInd: Internal 5-Nitroindole Universal base
rN: ribonucleotide
+N: LNA

TABLE 3
PCR oligonucleotides

| Name | Description | Sequence |
|---|---|---|
| 414 | NoHsHit2.Rv | 5′-ACGAGCATCAGCAGCATACGA-3′ (SEQ ID NO: 70) |
| 441 | NexPap_Fw6 | 5′-TCGTCGGCAGCGTCAGATGTGTATAAGAGACAGATTGCGCAATG-3′ (SEQ ID NO: 71) |
| M26 | NoHsHit2.Rv_ | 5′-TCGTATGCACGAGCATCAGCAGCATACGA-3′ (SEQ ID NO: 72) |
| | Hairpin | |
| M27 | NexPap_Fw6_ | 5′-GCGCAATCTCGTCGGCAGCGTCAGATGTGTATAAGAGACAGATTGCGCAATG-3′ (SEQ ID |
| | Hairpin1 | NO: 73) |
| M28 | NexPap_Fw6_ | 5′-CATTGCGCAATTCGTCGGCAGCGTCAGATGTGTATAAGAGACAGATTGCGCAATG-3′ (SEQ ID |
| | Hairpin2 | NO: 74) |
| M32 | NexPap_Thiol_ | 5′-ThioMC6-D-TCGTCGGCAGCGTCAGATGTGTATAAGAGACAGATTGCGCAATG-3′ (SEQ ID |
| | Fw6 | NO: 75) |
| M33 | NoHsHit2. | 5′-ThioMC6-D-ACGAGCATCAGCAGCATACGA-3′ (SEQ ID NO: 76) |
| | Thiol.Rv | |

ThioMC6-D: 5′ Thiol Modifier C6 S-S

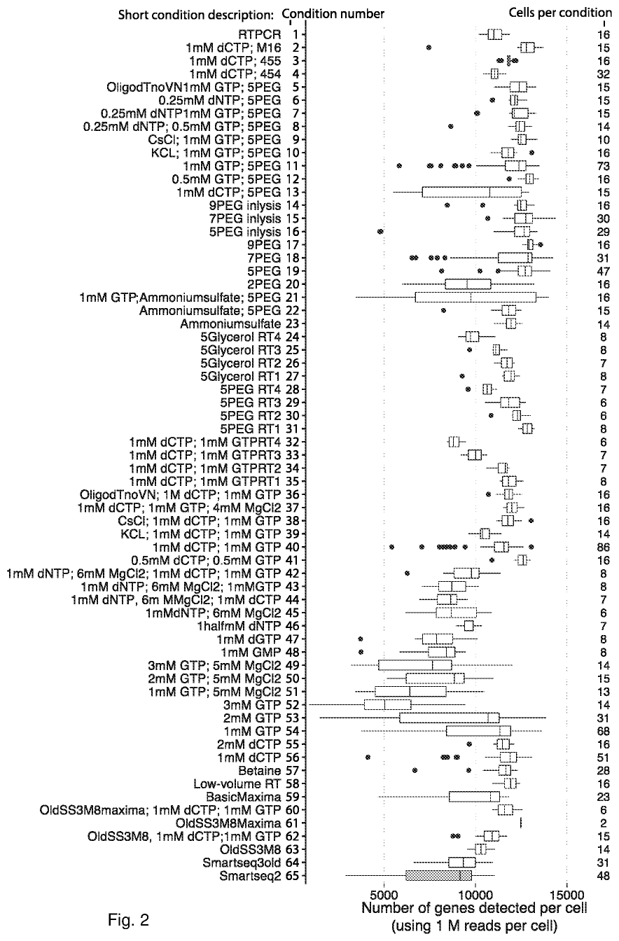

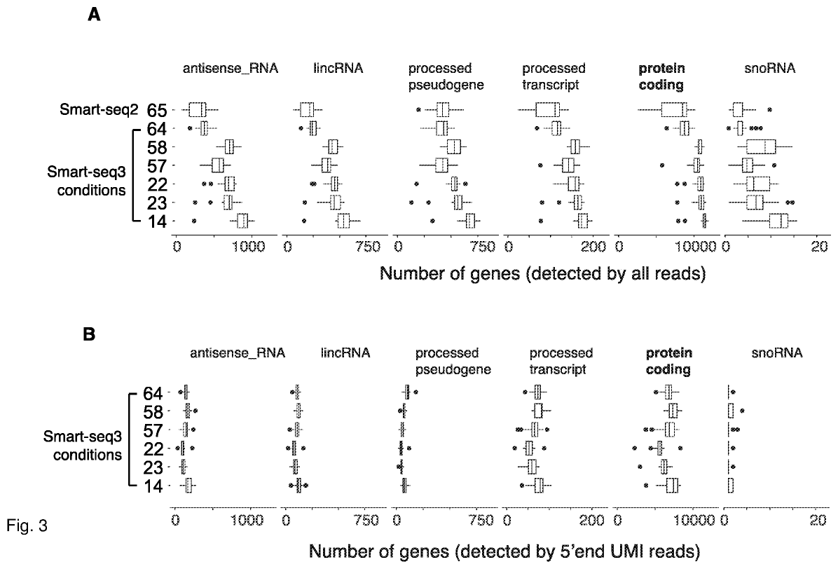

TABLE 4
experimental reaction conditions used for FIGS. 2 and 3

| | | | ERCC (μL of | | | | |
|---|---|---|---|---|---|---|---|
| Condition | Name | # cells | 40.000X) | OligodT | | | |
| 1 | RTPCR (0.4X KAPA buffer) | 16 | 0.1 | Smartseq3 OligodT30VN | | | |
| 2 | 1 mMdCTPM16 | 16 | 0.1 | Smartseq3 OligodT30VN | | | |
| 3 | 1 mMdCTP455 | 16 | 0.1 | Smartseq3 OligodT30VN | | | |
| 4 | 1 mMdCTP454 | 16 | 0.1 | Smartseq3 OligodT30VN | | | |
| 5 | OligodTnoVN1 mMGTP5PEG | 16 | 0.1 | Smartseq3 OligodT30_noVN | | | |
| 6 | 0.25 mMdNTP5% PEG | 16 | 0.1 | Smartseq3 OligodT30VN | | | |
| 7 | 0.25 mMdNTP1 mMGTP5% PEG | 16 | 0.1 | Smartseq3 OligodT30VN | | | |
| 8 | 0.25 mMdNTP0.5 mMGTP5% PEG | 16 | 0.1 | Smartseq3 OligodT30VN | | | |
| 9 | CsCL1 mMGTP5% PEG | 16 | 0.05 | Smartseq3 OligodT30VN | | | |
| 10 | KCL1 mMGTP5% PEG | 16 | 0.05 | Smartseq3 OligodT30VN | | | |
| 11 | 1 mMGTP5PEG | 80 | 0.1 | Smartseq3 OligodT30VN | | | |
| 12 | 0.5 mMGTP5% PEG | 16 | 0.1 | Smartseq3 OligodT30VN | | | |
| 13 | 1 mMdCTP5PEG | 16 | 0.1 | Smartseq3 OligodT30VN | | | |
| 14 | 9% PEGinLysis | 16 | 0.1 | Smartseq3 OligodT30VN | | | |
| 15 | 7% PEGinLysis | 32 | 0.1 | Smartseq3 OligodT30VN | | | |
| 16 | 5% PEGinLysis | 31 | 0.1 | Smartseq3 OligodT30VN | | | |
| 17 | 9% PEG | 16 | 0.1 | Smartseq3 OligodT30VN | | | |
| 18 | 7% PEG | 32 | 0.1 | Smartseq3 OligodT30VN | | | |
| 19 | 5% PEG | 48 | 0.1 | Smartseq3 OligodT30VN | | | |
| 20 | 2% PEG | 16 | 0.1 | Smartseq3 OligodT30VN | | | |
| 21 | 1 mMGTPAmmoniumSulfate5PEG | 16 | 0.1 | Smartseq3 OligodT30VN | | | |
| 22 | AmmoniumSulfate5PEG | 16 | 0.1 | Smartseq3 OligodT30VN | | | |
| 23 | AmmoniumSulfate | 16 | 0.1 | Smartseq3 OligodT30VN | | | |
| 24 | 5% GlycerolRT4 | 8 | 0.1 | Smartseq3 OligodT30VN | | | |
| 25 | 5% GlycerolRT3 | 8 | 0.1 | Smartseq3 OligodT30VN | | | |
| 26 | 5% GlycerolRT2 | 8 | 0.1 | Smartseq3 OligodT30VN | | | |
| 27 | 5% GlycerolRT1 | 8 | 0.1 | Smartseq3 OligodT30VN | | | |
| 28 | 5% PEGRT4 | 8 | 0.1 | Smartseq3 OligodT30VN | | | |
| 29 | 5% PEGRT3 | 8 | 0.1 | Smartseq3 OligodT30VN | | | |
| 30 | 5% PEGRT2 | 8 | 0.1 | Smartseq3 OligodT30VN | | | |
| 31 | 5% PEGRT1 | 8 | 0.1 | Smartseq3 OligodT30VN | | | |
| 32 | 1 mMdCTP1 mMGTPRT4 | 8 | 0.1 | Smartseq3 OligodT30VN | | | |
| 33 | 1 mMdCTP1 mMGTPRT3 | 8 | 0.1 | Smartseq3 OligodT30VN | | | |
| 34 | 1 mMdCTP1 mMGTPRT2 | 8 | 0.1 | Smartseq3 OligodT30VN | | | |
| 35 | 1 mMdCTP1 mMGTPRT1 | 8 | 0.1 | Smartseq3 OligodT30VN | | | |
| 36 | OligodTnoVN1 mdCTP1 mMGTP | 16 | 0.1 | Smartseq3 OligodT30_noVN | | | |
| 37 | 1 mMdCTP+1 mMGTP4 | 16 | 0.1 | Smartseq3 OligodT30VN | | | |
| | mMMgCl2 | | | | | | |
| 38 | CsCL1 mMdCTP1 mMGTP | 16 | 0.05 | Smartseq3 OligodT30VN | | | |
| 39 | KCL1 mMdCTP1 mMGTP | 16 | 0.05 | Smartseq3 OligodT30VN | | | |
| 40 | 1 mMdCTP1 mMGTP | 88 | 0.1 | Smartseq3 OligodT30VN | | | |
| 41 | 0.5 mMdCTP0.5 mMGTP | 16 | 0.1 | Smartseq3 OligodT30VN | | | |
| 42 | 1 mMdNTP 6 mMMgCl2 1 | 8 | 0.1 | Smartseq3 OligodT30VN | | | |
| | mMdCTP 1 mMGTP | | | | | | |
| 43 | 1 mMdNTP 6 mMMgCl2 | 8 | 0.1 | Smartseq3 OligodT30VN | | | |
| | 1 mMGTP | | | | | | |
| 44 | 1 mMdNTP 6 mMMgCl2 | 8 | 0.1 | Smartseq3 OligodT30VN | | | |
| | 1 mMdCTP | | | | | | |
| 45 | 1 mMdNTP 6 mMMgCl2 | 8 | 0.1 | Smartseq3 OligodT30VN | | | |
| 46 | 1halfmMdNTP | 8 | 0.1 | Smartseq3 OligodT30VN | | | |
| 47 | 1 mMdGTP | 8 | 0.1 | Smartseq3 OligodT30VN | | | |
| 48 | 1 mMGMP | 8 | 0.1 | Smartseq3 OligodT30VN | | | |
| 49 | 3 mMGTP5 mMMgCl2 | 16 | 0.1 | Smartseq3 OligodT30VN | | | |
| 50 | 2 mMGTP5 mMMgCl2 | 16 | 0.1 | Smartseq3 OligodT30VN | | | |
| 51 | 1 mMGTP5 mMMgCl2 | 16 | 0.1 | Smartseq3 OligodT30VN | | | |
| 52 | 3 mMGTP | 16 | 0.1 | Smartseq3 OligodT30VN | | | |
| 53 | 2 mMGTP | 32 | 0.1 | Smartseq3 OligodT30VN | | | |
| 54 | 1 mMGTP | 72 | 0.1 | Smartseq3 OligodT30VN | | | |
| 55 | 2 mMdCTP | 16 | 0.05 | Smartseq3 OligodT30VN | | | |
| 56 | 1 mMdCTP | 56 | 0.1 | Smartseq3 OligodT30VN | | | |
| 57 | Betaine | 32 | 0.1 | Smartseq3 OligodT30VN | | | |
| 58 | LowVolRT | 16 | 0.1 | Smartseq3 OligodT30VN | | | |
| 59 | BasicMaxima | 24 | 0.1 | Smartseq3 OligodT30VN | | | |
| 60 | OldSS3 m8 maximal | 16 | 0.1 | Smartseq3 OligodT30VN | | | |
| | mMdCTp1 mMGTP | | | | | | |
| 61 | OldSS3 m8 maxima | 16 | 0.1 | Smartseq3 OligodT30VN | | | |
| 62 | OldSS3 m81 mMdCTp1 mMGTP | 16 | 0.1 | Smartseq3 OligodT30VN | | | |
| 63 | OldSS3 m8 | 16 | 0.1 | Smartseq3 OligodT30VN | | | |
| 64 | OldSmartseq3 | 32 | 0.1 | Smartseq3 OligodT30VN | | | |
| 65 | Smartseq2 | 48 | 0.1 | Smartseq2 OligodT30VN | | | |
| | Oligo-dT | dNTPs/each | | | | TSO amount | |
| Condition | amount (μM) | (mM) | Salt | TSO | UMI | (μM) | |
| 1 | 1 | 0.8 | NaCl | M8 (rGrGrG) | 8N | 2 | |
| 2 | 1 | 0.5 | NaCl | M16 (rGrGrG) | 8N | 2 | |
| 3 | 1 | 0.5 | NaCl | 455 (rGrGrG) | 4N4H | 2 | |
| 4 | 1 | 0.5 | NaCl | 454 (rGrGrG) | 10H | 2 | |
| 5 | 1 | 0.5 | NaCl | M8 (rGrGrG) | 8N | 2 | |
| 6 | 1 | 0.25 | NaCl | M8 (rGrGrG) | 8N | 2 | |
| 7 | 1 | 0.25 | NaCl | M8 (rGrGrG) | 8N | 2 | |
| 8 | 1 | 0.25 | NaCl | M8 (rGrGrG) | 8N | 2 | |
| 9 | 1 | 0.5 | CSCl | M8 (rGrGrG) | 8N | 2 | |
| 10 | 1 | 0.5 | CSCl | M8 (rGrGrG) | 8N | 2 | |
| 11 | 1 | 0.5 | NaCl | M8 (rGrGrG) | 8N | 2 | |
| 12 | 1 | 0.5 | NaCl | M8 (rGrGrG) | 8N | 2 | |
| 13 | 1 | 0.5 | NaCl | M8 (rGrGrG) | 8N | 2 | |
| 14 | 1 | 0.5 | NaCl | M8 (rGrGrG) | 8N | 2 | |
| 15 | 1 | 0.5 | NaCl | M8 (rGrGrG) | 8N | 2 | |
| 16 | 1 | 0.5 | NaCl | M8 (rGrGrG) | 8N | 2 | |
| 17 | 1 | 0.5 | NaCl | M8 (rGrGrG) | 8N | 2 | |
| 18 | 1 | 0.5 | NaCl | M8 (rGrGrG) | 8N | 2 | |
| 19 | 1 | 0.5 | NaCl | M8 (rGrGrG) | 8N | 2 | |
| 20 | 1 | 0.5 | NaCl | M8 (rGrGrG) | 8N | 2 | |
| 21 | 1 | 0.5 | NaCl/(NH₄)₂SO₄ | M8 (rGrGrG) | 8N | 2 | |
| 22 | 1 | 0.5 | NaCl/(NH₄)₂SO₄ | M8 (rGrGrG) | 8N | 2 | |
| 23 | 1 | 0.5 | NaCl/(NH₄)₂SO₄ | M8 (rGrGrG) | 8N | 2 | |
| 24 | 1 | 0.5 | NaCl | M8 (rGrGrG) | 8N | 2 | |
| 25 | 1 | 0.5 | NaCl | M8 (rGrGrG) | 8N | 2 | |
| 26 | 1 | 0.5 | NaCl | M8 (rGrGrG) | 8N | 2 | |
| 27 | 1 | 0.5 | NaCl | M8 (rGrGrG) | 8N | 2 | |
| 28 | 1 | 0.5 | NaCl | M8 (rGrGrG) | 8N | 2 | |
| 29 | 1 | 0.5 | NaCl | M8 (rGrGrG) | 8N | 2 | |
| 30 | 1 | 0.5 | NaCl | M8 (rGrGrG) | 8N | 2 | |
| 31 | 1 | 0.5 | NaCl | M8 (rGrGrG) | 8N | 2 | |
| 32 | 1 | 0.5 | NaCl | M8 (rGrGrG) | 8N | 2 | |
| 33 | 1 | 0.5 | NaCl | M8 (rGrGrG) | 8N | 2 | |
| 34 | 1 | 0.5 | NaCl | M8 (rGrGrG) | 8N | 2 | |
| 35 | 1 | 0.5 | NaCl | M8 (rGrGrG) | 8N | 2 | |
| 36 | 1 | 0.5 | NaCl | M8 (rGrGrG) | 8N | 2 | |
| 37 | 1 | 0.5 | NaCl | M8 (rGrGrG) | 8N | 2 | |
| 38 | 1 | 0.5 | KCl | M8 (rGrGrG) | 8N | 2 | |
| 39 | 1 | 0.5 | KCl | M8 (rGrGrG) | 8N | 2 | |
| 40 | 1 | 0.5 | NaCl | M8 (rGrGrG) | 8N | 2 | |
| 41 | 1 | 0.5 | NaCl | M8 (rGrGrG) | 8N | 2 | |
| 42 | 1 | 1 | NaCl | M8 (rGrGrG) | 8N | 2 | |
| 43 | 1 | 1 | NaCl | M8 (rGrGrG) | 8N | 2 | |
| 44 | 1 | 1 | NaCl | M8 (rGrGrG) | 8N | 2 | |
| 45 | 1 | 1 | NaCl | M8 (rGrGrG) | 8N | 2 | |
| 46 | 1 | 1.5 | NaCl | M8 (rGrGrG) | 8N | 2 | |
| 47 | 1 | 0.5 | NaCl | M8 (rGrGrG) | 8N | 2 | |
| 48 | 1 | 0.5 | NaCl | M8 (rGrGrG) | 8N | 2 | |
| 49 | 1 | 0.5 | NaCl | M8 (rGrGrG) | 8N | 2 | |
| 50 | 1 | 0.5 | NaCl | M8 (rGrGrG) | 8N | 2 | |
| 51 | 1 | 0.5 | NaCl | M8 (rGrGrG) | 8N | 2 | |
| 52 | 1 | 0.5 | NaCl | M8 (rGrGrG) | 8N | 2 | |
| 53 | 1 | 0.5 | NaCl | M8 (rGrGrG) | 8N | 2 | |
| 54 | 1 | 0.5 | NaCl | M8 (rGrGrG) | 8N | 2 | |
| 55 | 1 | 0.5 | NaCl | M8 (rGrGrG) | 8N | 2 | |
| 56 | 1 | 0.5 | NaCl | M8 (rGrGrG) | 8N | 2 | |
| 57 | 1 | 0.5 | NaCl | M8 (rGrGrG) | 8N | 2 | |
| 58 | 1 | 0.5 | NaCl | M8 (rGrGrG) | 8N | 2 | |
| 59 | 1 | 0.5 | NaCl | M8 (rGrGrG) | 8N | 2 | |
| 60 | 1 | 1.25 | NaCl | M8 (rGrGrG) | 8N | 2 | |
| 61 | 1 | 1.25 | NaCl | M8 (rGrGrG) | 8N | 2 | |
| 62 | 1 | 1.25 | NaCl | M8 (rGrGrG) | 8N | 2 | |
| 63 | 1 | 1.25 | NaCl | M8 (rGrGrG) | 8N | 2 | |
| 64 | 1 | 1.25 | NaCl | 439 (rG + GG) | 6H | 1 | |
| 65 | 1 | 1 | KCl | Smartseq2 LNA | — | 1 | |
| | | MgCl2 (total | PEG (8000K) | Other additives | RT vol | | |
| Condition | RT enzyme | reaction, mM) | % | in RT | (μL) | RT protocol | |
| 1 | Maxima H | 3 | — | 1 mM dCTP, 1 | 10 | 90′@ 42C., 10X | |
| | minus | | | mM GTP | | (2′@50C., 2′@42C.) | |
| 2 | Maxima H | 3 | — | 1 mM dCTP | 10 | 90′@ 42C., 10X | |
| | minus | | | | | (2′@50C., 2′@42C.) | |
| | | | | | | 5′@ 85C. | |
| 3 | Maxima H | 3 | — | 1 mM dCTP | 10 | 90′@ 42C., 10X | |
| | minus | | | | | (2′@50C., 2′@42C.) | |
| | | | | | | 5′@ 85C. | |
| 4 | Maxima H | 3 | — | 1 mM dCTP | 10 | 90′@ 42C., 10X | |
| | minus | | | | | (2′@50C., 2′@42C.) | |
| | | | | | | 5′@ 85C. | |
| 5 | Maxima H | 3 | 5 | 1 mM GTP | 10 | 90′@ 42C., 10X | |
| | minus | | | | | (2′@50C., 2′@42C.) | |
| | | | | | | 5′@ 85C. | |
| 6 | Maxima H | 3 | 5 | | 10 | 90′@ 42C., 10X | |
| | minus | | | | | (2′@50C., 2′@42C.) | |
| | | | | | | 5′@ 85C. | |
| 7 | Maxima H | 3 | 5 | 1 mM GTP | 10 | 90′@ 42C., 10X | |
| | minus | | | | | (2′@50C., 2′@42C.) | |
| | | | | | | 5′@ 85C. | |
| 8 | Maxima H | 3 | 5 | 0.5 mM GTP | 10 | 90′@ 42C., 10X | |
| | minus | | | | | (2′@50C., 2′@42C.) | |
| | | | | | | 5′@ 85C. | |
| 9 | Maxima H | 3 | 5 | 1 mM GTP | 10 | 90′@ 42C., 10X | |
| | minus | | | | | (2′@50C., 2′@42C.) | |
| | | | | | | 5′@ 85C. | |
| 10 | Maxima H | 3 | 5 | 1 mM GTP | 10 | 90′@ 42C., 10X | |
| | minus | | | | | (2′@50C., 2′@42C.) | |
| | | | | | | 5′@ 85C. | |
| 11 | Maxima H | 3 | 5 | 1 mM GTP | 10 | 90′@ 42C., 10X | |
| | minus | | | | | (2′@50C., 2′@42C.) | |
| | | | | | | 5′@ 85C. | |
| 12 | Maxima H | 3 | 5 | 0.5 mM GTP | 10 | 90′@ 42C., 10X | |
| | minus | | | | | (2′@50C., 2′@42C.) | |
| | | | | | | 5′@ 85C. | |
| 13 | Maxima H | 3 | 5 | 1 mM dCTP | 10 | 90′@ 42C., 10X | |
| | minus | | | | | (2′@50C., 2′@42C.) | |
| | | | | | | 5′@ 85C. | |
| 14 | Maxima H | 3 | 9 | | 10 | 90′@ 42C., 10X | |
| | minus | | | | | (2′@50C., 2′@42C.) | |
| | | | | | | 5′@ 85C. | |
| 15 | Maxima H | 3 | 7.5 | | 10 | 90′@ 42C., 10X | |
| | minus | | | | | (2′@50C., 2′@42C.) | |
| | | | | | | 5′@ 85C. | |
| 16 | Maxima H | 3 | 5 | | 10 | 90′@ 42C., 10X | |
| | minus | | | | | (2′@50C., 2′@42C.) | |
| | | | | | | 5′@ 85C. | |
| 17 | Maxima H | 3 | 9 | | 10 | 90′@ 42C., 10X | |
| | minus | | | | | (2′@50C., 2′@42C.) | |
| | | | | | | 5′@ 85C. | |
| 18 | Maxima H | 3 | 7.5 | | 10 | 90′@ 42C., 10X | |
| | minus | | | | | (2′@50C., 2′@42C.) | |
| | | | | | | 5′@ 85C. | |
| 19 | Maxima H | 3 | 5 | | 10 | 90′@ 42C., 10X | |
| | minus | | | | | (2′@50C., 2′@42C.) | |
| | | | | | | 5′@ 85C. | |
| 20 | Maxima H | 3 | 2.5 | | 10 | 90′@ 42C., 10X | |
| | minus | | | | | (2′@50C., 2′@42C.) | |
| | | | | | | 5′@ 85C. | |
| 21 | Maxima H | 3 | 5 | 1 mM GTP | 10 | 90′@ 42C., 10X | |
| | minus | | | | | (2′@50C., 2′@42C.) | |
| | | | | | | 5′@ 85C. | |
| 22 | Maxima H | 3 | 5 | — | 10 | 90′@ 42C., 10X | |
| | minus | | | | | (2′@50C., 2′@42C.) | |
| | | | | | | 5′@ 85C. | |
| 23 | Maxima H | 3 | — | — | 10 | 90′@ 42C., 10X | |
| | minus | | | | | (2′@50C., 2′@42C.) | |
| | | | | | | 5′@ 85C. | |
| 24 | Maxima H | 3 | — | 5% Glycerol | 10 | 90′@ 50C., 5′@ 85C. | |
| | minus | | | | | | |
| 25 | Maxima H | 3 | — | 5% Glycerol | 10 | 90′@ 48C., 5′@ 85C. | |
| | minus | | | | | | |
| 26 | Maxima H | 3 | — | 5% Glycerol | 10 | 90′@ 45C., 10X | |
| | minus | | | | | (2′@55C., 2′@45C.), | |
| | | | | | | 5′@ 85C. | |
| 27 | Maxima H | 3 | — | 5% Glycerol | 10 | 90′@ 42C., 10X | |
| | minus | | | | | (2′@50C., 2′@42C.), | |
| | | | | | | 5′@ 85C. | |
| 28 | Maxima H | 3 | 5 | | 10 | 90′@ 50C., 5′@ 85C. | |
| | minus | | | | | | |
| 29 | Maxima H | 3 | 5 | | 10 | 90′@ 48C., 5′@ 85C. | |
| | minus | | | | | | |
| 30 | Maxima H | 3 | 5 | | 10 | 90′@ 45C., 10X | |
| | minus | | | | | (2′@55C., 2′@45C.), | |
| | | | | | | 5′@ 85C. | |
| 31 | Maxima H | 3 | 5 | | 10 | 90′@ 42C., 10X | |
| | minus | | | | | (2′@50C., 2′@42C.), | |
| | | | | | | 5′@ 85C. | |
| 32 | Maxima H | 3 | — | 1 mM dCTP, 1 | 10 | 90′@ 50C., 5′@ 85C. | |
| | minus | | | mM GTP | | | |
| 33 | Maxima H | 3 | — | 1 mM dCTP, 1 | 10 | 90′@ 48C., 5′@ 85C. | |
| | minus | | | mM GTP | | | |
| 34 | Maxima H | 3 | — | 1 mM dCTP, 1 | 10 | 90′@ 45C., 10X | |
| | minus | | | mM GTP | | (2′@55C., 2′@45C.), | |
| | | | | | | 5′@85C. | |
| 35 | Maxima H | 3 | — | 1 mM dCTP, 1 | 10 | 90′@ 42C., 10X | |
| | minus | | | mM GTP | | (2′@50C., 2′@42C.), | |
| | | | | | | 5′@85C. | |
| 36 | Maxima H | 3 | — | 1 mM dCTP, 1 | 10 | 90′@ 42C., 10X | |
| | minus | | | mM GTP | | (2′@50C., 2′@42C.), | |
| | | | | | | 5′@ 85C. | |
| 37 | Maxima H | 4 | — | 1 mM dCTP, 1 | 10 | 90′@ 42C., 10X | |
| | minus | | | mM GTP | | (2′@50C., 2′@42C.), | |
| | | | | | | 5′@ 85C. | |
| 38 | Maxima H | 3 | — | 1 mM dCTP, 1 | 10 | 90′@ 42C., 10X | |
| | minus | | | mM GTP | | (2′@50C., 2′@42C.), | |
| | | | | | | 5′@ 85C. | |
| 39 | Maxima H | 3 | — | 1 mM dCTP, 1 | 10 | 90′@ 42C., 10X | |
| | minus | | | mM GTP | | (2′@50C., 2′@42C.), | |
| | | | | | | 5′@ 85C. | |
| 40 | Maxima H | 3 | — | 1 mM dCTP, 1 | 10 | 90′@ 42C., 10X | |
| | minus | | | mM GTP | | (2′@50C., 2′@42C.), | |
| | | | | | | 5′@ 85C. | |
| 41 | Maxima H | 3 | — | 0.5 mM dCTP, | 10 | 90′@ 42C., 10X | |
| | minus | | | 0.5 mM GTP | | (2′@50C., 2′@42C.), | |
| | | | | | | 5′@ 85C. | |
| 42 | Maxima H | 6 | — | 1 mM dCTP, 1 | 10 | 90′@ 42C., 10X | |
| | minus | | | mM GTP | | (2′@50C., 2′@42C.), | |
| | | | | | | 5′@ 85C. | |
| 43 | Maxima H | 6 | — | 1 mM GMP | 10 | 90′@ 42C., 10X | |
| | minus | | | | | (2′@50C., 2′@42C.), | |
| | | | | | | 5′@ 85C. | |
| 44 | Maxima H | 6 | — | 1 mM dCTP | 10 | 90′@ 42C., 10X | |
| | minus | | | | | (2′@50C., 2′@42C.), | |
| | | | | | | 5′@ 85C. | |
| 45 | Maxima H | 6 | — | | 10 | 90′@ 42C., 10X | |
| | minus | | | | | (2′@50C., 2′@42C.), | |
| | | | | | | 5′@ 85C. | |
| 46 | Maxima H | 3 | — | | 10 | 90′@ 42C., 10X | |
| | minus | | | | | (2′@50C., 2′@42C.), | |
| | | | | | | 5′@ 85C. | |
| 47 | Maxima H | 3 | — | 1 mM dGTP | 10 | 90′@ 42C., 10X | |
| | minus | | | | | (2′@50C., 2′@42C.), | |
| | | | | | | 5′@ 85C. | |
| 48 | Maxima H | 3 | — | 1 mM GMP | 10 | 90′@ 42C., 10X | |
| | minus | | | | | (2′@50C., 2′@42C.), | |
| | | | | | | 5′@ 85C. | |
| 49 | Maxima H | 5 | — | 3 mM GTP | 10 | 90′@ 42C., 10X | |
| | minus | | | | | (2′@50C., 2′@42C.), | |
| | | | | | | 5′@ 85C. | |
| 50 | Maxima H | 5 | — | 2 mM GTP | 10 | 90′@ 42C., 10X | |
| | minus | | | | | (2′@50C., 2′@42C.), | |
| | | | | | | 5′@ 85C. | |
| 51 | Maxima H | 5 | — | 1 mM GTP | 10 | 90′@ 42C., 10X | |
| | minus | | | | | (2′@50C., 2′@42C.), | |
| | | | | | | 5′@ 85C. | |
| 52 | Maxima H | 3 | — | 3 mM GTP | 10 | 90′@ 42C., 10X | |
| | minus | | | | | (2′@50C., 2′@42C.), | |
| | | | | | | 5′@ 85C. | |
| 53 | Maxima H | 3 | — | 2 mM GTP | 10 | 90′@ 42C., 10X | |
| | minus | | | | | (2′@50C., 2′@42C.), | |
| | | | | | | 5′@ 85C. | |
| 54 | Maxima H | 3 | — | 1 mM GTP | 10 | 90′@ 42C., 10X | |
| | minus | | | | | (2′@50C., 2′@42C.), | |
| | | | | | | 5′@ 85C. | |
| 55 | Maxima H | 3 | — | 2 mM dCTP | 10 | 90′@ 42C., 10X | |
| | minus | | | | | (2′@50C., 2′@42C.), | |
| | | | | | | 5′@ 85C. | |
| 56 | Maxima H | 3 | — | 1 mM dCTP | 10 | 90′@ 42C., 10X | |
| | minus | | | | | (2′@50C., 2′@42C.), | |
| | | | | | | 5′@ 85C. | |
| 57 | Maxima H | 3 | — | 1 m Betaine | 10 | 90′@ 42C., 10X | |
| | minus | | | | | (2′@50C., 2′@42C.), | |
| | | | | | | 5′@ 85C. | |
| 58 | Maxima H | 3 | — | | 6 | 90′@ 42C., 10X | |
| | minus | | | | | (2′@50C., 2′@42C.), | |
| | | | | | | 5′@ 85C. | |
| 59 | Maxima H | 3 | — | | 10 | 90′@ 42C., 10X | |
| | minus | | | | | (2′@50C., 2′@42C.), | |
| | | | | | | 5′@ 85C. | |
| 60 | Maxima H | 10 | — | 1 mM dCTP, 1 | 10 | 90′@ 42C., 10X | |
| | minus | | | mM GTP | | (2′@50C., 2′@42C.), | |
| | | | | | | 5′@ 85C. | |
| 61 | Maxima H | 10 | — | 4 mM dCTP | 10 | 90′@ 42C., 10X | |
| | minus | | | | | (2′@50C., 2′@42C.), | |
| | | | | | | 5′@ 85C. | |
| 62 | Superscript2 | 10 | — | 1 mM dCTP, 1 | 10 | 90′@ 42C., 10X | |
| | | | | mM GTP | | (2′@50C., 2′@42C.), | |
| | | | | | | 15′@ 70C. | |
| 63 | Superscript2 | 10 | — | 4 mM dCTP | 10 | 90′@ 42C., 10X | |
| | | | | | | (2′@50C., 2′@42C.), | |
| | | | | | | 15′@ 70C. | |
| 64 | Superscript2 | 10 | — | 1 m Betaine, 4 | 10 | 90′@ 42C., 10X | |
| | | | | mM dCTP | | (2′@50C., 2′@42C.), | |
| | | | | | | 15′@ 70C. | |
| 65 | Superscript2 | 9 | — | 1 m Betaine | 10 | 90′@ 42C., 10X | |
| | | | | | | (2′@50C., 2′@42C.), | |
| | | | | | | 15′@ 70C. | |
| | PCR | | PCR | PCR vol | | μL | additional |
| Condition | enzyme | PCR protocol | cycles | (μL) | Purification | Elution | descripion |
| 1 | KAPA (0.4x) | 3′ @ 98C., X (20″ @ | 20 | 10 | yes | 10 | 0.4x KAPAbuffer, |
| | | 98C., 30″ @ 65C., 6′ | | | | | 30 mM Tris. 50 mM |
| | | @72C.), 5′ @ 72C. | | | | | NaCl, 8 mM dTT, |
| | | | | | | | 0.05U/μL DNA |
| | | | | | | | polymerase |
| 2 | KAPA | 3′ @ 98C., X (20″ @ | 20 | 25 | yes | 15 | Longer ISO (34 bp) |
| | | 98C., 30″ @ 65C., 6′ | | | | | |
| | | @72C.), 5′ @ 72C. | | | | | |
| 3 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 4 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 5 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 6 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 7 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 8 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 9 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 10 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 11 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 12 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 13 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 14 | KAPA | 3′ @ 98C., X (20″ @ | 20 | 25 | yes | 15 | PEG was added to |
| | | 98C., 30″ @ 65C., 6′ | | | | | the Lysis Step |
| | | @72C.), 5′ @ 72C. | | | | | |
| 15 | KAPA | 3′ @ 98C., X (20″ @ | 20 | 25 | yes | 15 | PEG was added to |
| | | 98C., 30″ @ 65C., 6′ | | | | | the Lysis Step |
| | | @72C.), 5′ @ 72C. | | | | | |
| 16 | KAPA | 3′ @ 98C., X (20″ @ | 20 | 25 | yes | 15 | PEG was added to |
| | | 98C., 30″ @ 65C., 6′ | | | | | the Lysis Step |
| | | @72C.), 5′ @ 72C. | | | | | |
| 17 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 18 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 19 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 20 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 21 | KAPA | 3′ @ 98C., X (20″ @ | 20 | 25 | yes | 15 | 50 mM NaCl, 10 |
| | | 98C., 30″ @ 65C., 6′ | | | | | mM (NH₄)₂SO₂ |
| | | @72C.), 5′ @ 72C. | | | | | |
| 22 | KAPA | 3′ @ 98C., X (20″ @ | 20 | 25 | yes | 15 | 50 mM NaCl, 10 |
| | | 98C., 30″ @ 65C., 6′ | | | | | mM (NH₄)₂SO₂ |
| | | @72C.), 5′ @ 72C. | | | | | |
| 23 | KAPA | 3′ @ 98C., X (20″ @ | 20 | 25 | yes | 15 | 50 mM NaCl, 10 |
| | | 98C., 30″ @ 65C., 6′ | | | | | mM (NH₄)₂SO₂ |
| | | @72C.), 5′ @ 72C. | | | | | |
| 24 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 25 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 26 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 27 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 28 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 29 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 30 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 31 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 32 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 33 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 34 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 35 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 36 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 37 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 38 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 39 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 40 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 41 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 42 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 43 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 44 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 45 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 46 | KAPA | 3′ @ 98C., X (20″ @ | 20 | 25 | yes | 15 | 0.5 mM dNTP/each |
| | | 98C., 30″ @ 65C., 6′ | | | | | in Lysis, 1 |
| | | @72C.), 5′ @ 72C. | | | | | mMdNTP/each |
| | | | | | | | added to RT |
| 47 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 48 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 49 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 50 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 51 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 52 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 53 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 54 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 55 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 56 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 57 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 58 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 59 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 60 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 61 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 62 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 63 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 64 | KAPA | 3′ @ 98C., X (20″ @ | 20 | 25 | yes | 15 | |
| | | 98C., 30″ @ 65C., 6′ | | | | | |
| | | @72C.), 5′ @ 72C. | | | | | |
| 65 | KAPA | 3′ @ 98C., X (20″ @ | 20 | 25 | yes | 15 | Smartseq2 [11] |
| | | 98C., 30″ @ 65C., 6′ | | | | | |
| | | @72C.), 5′ @ 72C. | | | | | |

B. Results and Discussion

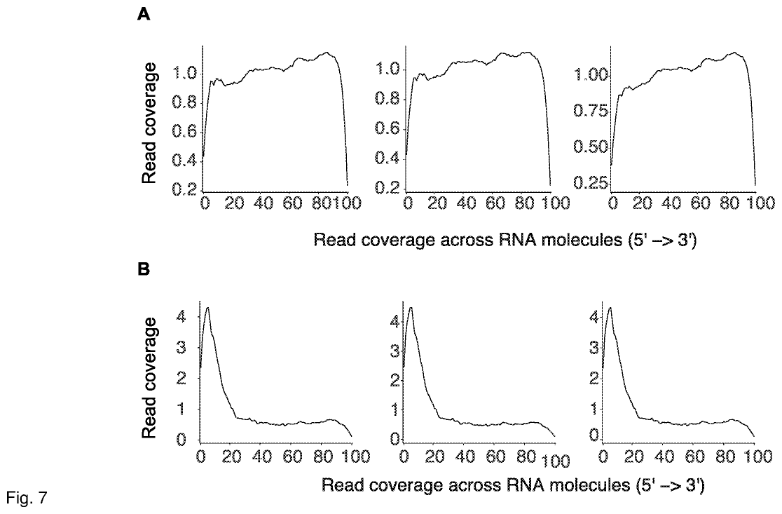

To enable single cell RNA sequencing of both full-length transcriptome information and UMIs for RNA molecule quantification, a new single cell RNA sequencing assay was designed with Smart-seq2 as a starting point. First, new oligonucleotides for reverse transcription, template switching and pre-amplification were designed (FIGS. 1A-1B). To this end, we first experimented with the template switching oligonucleotides (TSOs) that were modified to contain a partial Nextera P5 adapter sequence, a unique identification tag sequence and an UMI consisting of Ns or Hs nucleotides, as defined by International Union of Pure and Applied Chemistry (IUPAC). The oligo-dT oligonucleotides were modified in terms of length of T-stretch and end modifications. Pre-amplification PCR primers were modified to incorporate the remaining Nextera P5 adapter sequence onto the 5′ end of the captured cDNA. This allowed for sequencing of both 5′ end cDNA fragments carrying the unique identification tag and UMI, as well as fragments of the full length transcript (FIGS. 7A-7B). The complete workflow is presented in FIGS. 1A-1B.

Based on this general design, a large number of TSOs (Table 2), oligo-dT oligonucleotides (Table 1) and PCR oligonucleotides (Table 3) were experimentally tested. The new oligonucleotide designs were evaluated based on their ability to capture RNA and amplify cDNA from HEK393T cells that were individually sorted into 96 or 384 well plates. The cDNA products of the oligonucleotide designs that resulted in high amplified cDNA yield and length were tagmented and prepared for sequencing and used in subsequent experiments. A large number of reaction conditions and additives were systematically investigated for their ability to increase the capture and conversion of RNA to cDNA. An ILLUMINA® NextSeq 500 sequencing system was used to monitor the transcriptome complexity captured per cell, quantified in terms of number of genes detected per cell and the number of unique UMIs detected per cell (after excluding UMI sequences due to sequencing errors and those within one hamming distance of another UMI). Significantly improved sensitivity was obtained as compared to existing single cell RNA sequencing assays, including Smart-seq2. Several reverse transcriptase enzymes improved processivity and thermal tolerance over SuperscriptII. For instance, the reverse transcriptase Maxima H minus was used in a new reaction buffer that together improved the gene capture and sensitivity at significantly reduced cost. For the reverse transcriptase reaction, the amount of dNTPs (0.1 mM/each-0.8 mM/each) and the MgCl₂ range of (2-4 mM) were reduced, which, in the context of Maxima H minus, improved the overall yield and sensitivity. To systematically evaluate the performance, 65 different variations of this general reverse transcription and template-switching reaction were tested in addition to the experimenting with various additives (see below). The number of genes detected per cell for the 65 different conditions is presented in FIG. 2. Significantly improved gene detection as compared to Smart-seq2 was observed for many of the different conditions. The improved sensitivity also resulted in the detection of more polyadenylated non-coding RNAs, most notably long intergenic noncoding RNAs (lincRNAs) (FIG. 3).

Furthermore, cDNA conversion from RNA was improved by addition of enhancing additives, in particular dCTP and GTP in the ranges of 0.1-2 mM both alone and in combination, as well as the molecular crowding agent PEG in the range 2-9%. Extra addition of dCTP could increase the incorporation rate of C in the C-tail created by the reverse transcription enzyme at the 3′ end of the synthesized cDNA strand. Furthermore, the addition of complementary ribonucleotides to the template switching reaction has been shown to promote longer or more stable non-templated C-tails, in the context of the Moloney murine leukemia virus reverse transcriptase (MMLV-RT) when it reaches the 5′-end of the RNA template. It was hypothesized that administration of complementary ribonucleotides (GTP) could be used to increase the efficiency of the template switching reaction for single-cell RNA sequencing. As demonstrated herein, addition of dCTP and GTP impacted the genes captured in the resulting single cell RNA sequencing libraries. The crowding agent PEG is believed to increase the enzymatic reaction rates and efficiency by reducing the effective reaction volume. The crowding agent PEG substantially increased the sensitivity, both as a single additive or together with other additives as GTP (FIG. 2).

To reduce the total hands-on time required for construction of the single cell RNA sequencing libraries and to facilitate its high-throughput incorporation, we also demonstrated the possibility of performing reverse transcription and PCR pre-amplification in a one-step reaction instead of as a two-steps reaction (FIG. 2).

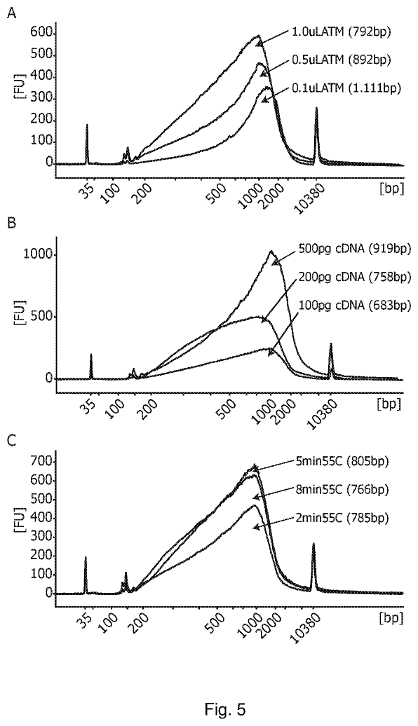

For different biological applications, it could be favorable to have a higher or lower fraction of UMI-containing 5′ reads in the final sequencing libraries. For example, experiments that utilize genomic variation in the transcriptome would need a higher number of internal reads whereas experiments that count RNAs would need higher coverage across the 5′ ends of RNAs. It was possible to experimentally control the percentage of UMI-containing 5′ reads in the sequencing libraries by tuning or modulating the tagmentation efficiency. This tuning or modulation could be performed by modifying the Tn5-to-cDNA ratio and/or by reducing the reaction time to thereby increase or decrease the percentage of UMI-containing 5′ reads in the sequencing libraries (FIG. 4). In general, the length distributions of the sequencing libraries were a strong indicator of the fraction of UMI-containing 5′ reads in the sequencing library (FIG. 5), as longer fragments were more likely to include the 5′ end. The unique ability to both capture UMIs at the 5′ end and internal RNA fragments combined with experimental strategies for controlling their relative abundances in sequencing libraries are significant advantages of the invention.

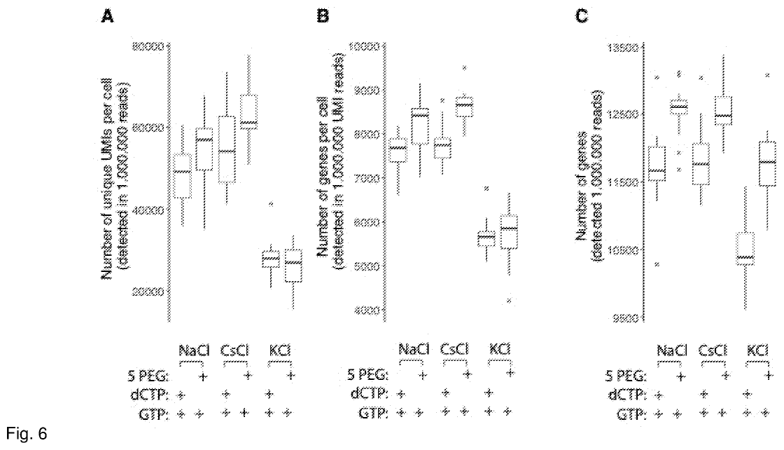

The secondary structures of RNAs have important functions and also affect the ability to reverse transcribe the RNAs into cDNAs. In single-cell RNA-sequencing applications, the utilization of NaCl or CsCl instead of KCl led to increased sensitivity of the single-cell RNA-sequencing reaction (FIG. 6). KCl promotes a four-stranded structure in the RNA molecule that include rG nucleotides, either intramolecularly or intermolecularly, the improvement observed is likely due to reduced structured RNAs that were more efficiently reverse transcribed into cDNAs and therefore captured in the resulting sequencing of the libraries. Notably, using LiCl was worse than using the standard KCl (data not shown).

FIG. 2 illustrate boxplots showing the number of genes detected per cell for each of the 65 different experimental condition tested and listed in Table 4. Condition 65 is the pre-existing Smart-seq2 libraries. A large variety of new reaction conditions using the invention detect significantly higher numbers of genes per cell as compared to Smart-seq2. The number of unique cells analyzed per condition is presented on the right side of the boxplot. The boxplot has default layout, i.e., hinges denote the first and third quartiles and whiskers denote 1.5× the interquartile range (IQR).

FIGS. 3A and 3B illustrate boxplots showing the number of genes detected per cell for a representative subset of experimental conditions tested (see Table 4) and categorized by gene biotype. Note that in addition to significantly increased detection of protein-coding RNAs, the present invention also detects significantly more non-coding RNAs including lincRNAs as compared to Smart-seq2. snoRNA in FIGS. 3A and 3B indicate small nucleolar RNA.

FIG. 4 illustrate boxplots showing the percentage 5′ end reads with UMIs within sequencing libraries for condition 11 (see Table 4) for different tagmentation reaction conditions. Lowering the amounts of Tn5 transposase present in the reaction lowers tagmentation efficiency, thereby leading to more 5′-end containing reads with UMIs. Furthermore, decreasing the amount of input cDNA or increasing the tagmentation reaction time resulted in higher tagmentation efficiency and fewer UMI-containing reads in the sequencing libraries. The starting cDNA was identical for all the conditions shown in FIG. 4 except for the conditions with variable cDNA input.

Hence, the ratio of 5′ reads with UMI relative to the internal reads can be controlled or tuned by controlling or tuning the tagmentation efficiency, such as by controlling the amount of Tn5 transposase, controlling the amount of input cDNA and/or controlling the tagmentation reaction time.

FIGS. 5A to 5C illustrate cDNA length distributions of differential tagmented cDNAs. The figures illustrate Agilent BioAnalyzer traces for the libraries shown in FIG. 4. The results shown in the figures validate the levels of UMIs in the sequencing libraries can be controlled by controlling the fragment lengths in the sequencing libraries.

FIGS. 6A to 6C illustrate that gene detection can be increased by altering reaction salts and experimental additives. FIG. 6A illustrate boxplots showing the number of unique UMIs detected per cell, FIG. 6B illustrate boxplots showing the number of genes detected by UMI-containing reads per cell and FIG. 6C illustrate boxplots showing the number of genes detected by all reads per cell. Three types of salts were tested with NaCl, CsCl and KCl as indicated below boxplots. The additives 5% PEG, dCTPs and GTPs were added to reactions as indicated below boxplots.

FIGS. 7A and 7B illustrate the read coverage across RNA molecules for internal reads and UMI-containing 5′-end reads, respectively. As is shown in the figures, the internal reads cover the RNA molecules, whereas the UMI-containing 5′ end reads are heavily biased for precisely the 5′ end of the RNA molecules.

### B. REFERENCES FOR EXAMPLE 1 AND SPECIFICATION

[1] Islam et al., Characterization of the single-cell transcriptional landscape by highly multiplex RNA-seq, Genome Research (2011) 21: 1160-1167
[2] Hashimshony et al., CEL-Seq: Single-Cell RNA-Seq by Multiplexed Linear Amplification, Cell Reports (2012), 2(3): 666-673
[3] Jaitin et al., Massively Parallel Single-Cell RNA-Seq for Marker-Free Decomposition of Tissues into Cell Types, Science (2014) 343(6172): 776-779
[4] https://www.10xgenomics.com/single-cell-technology/
[5] Rosenberg et al., Single-cell profiling of the developing mouse brain and spinal cord with split-pool barcoding, Science (2018), 360(6385): 176-182
[6] Cao et al., Comprehensive single-cell transcriptional profiling of a multicellular organism, Science (2017), 357(6352): 661-667
[7] Ramsköld et al., Full-length mRNA-Seq from single-cell levels of RNA and individual circulating tumor cells, Nature Biotechnology (2012), 30: 777-782
[8] WO 2015/02713
[9] Technology Spotlight: ILLUMINA® Sequencing https://www.iliumina.com/documents/products/techspotlights/techspotlight_sequencing.pdf (retrieved on Dec. 20, 2018)
[10] Picelli et al., Smart-seq2 for sensitive full-length transcriptome profiling in single cells, Nature Methods (2013), 10(11): 1096-1098
[11] Picelli, Full-length RNA-seq from single cells using Smart-seq2, Nature Protocols (2014), 9(1): 171-181

### II. Example 2—Single-Cell RNA Counting at Allele- and Isoform-Resolution Using Smart-Seq3

### A. Introduction

Large-scale sequencing of RNAs from individual cells can reveal patterns of gene, isoform and allelic expression across cell types and states¹. However, current single-cell RNA-sequencing (scRNA-seq) methods have limited ability to count RNAs at allele- and isoform resolution, and long-read sequencing techniques lack the depth required for large-scale applications across cells^{2,3}. Here, we introduce Smart-seq3 that combines full-length transcriptome coverage with a 5′ unique molecular identifier (UMI) RNA counting strategy that enabled in silico reconstruction of thousands of RNA molecules per cell. Importantly, a large portion of counted and reconstructed RNA molecules could be directly assigned to specific isoforms and allelic origin, and we identified significant transcript isoform regulation in mouse strains and human cell types. Moreover, Smart-seq3 showed a dramatic increase in sensitivity and typically detected thousands more genes per cell than Smart-seq2. Altogether, we developed a short-read sequencing strategy for single-cell RNA counting at isoform and allele-resolution applicable to large-scale characterization of cell types and states across tissues and organisms.

Most scRNA-seq methods count RNAs by sequencing a UMI together with a short part of the RNA (from either the 5′ or 3′ end)⁴. These RNA end-counting strategies have been effective in estimating gene expression across large numbers of cells, while controlling for PCR amplification biases, yet RNA-end sequencing has seldom provided information on transcript isoform expression or transcribed genetic variation. Moreover, many massively parallel methods suffer from rather low sensitivity (i.e. capturing only a low fraction of RNAs present in cells)⁵. In contrast, Smart-seq2 has combined higher sensitivity and full-length coverage, which e.g. enabled allele-resolved expression analyses⁷, however at a lower throughput, higher cost and without the incorporation of UMIs. Sequencing of full-length transcripts using long-read sequencing technologies could directly quantify allele and isoform level expression, yet their current depths hinder their broad application across cells, tissue and organisms^{2,3}. To overcome these shortcomings, we sought to develop a sensitive short-read sequencing method that would extend the RNA counting paradigm to directly assign individual RNA molecules to isoforms and allelic origin in single cells.

### B. Materials and Methods

Cell cultures. HEK293FT cells (Invitrogen) were cultured in complete DMEM medium containing 4.5 g/L glucose and 6 mM L-glutamine (Gibco), supplemented with 10% Fetal Bovine Serum (Sigma-Aldrich), 0.1 mM MEM Non-essential Amino Acids (Gibco), 1 mM Sodium Pyruvate (Gibco) and 100 μg/mL Pencillin/Streptomycin (Gibco). Cells were dissociated using TrypLE express (Gibco) and stained with Propidium Iodide, to exclude dead cells, before distribution into 96 or 384 well plates containing 3 μL lysis buffer using a BD FACSMelody 100 μm nozzle (BD Bioscience). The Smart-seq3 lysis buffer consisted of 0.5 unit/μL Recombinant RNase Inhibitor (RRI) (Takara), 0.15% Triton X-100 (Sigma), 0.5 mM dNTP/each (Thermo Scientific), 1 μM Smart-seq3 oligo-dT primer (5′-Biotin-ACGAGCATCAGCAGCATACGA T₃₀VN-3′ (SEQ ID NO:77); IDT), 5% PEG (Sigma) and 0.05 μL of 1:40.000 diluted ERCC spike-in mix 1 (For HEK293FT cells). The plates were spun down immediately after sorting and stored at −80 degrees.

Primary mouse fibroblasts were obtained from tail explants of CAST/EiJ×C57/BI6J derived adult mice (with ethical approval from the Swedish Board of Agriculture, Jordbruksverket: N343/12). Cells were cultured and passaged twice in (DMEM high glucose (Invitrogen), 10% ES cell FBS (Gibco), 1% Penicillin/Streptomycin (Invitrogen), 1% Non-essential amino acids (Invitrogen), 1% Sodium-Pyruvate (Invitrogen), 0.1 mM b-Mercaptoethanol (Sigma), before stained with Propidium Iodide, and sorted in to 384 well plates containing 3 μL Smart-seq3 lysis buffer. Again, plates were spun down and stored at −80 degrees immediately after sorting.

The Human Cell Atlas (HCA) reference sample consisting of a mix of Human PBMCs, Mouse colon, as well as fluorescent labelled cell-lines HEK-293-RFP, NiH3T3-GFP and MDCK-Turbo650 were thawed according to specified instructions⁴. Cells were stained with Live/Dead fixable Green Dead cell stain kit (Invitrogen), facilitating the exclusion of dead cells as well as NIH3T3-GFP cells. Additionally, both debris and doublets were excluded in the gating. Cells were index sorted into 384 well plates, containing 3 μL Smart-seq3 lysis buffer, using a BD FACSMelody sorter with 100 μm nozzle (BD Bioscience).

Generation of Smart-seq2 libraries. Smart-seq2 cDNA libraries were generated according the published protocol²². For Smart-seq2-UMI, cDNA libraries were generated as previously published¹². Recipes for other “intermediate” Smart-seq2 reactions can be found in Table 4. Tagmentation was performed with similar cDNA input and volumes as for Smart-seq3 described below.

Generation of Smart-seq3 libraries. To facilitate cell lysis and denaturation of the RNA, plates were incubated at 72 degrees for 10 min, and immediately placed on ice afterwards. Next, 1 μL of reverse transcription mix, containing 25 mM Tris-HCL pH 8.3 (Sigma), 30 mM NaCl (Ambion), 1 mM GTP (Thermo Scientific), 2.5 mM MgCl₂ (Ambion), 8 mM DTT (Thermo Scientific), 0.5 u/μL RRI (Takara), 2 μM of different Smart-seq3 Template switching oligo (TSO) (see additional table for list of evaluated TSOs; 5′-Biotin-AGAGACAGATTGCGCAATGNNNNNNNNrGrGrG-3′ (SEQ ID NO:78); IDT) and 2 u/μL Maxima H-minus reverse transcriptase enzyme (Thermo Scientific), were added to each sample. Reverse transcription and template switching were carried out at 42 degrees for 90 min followed by 10 cycles of 50 degrees for 2 min and 42 degrees for 2 min. The reaction was terminated by incubating at 85 degrees for 5 min. PCR preamplification was performed directly after reverse transcription by adding 6 μL of PCR mix, bringing reaction concentrations to 1×KAPA HiFi PCR buffer (contains 2 mM MgCl₂ at 1×) (Roche), 0.02 u/μl DNA polymerase (Roche), 0.3 mM dNTPs, 0.1 μM Smartseq3 Forward PCR primer (5′-TCGTCGGCAGCGTCAGATGTGTATAAGAGACAGATTGCGCAATG-3′ (SEQ ID NO:79); IDT), 0.1 μM Smartseq3 Reverse PCR primer (5′-ACGAGCATCAGCAGCATACGA-3′ (SEQ ID NO:80); IDT). PCR was cycled as follows: 3 min at 98 degrees for initial denaturation, 20-24 cycles of 20 secs at 98 degrees, 30 sec at 65 degrees, 6 min at 72 degrees. Final elongation was performed for 5 min at 72 degrees. For various iterations and optimization conditions, see Supplementary table 1 for information about specific conditional changes to library preparation.

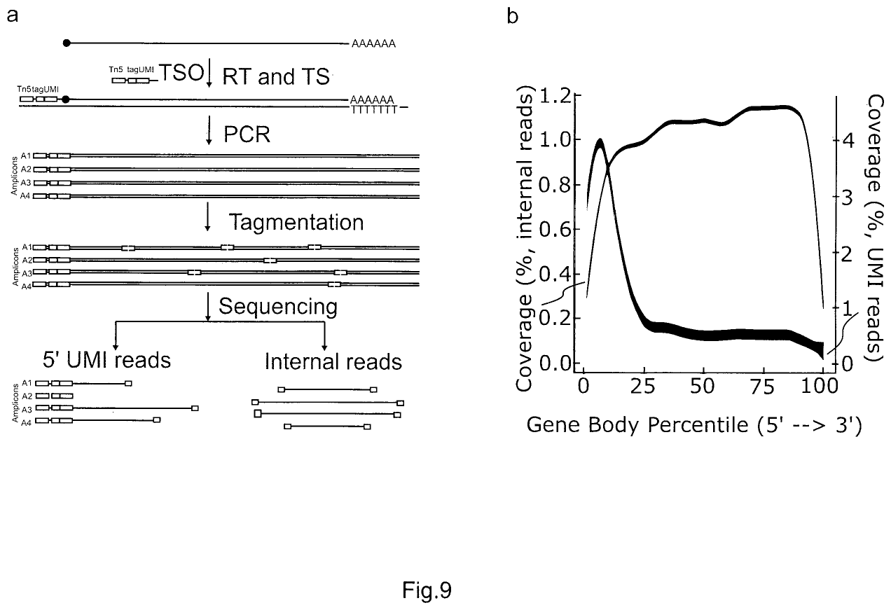

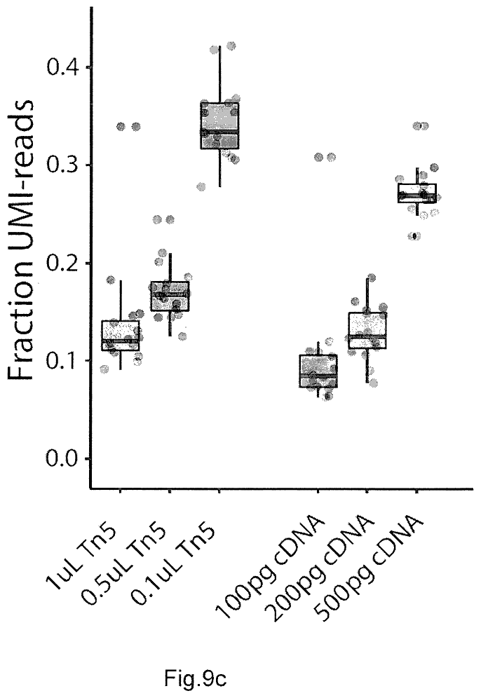

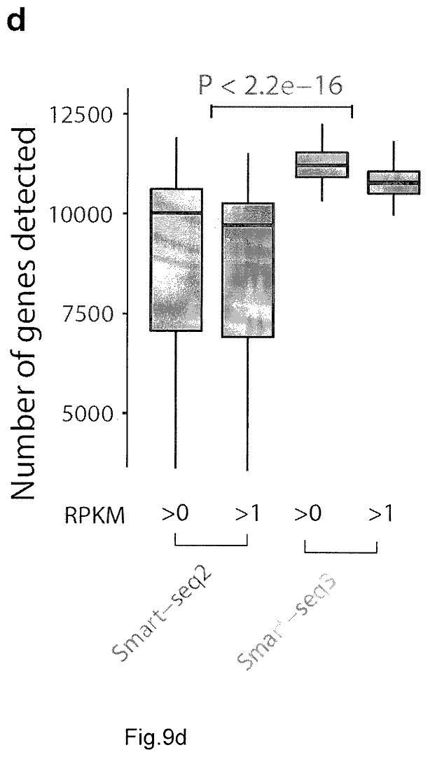

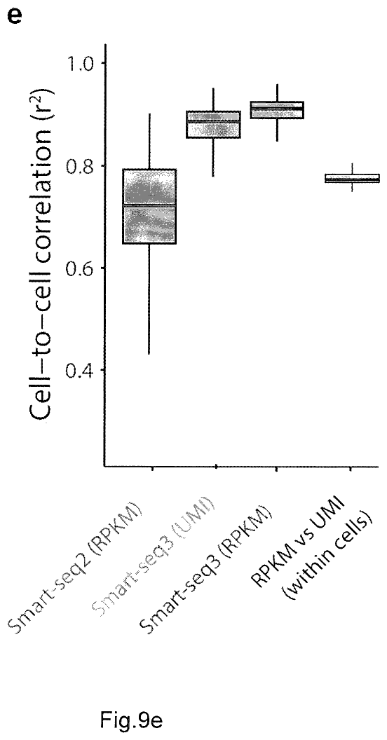

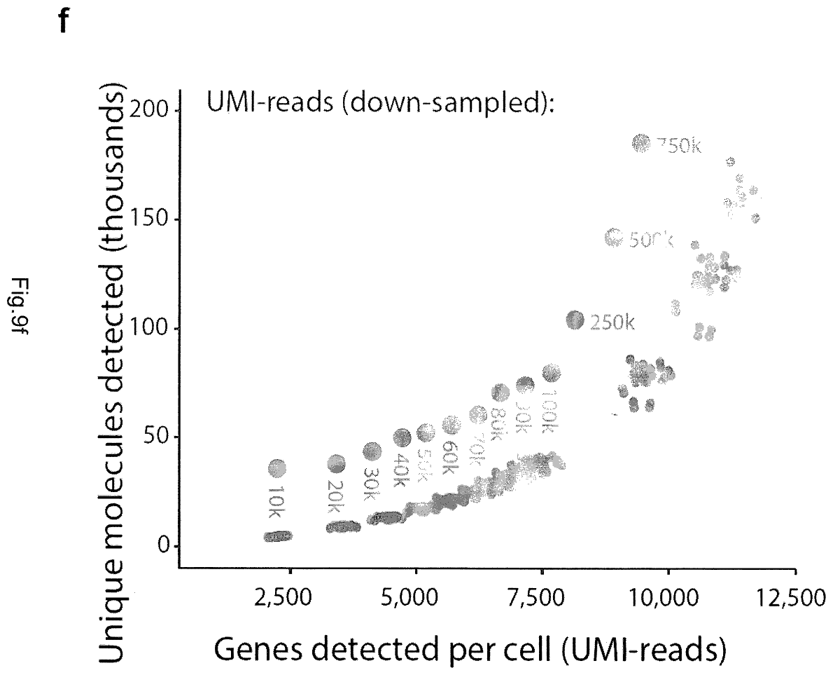

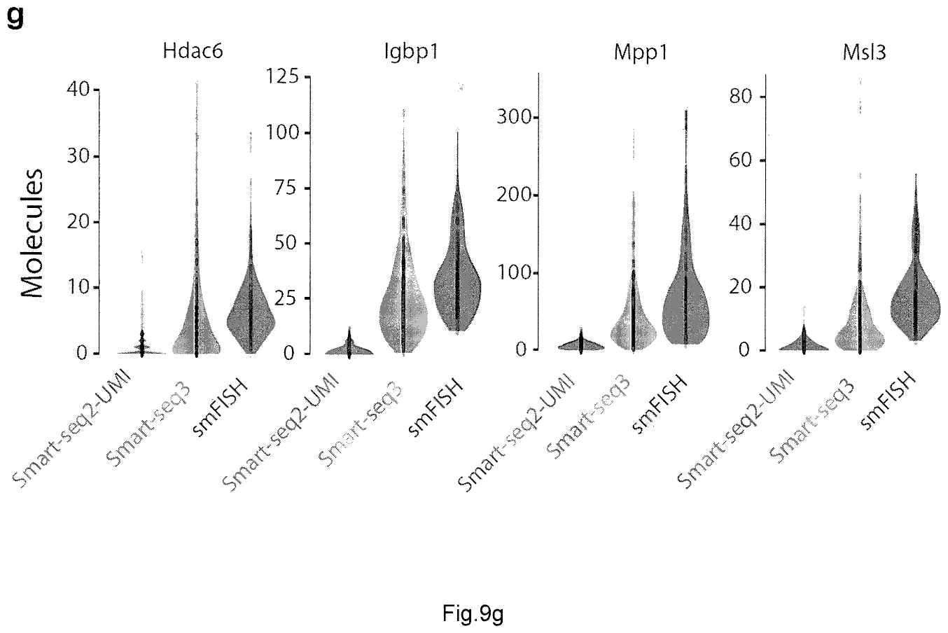

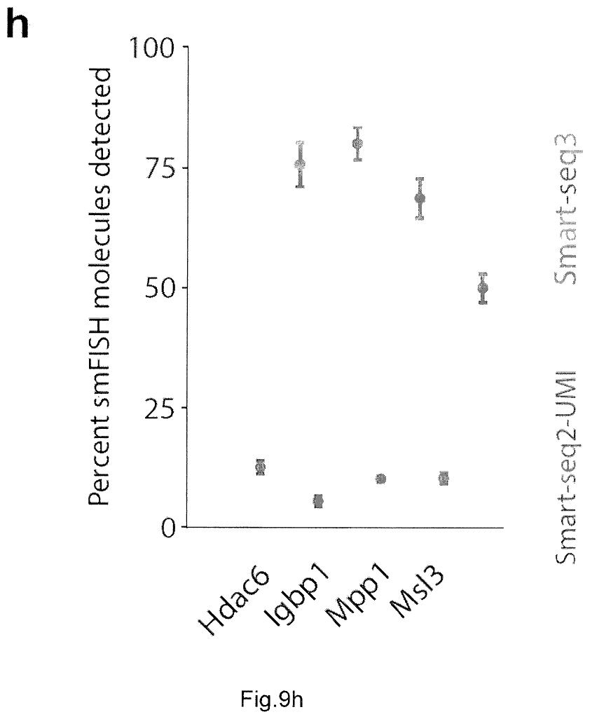

Sequence library preparation. Following PCR preamplification, all samples, regardless of protocol used, were purified with either AMpure XP beads (Beckman Coulter) or home-made 22% PEG beads (see step 27 in protocol doi:10.17504/protocols.io.p9kdr4w at protocols.io). Library size distributions were checked on a High sensitivity DNA chip (Agilent Bioanalyzer) and all cDNA concentrations were quantified using the Quant-iT PicoGreen dsDNA Assay Kit (Thermo Scientific). cDNA was subsequently diluted to 100-200pg/uL. Tagmentation was carried out in 2 uL, consisting of 1× tagmentation buffer (10 mM Tris pH 7.5, 5 mM MgCl2, 5% DMF), 0.08-0.1 uL ATM (Illumina XT DNA sample preparation kit) or TDE1 (Illumina DNA sample preparation kit), 1 uL cDNA and H2O. Plates were incubated at 55 degrees for 10 min, followed by addition of 0.5 uL 0.2% SDS to release Tn5 from the DNA. Library amplification of the tagmented samples was performed using either 1.5 uL Nextera XT index primers (Illumina) or 1.5 uL custom designed Nextera index primers containing either 8 or 10 bp indexes (0.1 uM each), differing with a minimal levenshtein distance of 2 between any two indices. 3 uL PCR mix (1× Phusion Buffer (Thermo Scientific), 0.01 U/uL Phusion DNA polymerase (Thermo Scientific), 0.2 mM dNTP/each) was added to each well, and incubated at 3 min 72 degrees; 30 sec 95 degrees; 12 cycles of (10 sec 95 degrees; 30 sec 55 degrees; 30 sec 72 degrees); 5 min 72 degrees; in a thermal cycler. For the experiments optimizing the UMI fragment conditions, following changes to the tagmentation procedure (cDNA input, amount of ATM, and time at 55 degrees) are shown in FIG. 9c. After tagmentation samples were pooled, and the pool purified with Ampure XP beads or 22% home-made PEG beads at 1:0.6 ratio. Libraries were sequenced at 75 bp single-end, or 150 bp paired-end on a high output flow cell using the Illumina NextSeq500 instrument, or on a NovaSeq S4 flow cell 150 bp paired-end.

Gel cutting pilot. We additionally experimented with selecting for certain lengths of libraries prior to sequencing of the mouse fibroblast cells. We used 20 uL of purified sequence ready library and loaded it onto a 2% Agarose E-Gel EX and ran the gel for 12 min. We manually cut the gel in the regions corresponding to 550-2000 bp and re-purified the library using Qiagen QiaQuick gel extraction kit following the manufacturers protocol. We observed a modest improvement, however selecting for longer fragments could likely improve reconstruction lengths.

Read alignments and gene-expression estimation. Raw non-demultiplexed fastq files were processed using zUMIs (version 2.4.1 or newer) with STAR (v2.5.4b), to generate expression profiles for both the 5′ ends containing UMIs as well as combined full length and UMI data. To extract and identify the UMI-containing reads in zUMIs, find_pattern: ATTGCGCAATG (SEQ ID NO:81) was specified for file1 as well as base_definition: cDNA(23-75; Single-end), (23-150 bp, paired-end) and UMI(12-19) in the YAML file. UMIs were collapsed using a Hamming distance of 1. Human cells were mapped to hg38 genome and mouse fibroblast cells were mapped against mm10 genome with CAST SNPs masked with N to avoid mapping bias, both supplemented with additional STAR parameters “—limitSjdbInsertNsj 2000000—outFilterIntronMotifs—RemoveNoncanonicalUnannotated—clip3pAdapterSeq CTGTCTCTTATACACATCT” (SEQ ID NO:82). Experiments containing HEK293FT cells were quantified with gene annotations from Ensembl GRCh38.91. Mouse primary fibroblast data was quantified with gene annotations from Ensembl GRCm38.91.

Allele-calling of F1 mouse molecules. CAST/EiJ strain specific SNPs were obtained from the mouse genome project²³ dbSNP 142 and filtered for variants clearly observed in existing CAST/EiJ×C57/BI6J F1 data, yielding 1,882,860 high-quality SNP positions. Uniquely mapped read pairs were extracted and CIGAR values parsed using the GenomicAlignments package²⁴. Reads with coverage over known high-quality SNPs were retained and grouped by UMI sequence. Molecules with >33% of bases at SNP positions showing neither the CAST nor the C57 allele were discarded and we required >66% of observed SNP bases within molecules to show one of the two alleles to make an assignment.

Inference of transcriptional burst kinetics. Allele-resolved UMI counts were used to generate maximum likelihood inference of bursting kinetics from scRNA-seq data as described previously¹². Inference scripts are available at https://github.com/sandberg-lab/txburst. To ensure a fair comparison with the data generated in this study, we reprocessed the Smart-seq2 data deposited at the European Nucleotide Archive accession E-MTAB-7098 using zUMIs and the same SNP set as described above.

Primary data processing for mixed-species benchmarking sample. The complete dataset was mapped against a combined reference genome for human (hg38), mouse (mm10) and dog (CanFam3.1). Cells mapping clearly (>75% of reads) to the mouse or dog were removed. Remaining cells representing HEK293, PBMCs and potential low quality libraries were processed using zUMIs (version 2.5.5) and mapped against the human genome only.

Analysis of human HCA benchmark samples. First, cells were filtered for low quality libraries requiring >10,000 raw reads, >75% of reads mapped to the genome and >25% exonic fractions. Further analysis was done within v3.1 of Seurat²⁵ retaining cell with >500 genes detected (intron+exon quantification). Data was normalized (“LogNormalize”) and scaled to 10,000 as well as regressing out the total number of counts per cell. The top 2,000 variable genes were found using the “vst” method and used for PCA dimensionality reduction. The first 20 principal components were used for both SNN neighborhood construction as well as UMAP dimensionality reduction. Lastly, louvain clustering was applied (resolution=0.7) to find cell groupings. Major cell types were readily identifiable by common marker genes: CD4+ T-cells (CD4, IL7R, CD3D, CD3E, CD3G), CD8+ T-cells (CD8A, CD8B), CD14+ Monocytes (CD4, CD14, S100A12), FCGR3A+ Monocytes (FCGR3A), B-cells (MS4A1, CD19, CD79A), NK-cells (NKG7, LYZ, NCAM1) and HEK cells (high number of genes detected). Naïve T-cells were separated from activated by CCR7, SELL, CD27, IL7R and lack of FAS, TIGIT, CD69. γδ T-cells were separated from other T-cells by TRGC1, TRGC2, TRDC and lack of TRAC, TRBC1, TRBC2.

Isoform reconstruction of UMI-linking fragments from Smart-seq3. The genomic alignments of 5′ UMI containing reads and their paired reads from same fragments were generated by zUMI (version 2.4.1 or newer) with UMI and cell barcode error correction. Unique and multi-mapped reads from same molecules mapping to exonic regions were used for isoform reconstruction. The genomic positions of exons from each isoform were based on reference gene annotation from Ensembl GRCm38.91 for mouse fibroblast data and Ensembl GRCh38.95 for human HCA data. Reads mapping to same molecule were compared to annotated transcripts structures, and represented as a Boolean string indicating which exon were found in read pairs and junctions (“1”) and junctions supporting the exclusion of exons (“0”). For exons not covered with reads, “N” was used to signify lacking. The Boolean string from the reconstructed molecule were matched to the string corresponding to each reference isoforms of same gene to return compatible isoform(s) for each molecule. Molecule isoform assignments were further corrected based on reads aligning to alternative 5′ and 3′ splice sites of overlapping exons from different isoforms.

Isoform assignments by integrating non-UMI reads. Transcriptome bam files generated using zUMI were demultiplexed per cell and isoform abundances quantified using Salmon¹⁵ (v0.14.0) quant command and using the following settings “—fldMean 700—fldSD 100—fldMax 2000—minAssignedFrags 1—dumpEqWeights”. We corrected the Salmon output for cases where all reads were assigned to one out of many possible isoforms belonging to the same equivalent classes. For each cell, isoforms with TPM>0 from salmon were considered expressed, and used to filter compatible isoforms of the reconstructed molecules. If more than one isoform was compatible with a reconstructed molecule (after Salmon filtering), each compatible isoform obtained a partial molecule count (1/N compatible isoforms).

Strain-specific isoform expression in mouse fibroblasts. To investigate mouse strain-specific isoform expression, we used all molecules with both an allele assigned and only a unique isoform assigned. We only considered genes for which we detected two or more isoforms and expression from both alleles. For each gene, we constructed a contingency table based on the counts of molecules assigned to each allele and isoform. Significance was tested was by using Chi-square test and the resulting p-values were corrected for the multiple testings using the Benjamini-Hochberg procedure. We further scrutinized the significant strain-isoform interactions (with an adjusted p-value <0.05). For each significant gene, we performed thousand independent randomizations of allele and isoform labels of all molecules, and we computed the Chi-square test on each permutation, and we further required that the real p-value obtained were below 5% lowest p-values from the randomizations.

### C. Results

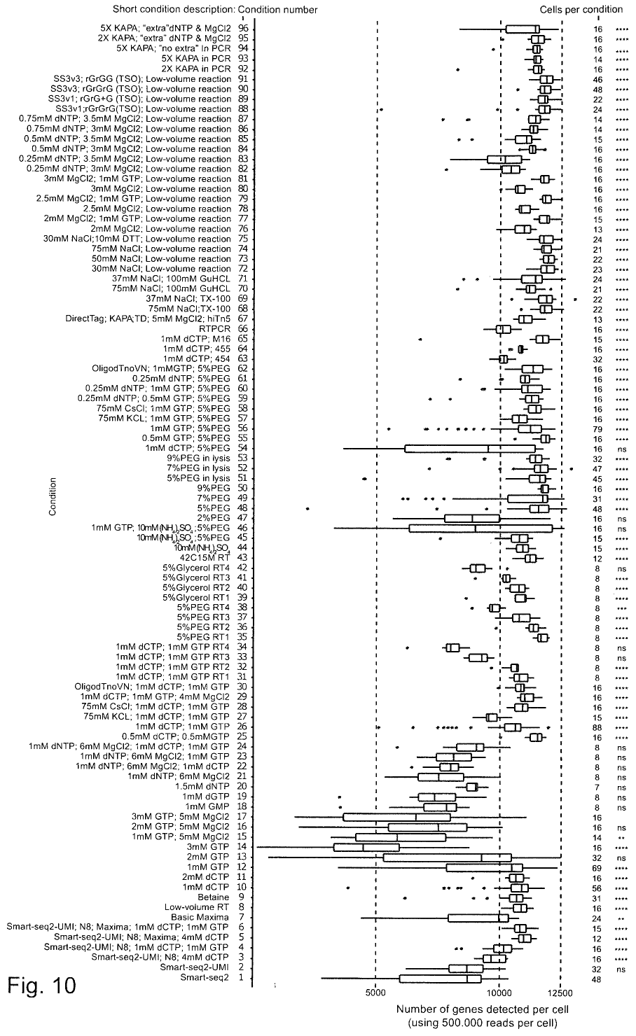

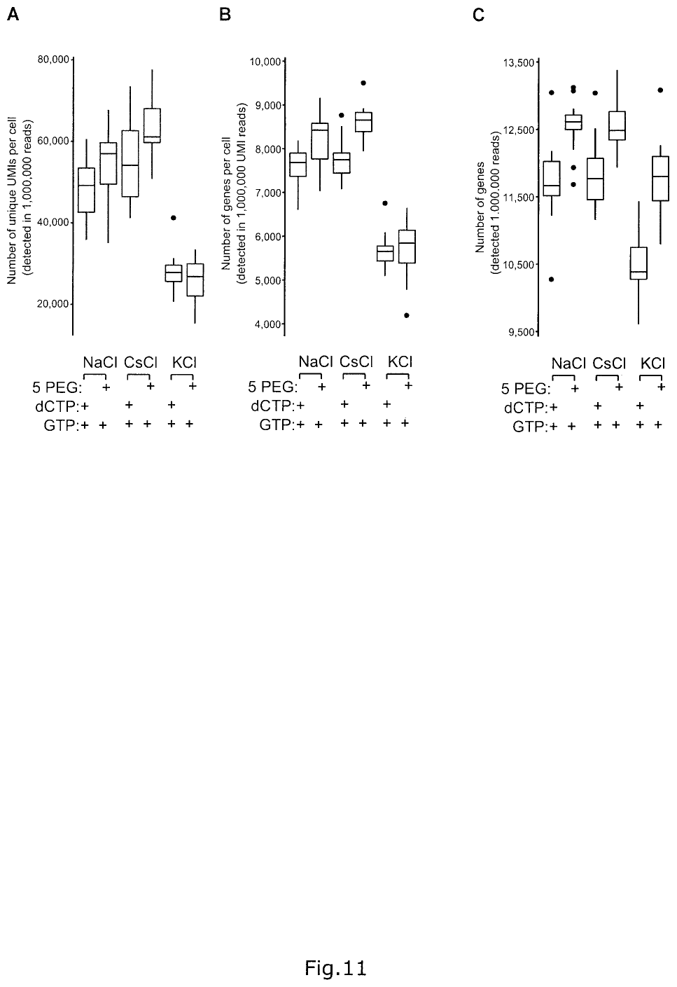

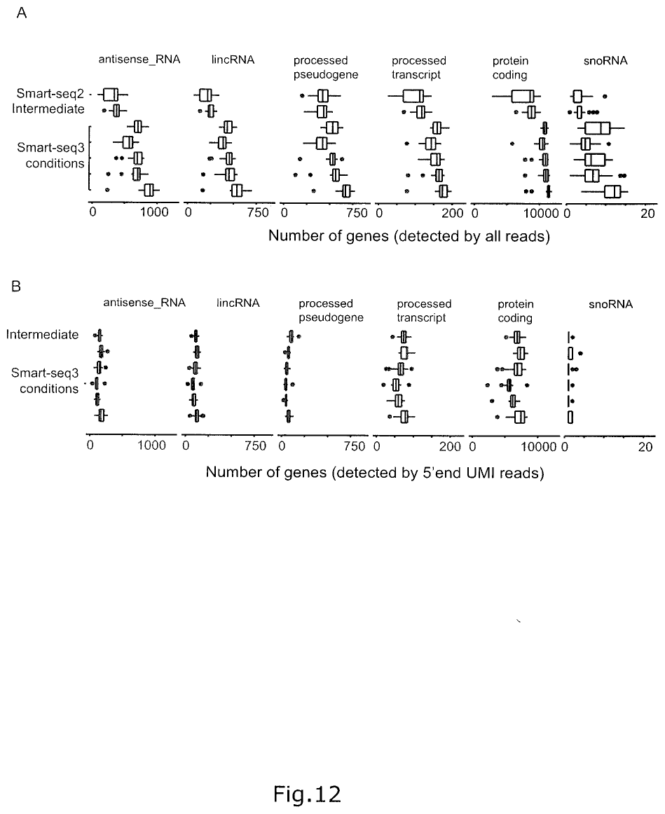

We systematically evaluated reverse transcriptases and reaction conditions that could improve the sensitivity, i.e. the number of RNA molecules detected per cell, compared to Smart-seq2⁶. Our efforts were focused on improving a Smart-seq2 like assay that retains full-length transcript coverage, thus consisting of oligo-dT priming, reverse transcription followed by template switching, full cDNA amplification using PCR and finally Tn5-based tagmentation and library construction (FIG. 9a). After assessing hundreds of different reaction conditions in HEK293T cells, with the most notable conditions sequenced (FIG. 10 and Table 4, the highest sensitivity was obtained using Maxima H-minus reverse transcriptase (hereafter called Maxima), in line with recent work⁸. We noted that switching the salt during reverse transcription from KCl to NaCl or CsCl improved sensitivity in Maxima-based single-cell reactions compared to standard KCl conditions (FIG. 11), likely due to reduced RNA secondary structures⁹. Moreover, performing reverse transcription in 5% PEG improved yields, as recently demonstrated⁸, and we added GTPs¹⁰ or dCTPs to stabilize or promote the template switching reaction (FIG. 11). We tested a number of DNA polymerase enzymes, however KAPA HiFi Hot-Start polymerase remained most compatible with the reaction chemistry and yielded highest sensitivity. Importantly, we constructed a template-switching oligo (TSO) that harbored a primer site consisting of a partial Tn5 motif¹¹ and a novel 11 bp tag sequence, followed by a 8 bp UMI sequence and three riboguanosines, the latter hybridizes to the non-templated nucleotide overhang at the end of the single-stranded cDNA. After sequencing, the 11 bp tag can be used to unambiguously distinguish 5′ UMI-containing reads from internal reads (FIG. 9a). Therefore, we obtain strand-specific 5′ UMI-containing reads and unstranded internal reads spanning the full-transcript without UMIs in the same sequencing reaction (FIG. 9b). The proportions of 5′ to internal reads could be tuned by altering the Tn5-based tagmentation reaction (FIG. 9c). We termed the final protocol Smart-seq3, and it significantly improved the detection of polyA+ protein-coding (FIG. 9d) and non-coding RNAs (FIG. 12) in HEK293FT cells. Compare to Smart-seq2, the cell-to-cell correlations in gene expression profiles improved significantly with Smart-seq3 (FIG. 9e) and we uncovered remarkable complexity in the HEK293T cell transcriptomes with up to 150,000 unique molecules detected (FIG. 9f). Strikingly, comparison of Smart-seq3 to single-molecule RNA-FISH revealed that Smart-seq3 detected up to 80% of the molecules detected by smRNA-FISH per cell¹², and on average 69% of smRNA-FISH molecules across the four genes tested (FIG. 9g,h). Altogether, this demonstrated that Smart-seq3 has significantly increased sensitivity compared to Smart-seq2 and is even approaching the sensitivity of smRNA-FISH.

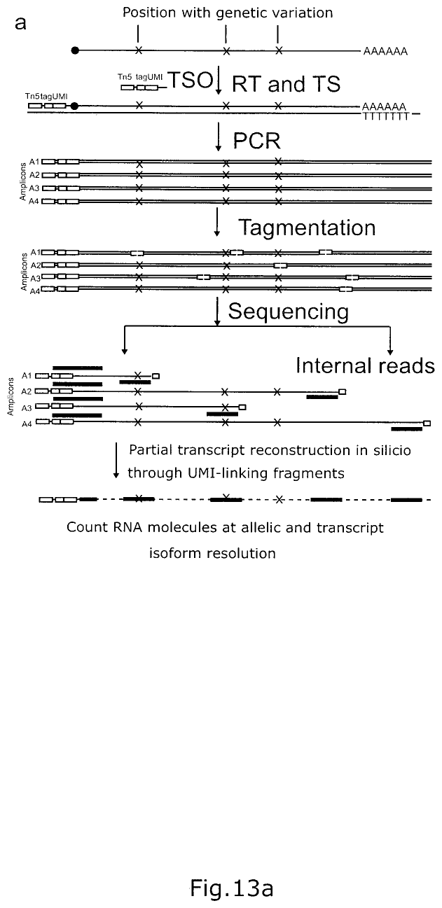

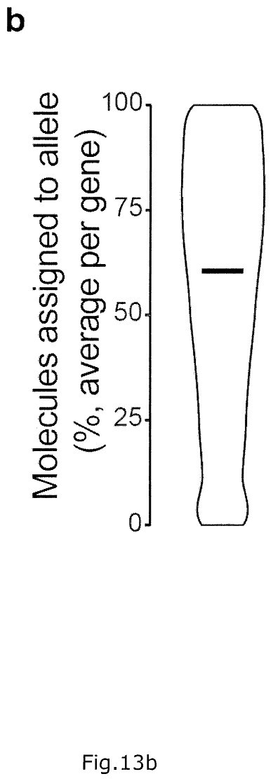

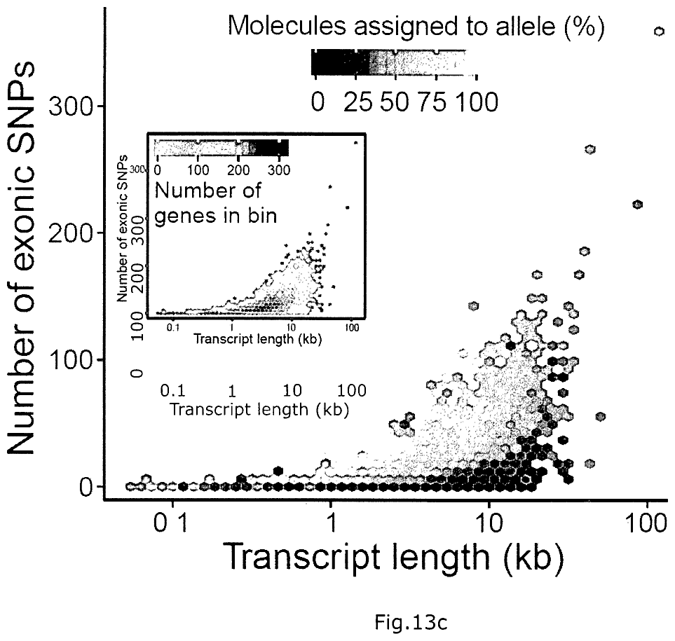

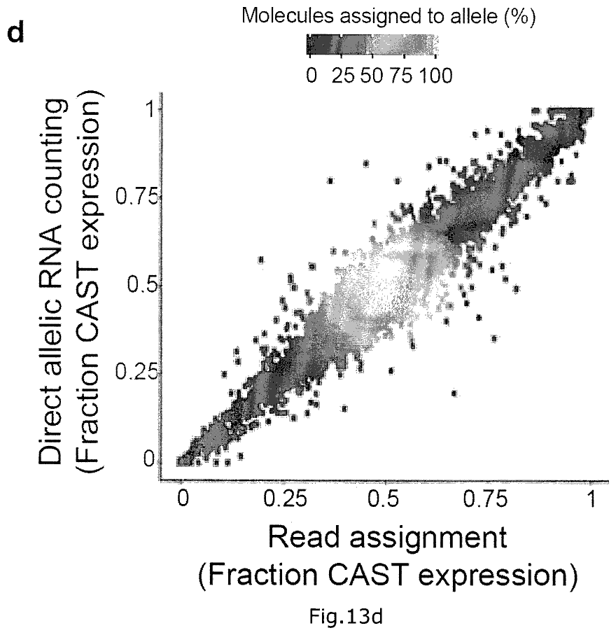

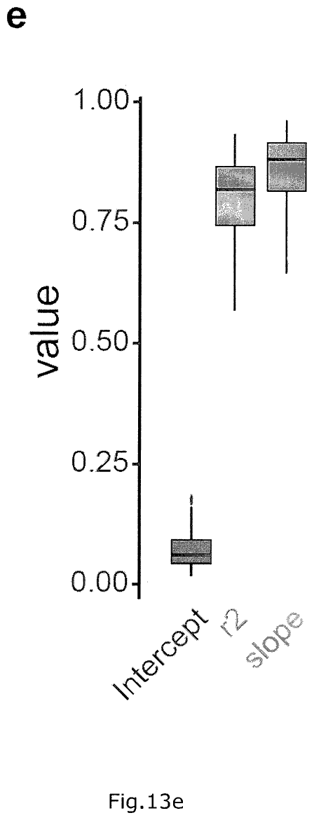

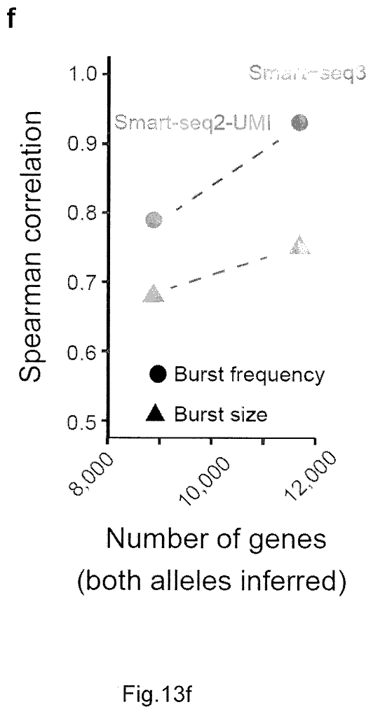

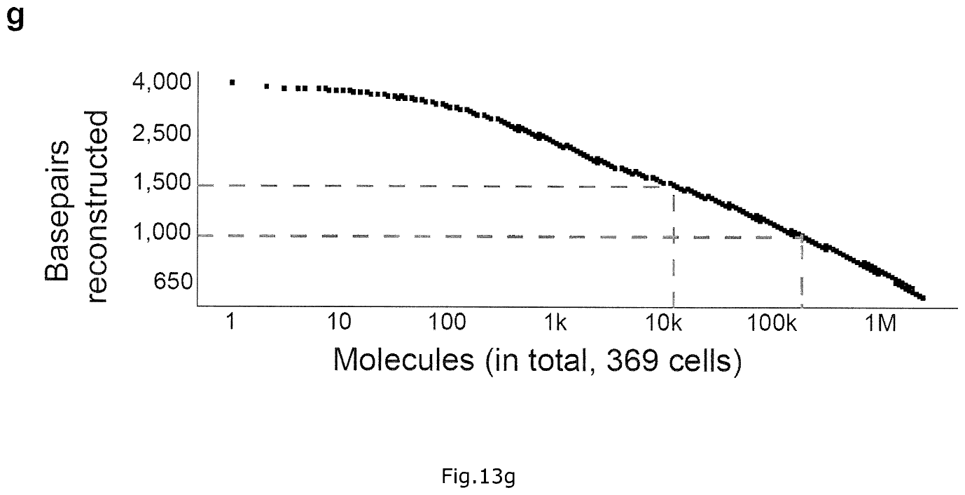

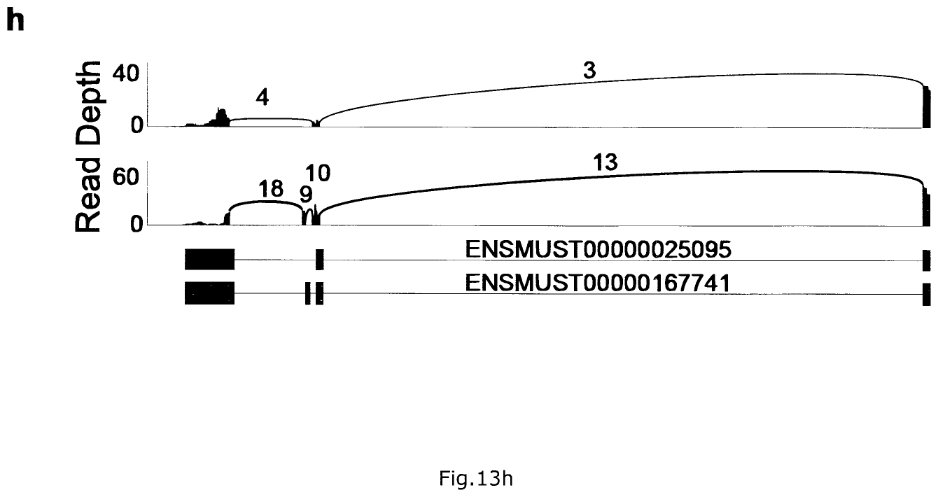

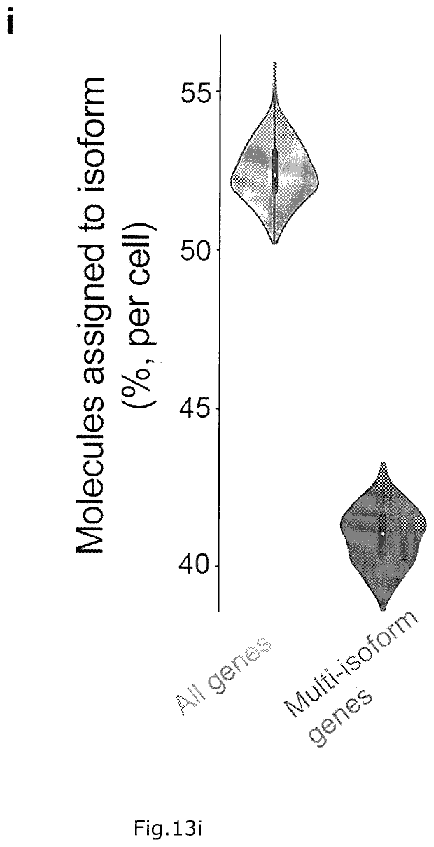

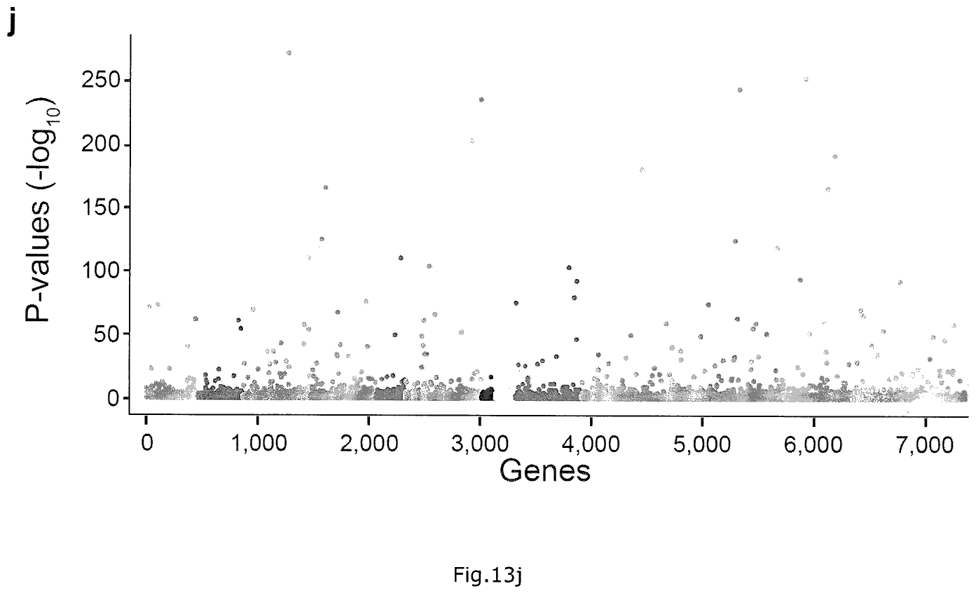

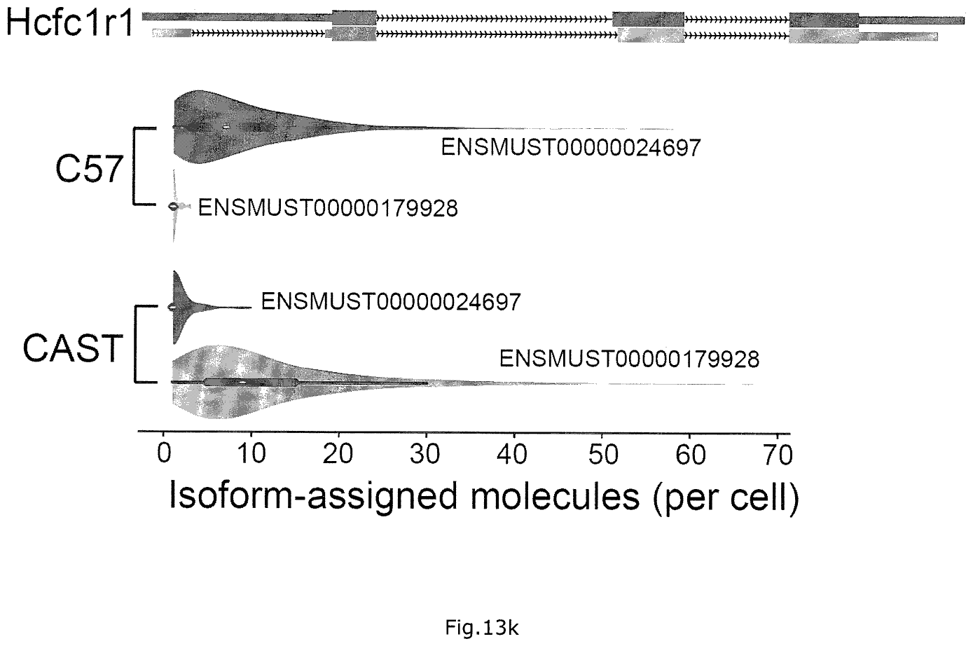

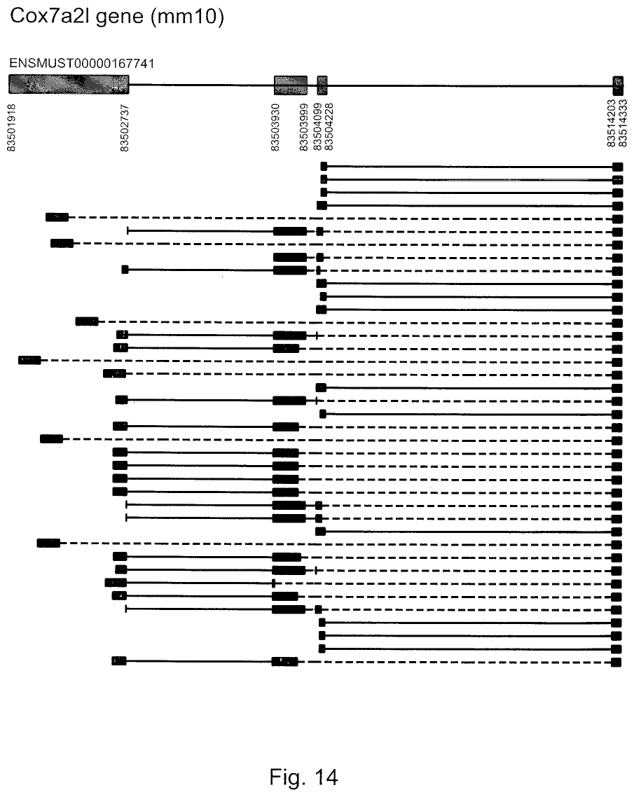

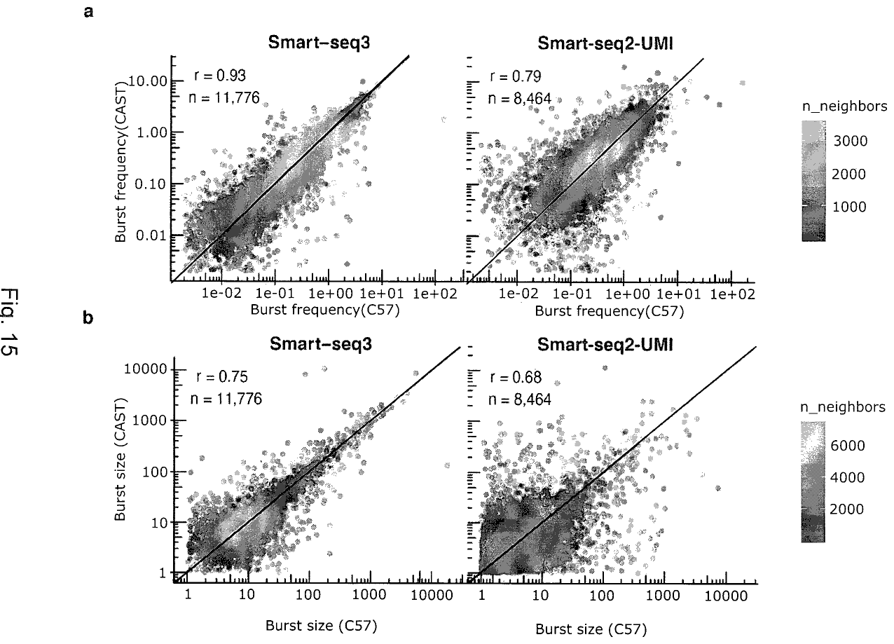

We next developed a strategy for the in silico reconstruction of RNA molecules. Importantly, the PCR preamplification of full-length cDNA in Smart-seq3 is followed by Tn5 tagmentation, so copies of the same cDNA molecule with the same UMI obtain variable 3′ ends that map to different parts of the specific transcript (FIG. 13a). Therefore, paired-end sequencing of these libraries results in 3′ end sequences that span different parts of the initial cDNA molecule that we computationally can link to the specific molecule based on the 5′ UMI sequence, thus enabling parallel reconstruction of the RNA molecules (FIG. 13a). To experimentally investigate the RNA molecule reconstructions, we created Smart-seq3 libraries from 369 individual primary mouse fibroblasts (F1 offspring from CAST/EiJ and C57/BI6J strains) that we subjected to paired-end sequencing. Aligned and UMI-error corrected read pairs¹³ were investigated and linked to molecules by their UMI and alignment start coordinates. An example of read pairs that were derived from a particular molecule transcribed from the Cox7a2I locus in a single fibroblast is visualized in FIG. 14. We then explored how often the reconstructed parts of the RNA molecules covered strain-specific single-nucleotide polymorphisms (SNPs). Strikingly, unambiguous identification of allelic origin by direct sequencing of SNPs in reads linked to the UMI was observed for 61% of all detected molecules (FIG. 13b), with increasing assignment percentage with increasing SNP density within transcripts (FIG. 13c). Previous single-cell studies estimated allelic expression as the product of the RNA quantification (in molecules or RPKMs) and fraction SNP-containing reads supporting each allele^{7,12,14}, and we next investigated how those estimates compared to the direct allelic RNA counting made possible with Smart-seq3. Reassuringly, allelic expression estimates and direct allelic RNA counting showed good overall correlation when aggregated over cells (FIG. 13d). Moreover, using a linear model to quantify the agreement of the two measures across genes within cells revealed a strong correlation (Spearman rho=0.82±0.08 and slope=0.88±0.06) without any apparent bias (intercept=0.06±0.03) (FIG. 13e). Thus, direct allelic RNA counting is feasible in single cells and validates previous efforts to estimate allelic expression from separated expression and allelic estimates in single cells^{7,12,14}.

We have previously shown that allele-resolved scRNA-seq can be used to infer bursting kinetics of gene expression that are characteristic of transcription¹². Strikingly, Smart-seq3 based analysis enabled kinetic inference for thousands more genes than using Smart-seq2 alone with a 5′ UMI (11,766 using Smart-seq3; 8,464 using Smart-seq2-UMI) and with significantly improved correlation between the CAST and C57 alleles (0.94 and 0.75 for Smart-seq3 and 0.79 and 0.68 for Smart-seq2-UMI, respectively for burst frequency and size) (FIG. 13f and FIG. 15). We conclude that Smart-seq3 enables more sensitive reconstruction of transcriptional bursting kinetics across single cells.

We investigated the lengths of RNAs reconstructed to what extent they contained information on transcript isoform structures. In our experiment with 369 cells, we observed in total 22,196 molecules reconstructed to a length of 1.5 kb or longer, and around 200,000 molecules reconstructed to 1 kb or longer (FIG. 13g). Per cell, 8,710 molecules were reconstructed to a length of 500 bp or longer. Importantly, reconstructed molecules could often be assigned to specific transcript isoforms, here exemplified by Sashimi plots for two reconstructed molecules from the Cox7a2I gene (FIG. 13h), which illustrate how reconstructed sequences overlaying exons and splice junctions could assign molecules to transcript isoforms. Intriguingly, 53% of all reconstructed molecules could be assigned to a single annotated Ensembl isoform, including 41% of all molecules detected from multi-isoform genes (FIG. 13i), thus enabling counting of RNAs at isoform resolution.

Strain-specific transcript isoform regulation has previously been hard to study, since the simultaneously quantification of strain-specific SNPs and splicing outcomes on the same RNAs have not been possible with traditional single-cell or population-level RNA-sequencing. We assigned the in silico reconstructed molecules to both allelic origin and transcript isoform structures, which revealed statistically significant strain-specific (CAST or C57) expression of transcript isoforms for 2,172 genes (adjusted p-value <0.05, chi-square test with Benjamini-Hochberg correction; and p-value <0.05, gene-specific permutation test) (FIG. 13j). For example, transcripts for Hcfc1r1 were processed into two isoforms (ENSMUST00000024697 and ENSMUST00000179928) that differed both in coding sequence (3 amino acid deletion from a 12-bp alternative 3′ splice site usage) and in 5′ untranslated region splicing. Strikingly, the two isoforms had a significant mutually exclusive pattern of expression between strains (adjusted p-value <10⁻²⁰⁸, chi-square test with Benjamini-Hochberg correction) (FIG. 13k). Thus, Smart-seq3 can simultaneous quantify genotypes and splicing outcomes, here exemplified by strain-specific splicing patterns in mouse.

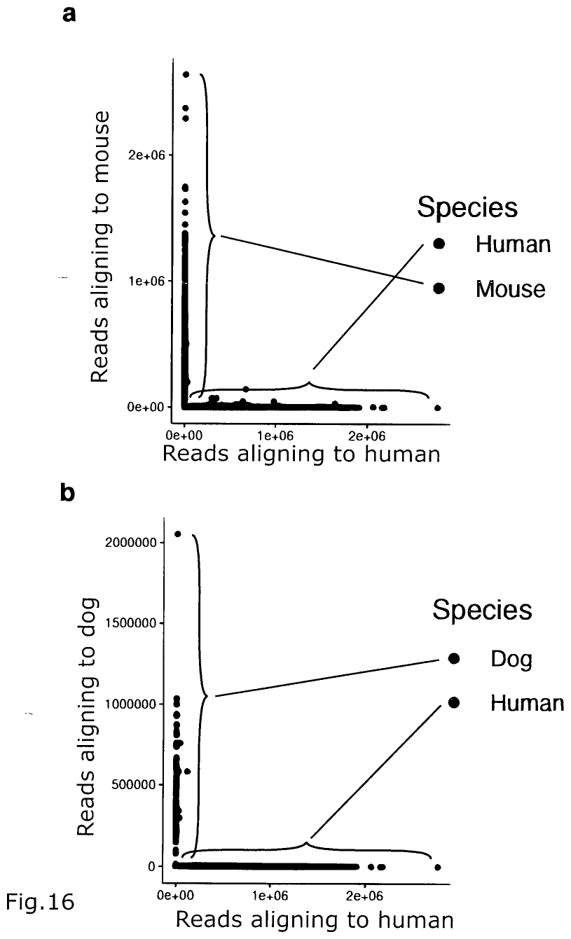

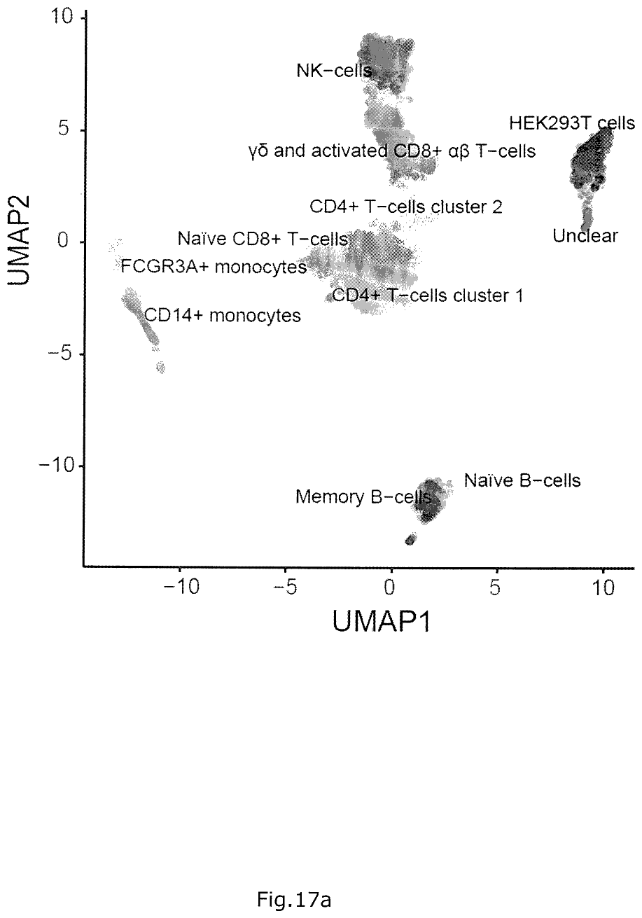

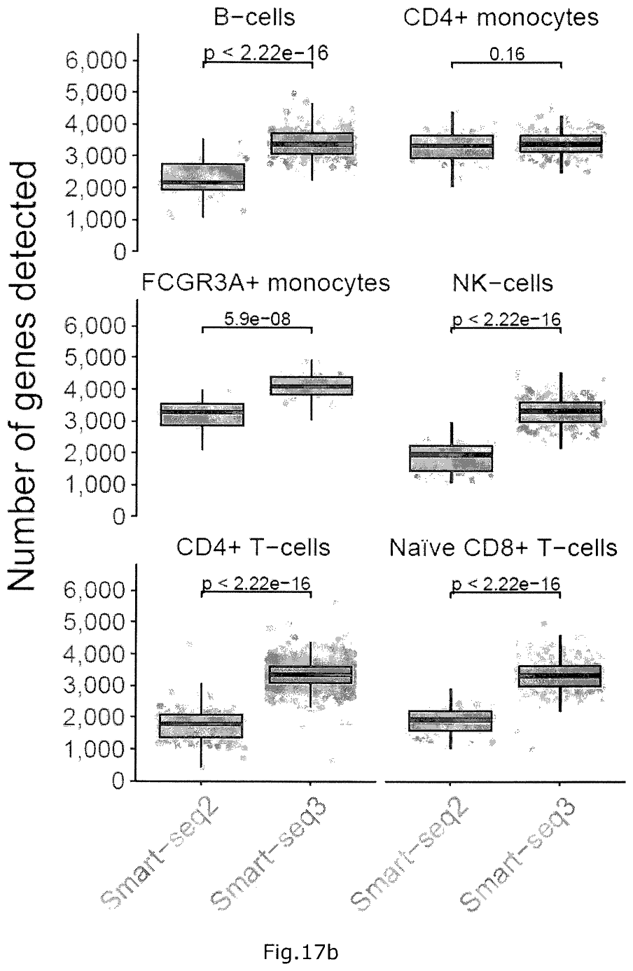

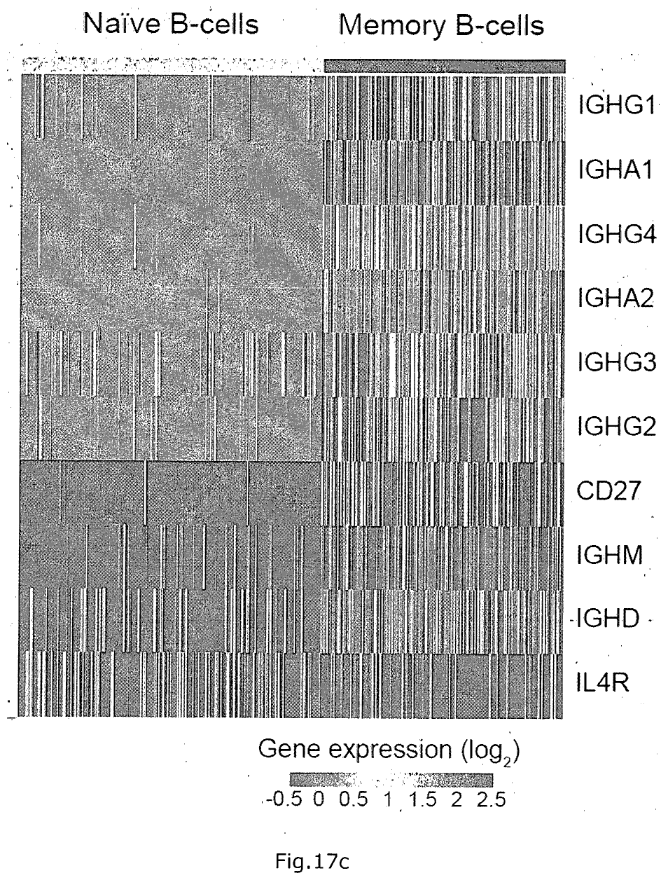

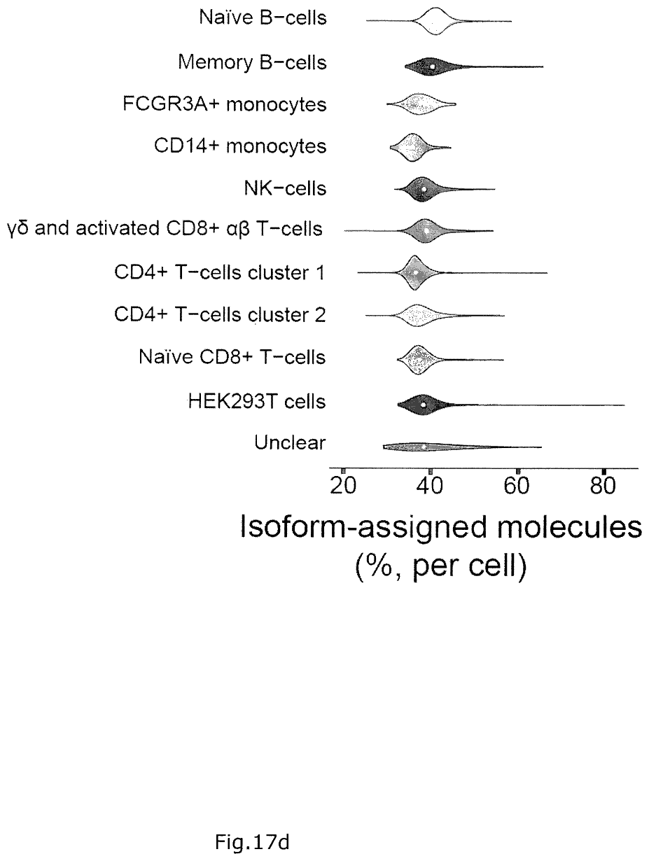

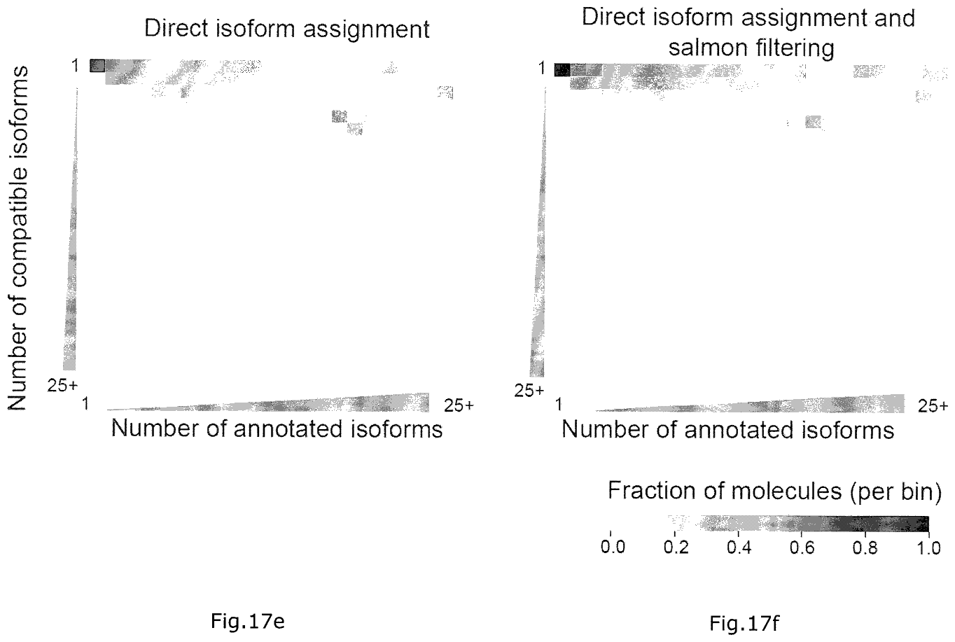

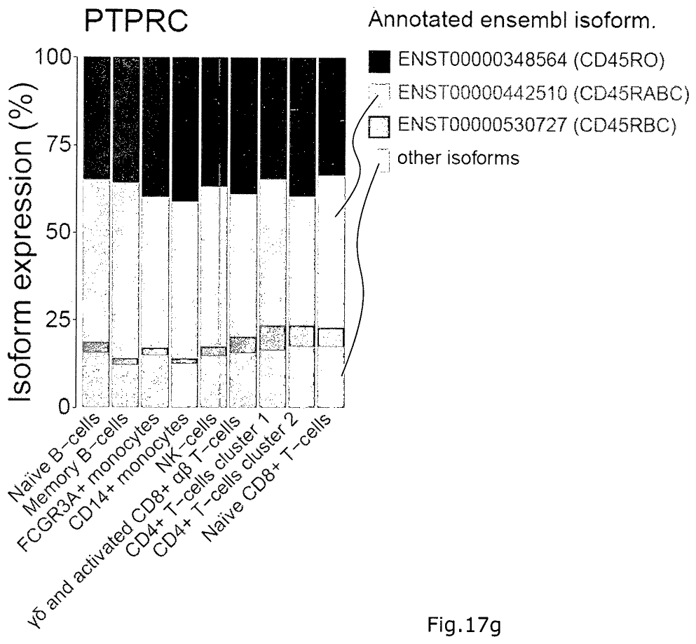

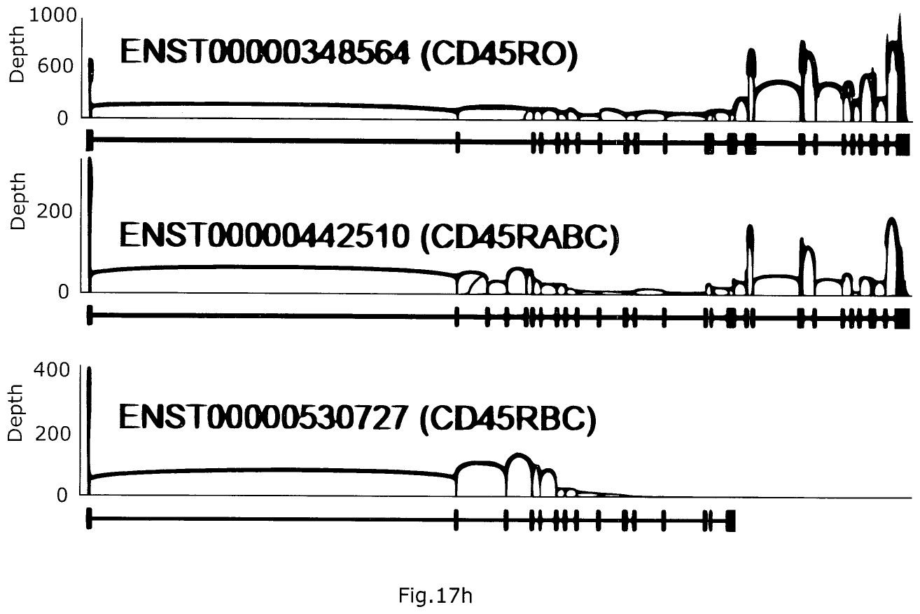

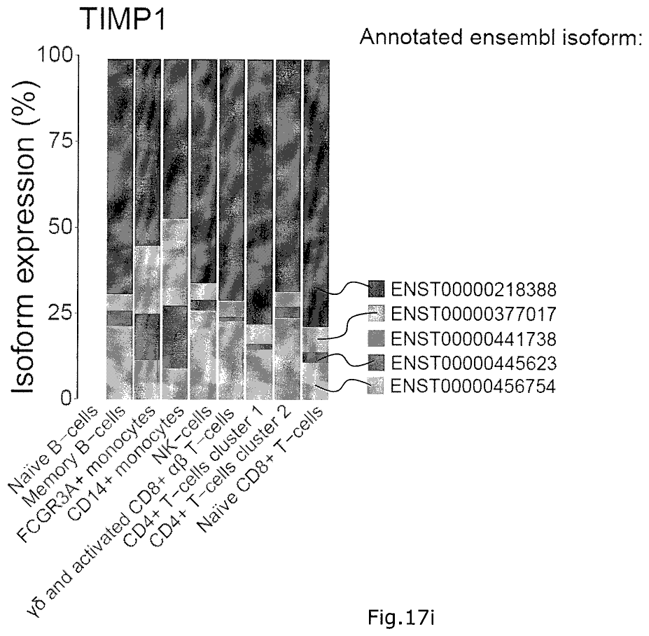

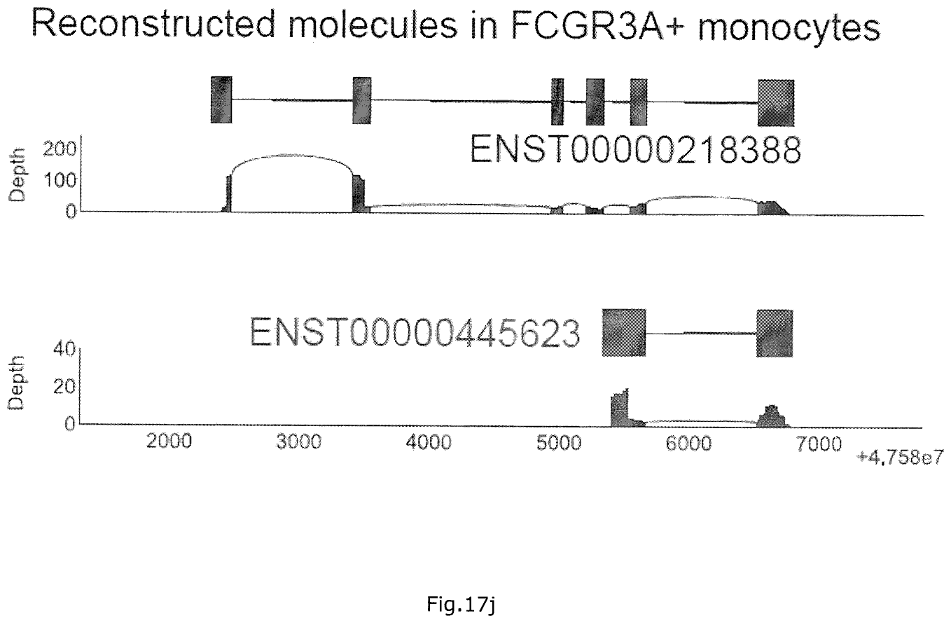

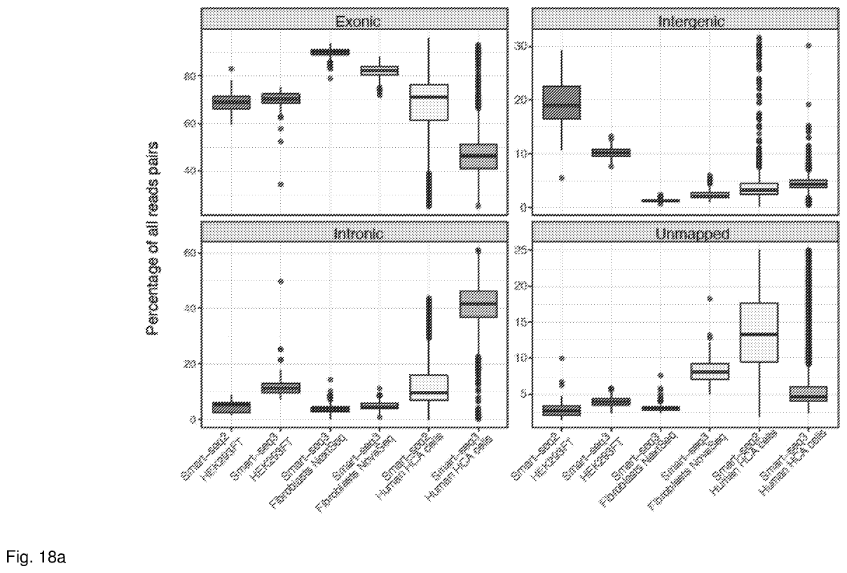

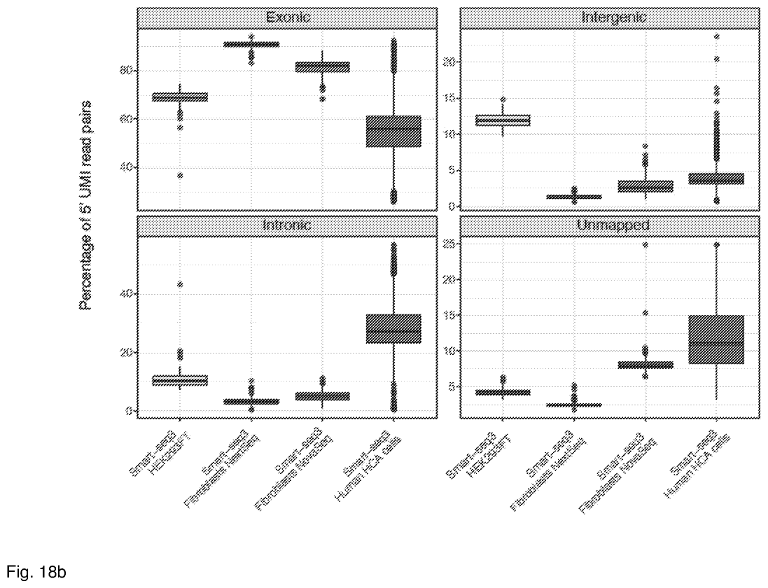

Next, we sought out to benchmark Smart-seq3 on a more complex sample consisting of many different types of cells. To this end, we sequenced 5,376 individual cells from the HCA benchmarking sample⁴, a cryopreserved and complex cell sample comprised of human peripheral blood mononuclear cells (PBMC), primary mouse colon cells and cell line spike-ins of human HEK293T, mouse NIH3T3 and dog MDCK cells. Smart-seq3 cells clearly separated according to species (FIG. 16) and cell types (FIG. 17a), and 77% of cells passed quality filtering, significantly higher percentages than the 29% to 63% reported for available protocols″, showcasing the robustness of Smart-seq3 (FIG. 18).

Except for CD14+ monocytes, which may be more vulnerable to the year-long freezer storage prior to FACS cell sorting and Smart-seq3 profiling, gene detection sensitivity was significantly higher in all cell types compared to Smart-seq2 already at shallow sequencing depths (FIG. 17b). This improvement in the number of genes detected extended into traditionally difficult cell types with low mRNA content, such as T-cells and B-cells for which we typically observed one thousand more genes per cell. Interestingly, we detected two distinct clusters of B-cells (FIG. 17a) that were not separated in single-cell data from existing methods⁴. Differential expression between the B-cell populations reported 279 genes with significant expression difference, which included several known marker genes for naïve and memory B cells (FIG. 17c). This demonstrated an improved ability of Smart-seq3 to separate biologically meaningful clusters of cells compared to existing methods.

Investigating the RNA molecule reconstruction performance across the human cell types, revealed that 36-41% of all detected molecules could be assigned to a specific isoform across cell types (FIG. 17d). To investigate the isoform assignment in greater detail, we visualized the number of compatible isoforms for each reconstructed RNA molecule, binning genes by the number of annotated isoforms. Many additional molecules could be assigned to a small set of transcript isoforms (FIG. 17e). We further reasoned that the internal reads in Smart-seq3 could provide more information on isoform expression. To this end, we computed isoform expressions using Salmon¹⁵ on all reads from Smart-seq3 and filtered the direct RNA reconstruction based assignment of molecules to only those isoforms that had detectable expression (TPM>0) in Salmon. This strategy further increased the assignment of molecules to unique isoforms (42% of all molecules) (FIG. 17f), and we used the Salmon-filtered isoform expression levels for the remainder of the study.

Next, we investigated the patterns of isoform expression across cell types. Strikingly, 2,186 genes had statistically significant patterns of isoform expressions across cell-types (Adjusted p-values <0.05; Kruskal-Wallis test and Benjamini-Hochberg correction). One of the significant genes was PTPRC (also known as CD45) which can be post-transcriptionally processed into several different isoforms¹⁶, including a full-length isoform (called RABC) and one that has excluded three consecutive exons (called RO). We mainly observed these two isoforms across the human immune cell types, although at significantly varying levels (FIG. 17g). Aggregating the reads supporting these two isoforms in gamma-delta T-cells (FIG. 17h) further shows how the reconstructed molecules separated the inclusion or skipping of the three consecutive exons. Other specific isoform patterns were shared by certain cell types, for example both CD14+ and FCGR3A+ monocytes expressed specific isoforms of the TIMP1 gene (FIG. 17i,j). Both monocyte populations specifically expressed a shorter isoform of the TIMP1 gene, whereas the long, full-length isoform was dominant across other cell types (FIG. 17i), again supported by the reconstructed molecules (FIG. 17j). Altogether, these results highlight the new and unique capabilities of using Smart-seq3 to query isoform expression and regulation across cell types.

### D. Discussion

Mammalian genes typically produce multiple transcript isoforms from each gene¹⁷, with frequent consequences on RNA and protein functions. Analysis of transcript isoform expression (in single cells or in cell populations) using short-read sequencing technologies have often focused on individual splicing events (e.g. skipped exon) or used the read coverage over shared and unique isoform regions to infer the most likely isoform expression^{18,19}. This is due to paired short reads seldom having sufficient information to assess interactions between distal splicing outcomes or combined with allelic expression from transcribed genetic variation. Long-read sequencing technologies can used to directly sequence transcript isoforms in single cells^{2,3}. However, these strategies have limited cellular throughput and depth. For example, the Mandalorion approach provided comprehensive isoform data for seven cells², whereas sclSOr-seq investigated isoform expression in thousands of cells at an average depth of 260 molecules per cell³. In contrast, we obtained on average 8,710 reconstructed molecules per cell (above 500 bp). Moreover, in sclSOr-seq the pre-amplified cDNA was sequenced on both short- and long-read sequencers in parallel to characterize cell types and sub-types, and the isoform-level sequencing data was mainly aggregated over cells according to clusters³. The use of two parallel library construction methods and sequencing technologies for the same pre-amplified cDNA from individual cells substantially increases cost and labor.

We developed Smart-seq3 to be both highly sensitive, thus improving the ability to identify cell types and states, and isoform-specific, to simultaneously reconstruct millions of partial transcripts across cells. Smart-seq3 thus removes the additional costs and labor associated with the use of multiple library preparation technologies and sequencing platforms in parallel. Compared to known transcript isoform annotations, these partial transcript reconstructions were sufficient to assign 40-50% of detected molecules to a specific isoform, which further revealed strain- and cell-type specific isoform regulation. Excitingly, this reconstruction should improve the abilities to perform splicing quantitative trait loci mapping, since both splicing outcomes and transcribed SNPs can now be directly quantified. The full Smart-seq3 protocol has been deposited at protocols.io (dx.doi.org/10.17594/protocols.io.7dnhi5e) and can be readily implemented by molecular biology laboratories without the need for specialized equipment.

Several large-scale projects aim to systematically construct cell atlases across human tissues and those of model organisms²⁰. These efforts are increasingly relying on scRNA-seq methods that count RNAs towards annotated gene ends (e.g. 10X genomics) that provides little information on isoforms expression patterns across cell types and tissues. Moreover, large-scale efforts are also emerging to use single-cell genomics for the systematic analysis of disease (e.g. the LifeTime project) to identify disease mechanisms and consequences. As post-transcriptional gene regulation has been tightly linked to disease²¹, it would be a missed opportunity for such efforts and atlases to disregard isoform-level expression patterns. In contrast to long-read sequencing efforts, Smart-seq3 simultaneously provides cost effective gene expression profiling across cell types and isoform-resolution RNA counting within the same assay. This is currently achieved at a cost per sequence ready cell library around 0.5-1 EUR. Additionally, as the current implementation uses 384-well plates, it is also possible to first shallowly sequence all cells and then later select cells of rare cell populations (as cellular amplified cDNAs can be kept in individual wells for extended periods of time) for in-depth sequencing and transcript isoform reconstruction. Altogether, we introduced a scRNA-seq method that is applicable to characterize cell types and annotate cell atlases at the level of gene, isoform and allelic expression.

### E. REFERENCES FOR EXAMPLE 2

1. Sandberg, R. Entering the era of single-cell transcriptomics in biology and medicine. Nat. Methods 11, 22-24 (2014).
2. Byrne, A. Nanopore long-read RNAseq reveals widespread transcriptional variation among the surface receptors of individual B cells. Nat. Commun. (2017).
3. Gupta, I. et al. Single-cell isoform RNA sequencing characterizes isoforms in thousands of cerebellar cells. Nat. Biotechnol. (2018) doi:10.1038/nbt.4259.
4. Mereu, E. et al. Benchmarking Single-Cell RNA Sequencing Protocols for Cell Atlas Projects. bioRxiv 630087 (2019) doi:10.1101/630087.
5. Ziegenhain, C. et al. Comparative Analysis of Single-Cell RNA Sequencing Methods. Mol. Cell 65, 631-643.e4 (2017).
6. Picelli, S. et al. Smart-seq2 for sensitive full-length transcriptome profiling in single cells. Nat. Methods 10, 1096-1098 (2013).
7. Deng, Q., Ramsköld, D., Reinius, B. & Sandberg, R. Single-cell RNA-seq reveals dynamic, random monoallelic gene expression in mammalian cells. Science 343, 193-196 (2014).
8. Bagnoli, J. W. et al. Sensitive and powerful single-cell RNA sequencing using mcSCRB-seq. Nat. Commun. 9, 2937 (2018).
9. Guo, J. U. & Bartel, D. P. RNA G-quadruplexes are globally unfolded in eukaryotic cells and depleted in bacteria. Science 353, (2016).
10. Ohtsubo, Y., Nagata, Y. & Tsuda, M. Compounds that enhance the tailing activity of Moloney murine leukemia virus reverse transcriptase. Sci. Rep. 7, 6520 (2017).
11. Cole, C., Byrne, A., Beaudin, A. E., Forsberg, E. C. & Vollmers, C. Tn5Prime, a Tn5 based 5′ capture method for single cell RNA-seq. Nucleic Acids Res. 46, e62 (2018).
12. Larsson, A. J. M. et al. Genomic encoding of transcriptional burst kinetics. Nature 565, 251-254 (2019).
13. Parekh, S., Ziegenhain, C., Vieth, B., Enard, W. & Hellmann, I. zUMIs—A fast and flexible pipeline to process RNA sequencing data with UMIs. GigaScience 7, (2018).
14. Reinius, B. et al. Analysis of allelic expression patterns in clonal somatic cells by single-cell RNA-seq. Nat. Genet. 48, 1430-1435 (2016).
15. Patro, R., Duggal, G., Love, M. I., Irizarry, R. A. & Kingsford, C. Salmon provides fast and bias-aware quantification of transcript expression. Nat. Methods 14, 417-419 (2017).
16. Martinez, N. M. & Lynch, K. W. Control of alternative splicing in immune responses: many regulators, many predictions, much still to learn. Immunol. Rev. 253, 216-236 (2013).
17. Wang, E. T. et al. Alternative isoform regulation in human tissue transcriptomes. Nature 456, 470-476 (2008).
18. Katz, Y., Wang, E. T., Airoldi, E. M. & Burge, C. B. Analysis and design of RNA sequencing experiments for identifying isoform regulation. Nat. Methods 7, 1009-1015 (2010).
19. Trapnell, C. et al. Differential analysis of gene regulation at transcript resolution with RNA-seq. Nat. Biotechnol. 31, 46-53 (2013).
20. Regev, A. et al. The Human Cell Atlas. eLife 6, (2017).
21. Scotti, M. M. & Swanson, M. S. RNA mis-splicing in disease. Nat. Rev. Genet. 17, 19-32 (2016).
22. Picelli, S. et al. Full-length RNA-seq from single cells using Smart-seq2. Nat. Protoc. 9, 171-181 (2014).
23. Keane, T. M. et al. Mouse genomic variation and its effect on phenotypes and gene regulation. Nature 477, 289-294 (2011).
24. Lawrence, M. et al. Software for computing and annotating genomic ranges. PLoS Comput. Biol. 9, e1003118 (2013).
25. Stuart, T. et al. Comprehensive Integration of Single-Cell Data. Cell 177, 1888-1902.e21 (2019).

### Example 3: Using the Method to Improve Analysis of Metagenomic Samples

Metagenomic samples can comprise nucleic acids from a wide collection of different microbial species, e.g., bacteria. A common method in the art for identifying the species present in the sample is to do amplicon-based NGS library sequencing of segments of the rRNA genes. See for example: https://genohub.com/shotgun-metagenomics-sequencing/. This method relies on the fact that the rRNA genes are generally very conserved between species and thus primers for amplicon sequencing can be designed to recognize many different species by hybridizing to the conserved (“Constant”) regions and amplifying the variable segments between them that serve to identify the species of origin. A problem in the current art is that sequencing read lengths generally only allow analysis of one of the variable regions at a time and so the ability to distinguish closely related species can be limited. It would benefit the community to have a method that could sequence longer stretches of the rRNA genes, so as to include more than one variable region. In this example, the method of the invention is applied to a metagenomic sample, where the rRNA is converted to cDNA using a gene-specific primer that hybridizes to one of the constant regions, such that a cDNA is generated the encompasses several, preferably all, of the variable regions of the rRNA and includes the copy of the TSO. This cDNA is then amplified according to the methods of the invention and fragmented and the internal and 5′ end fragments amplified to make a library as described herein. The library is then sequenced. By using the paired end reads and the ability to distinguish 5′end reads from internal reads, as described in the methods of the invention, it is possible to identify multiple variable regions belonging to the same original rRNA molecule and thus enable improved identification of the species present in the metagenomic sample from which the RNA originated.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. A method for preparing complementary deoxyribonucleic acid (cDNA) comprising:
hybridizing a cDNA synthesis primer to a ribonucleic acid (RNA) molecule and synthesizing a cDNA strand complementary to at least a portion of the RNA molecule to form an RNA-cDNA intermediate; and
performing a template switching reaction by contacting the RNA-cDNA intermediate with a template switching oligonucleotide (TSO) under conditions suitable for extension of the cDNA strand using the TSO as template to form an extended cDNA strand complementary to the at least a portion of the RNA molecule and the TSO, wherein the TSO comprises an amplification primer site, an identification tag, a unique molecular identifier (UMI) and multiple predefined nucleotides.

2. The method according to claim 1, wherein
hybridizing the cDNA synthesis primer comprises hybridizing the cDNA synthesis primer to the RNA molecule and synthesizing the cDNA strand by reverse transcription to form the RNA-cDNA intermediate; and
performing the template switching reaction comprises performing the template switching reaction by contacting the RNA-cDNA intermediate with the TSO under conditions suitable for extension of the cDNA strand by reverse transcription to form the extended cDNA strand.

3. The method according to claim 2, wherein the reverse transcription is conducted in the presence of ribonucleotides, preferably guanine ribonucleotides, at a concentration selected within an interval of from 0.05 mM to 10 mM, preferably within an interval of from 0.1 mM to 3 mM.

4. The method according to claim 2 or 3, wherein
the reverse transcription is conducted in the presence of a mixture dATP, dGTP, dTTP and dCTP;
the mixture comprises a same concentration of dATP, dGTP and dTTP and a concentration of dCTP being X mM higher than the same concentration of dATP, dGTP and dTTP; and
X is selected within an interval of from 0.05 mM to 10 mM, preferably within an interval of from 0.1 mM to 3 mM.

5. The method according to any of the claims 2 to 4, wherein the reverse transcription is conducted in the presence of a magnesium salt in a concentration selected within an interval of from 0.1 mM to 20 mM, preferably within an interval of from 1 mM to 10 mM, and more preferably within an interval of from 2 mM to 5 mM.

6. The method according to any of the claims 2 to 5, wherein the reverse transcription is conducted in the presence of a chloride salt selected from the group consisting of sodium chloride (NaCl), cesium chloride (CsCl), and a mixture thereof, and is conducted in an at least reduced amount of potassium chloride (KCl).

7. The method according to any of the claims 2 to 6, wherein the reverse transcription is conducted in the presence of a polyethylene glycol (PEG) having an average molecular weight selected within an interval of from 300 Da to 100,000 Da, preferably within an interval of from 1,000 to 25,000 Da, and more preferably within an interval of from 7,000 Da to 9,000 Da, such as 8000 Da.

8. The method according to any of the claims 1 to 7, wherein the amplification primer site comprises a portion of a transposase 5 (Tn5) motif sequence, preferably AGAGACAG.

9. The method according to any of the claims 1 to 8, wherein the identification tag comprises a nucleotide sequence that does not exist in a transcriptome of a cell from which the RNA molecule originates, preferably
(SEQ ID NO: 3)
ATTGCGCAATG.

10. The method according to any of the claims 1 to 9, wherein the multiple nucleotides are three ribonucleotides, preferably three guanine ribonucleotides.

11. The method according to any of the claims 1 to 10, wherein the cDNA synthesis primer is an oligo-dT primer, preferably an anchored oligo-dT primer, and more preferably comprises, from a 5′ end to a 3′ end, a primer site, Tₚ, V, and N, wherein V is selected from the group consisting of A, C and G, N is selected from the group consisting of A, C, G and T, and p is a positive number selected within an interval of from 10 to 50, preferably from 15 to 45, and more preferably from 20 to 40, such as 30.

12. The method according to claim 11, wherein the primer site comprises a nucleotide sequence that does not exist in a transcriptome of a cell from which the RNA molecule originates, preferably comprises
(SEQ ID NO: 5)
ACGAGCATCAGCAGCATACGA.

13. The method according to any of the claims 1 to 12, wherein
hybridizing the cDNA synthesis primer comprises hybridizing, for each RNA molecule of a plurality of RNA molecules, the cDNA synthesis primer to the RNA molecule and synthesizing a respective cDNA strand complementary to at least a portion of the RNA molecule to form a respective RNA-cDNA intermediate; and
performing the template switching reaction comprises performing the template switching reaction by contacting the respective RNA-cDNA intermediate with a respective TSO under conditions suitable for extension of the respective cDNA strand using the respective TSO as template to form a respective extended cDNA strand complementary to the at least a portion of the RNA molecule and the respective TSO, wherein each TSO comprises the amplification primer site, the identification tag, a UMI and the multiple predefined nucleotides, and each TSO comprises a UMI unique for the TSO and different from UMIs of other TSOs.

14. The method according to any of the claims 1 to 13, further comprising amplifying the extended cDNA strand using a forward primer and a reverse primer, wherein
the forward primer preferably comprises the amplification primer site and the identification tag, and more preferably comprises, from a 5′ end to a 3′ end, a transposase 5 (Tn5) motif sequence and the identification tag, such as comprises TCGTCGGCAGCGTCAGATGTGTATAAGAGACAGATTGCGCAATG (SEQ ID NO: 6); and
the reverse primer preferably comprises ACGAGCATCAGCAGCATACGA (SEQ ID NO: 5).

15. The method according to claim 14, wherein amplifying the extended cDNA strand is performed simultaneous as the reverse transcription and template switching reaction.

16. The method according to any of the claims 1 to 15, further comprising fragmenting and tagging the extended cDNA strand or an amplified version thereof in a tagmentation process using a transposase and at least one tagging adapter to form tagged cDNA fragments.

17. The method according to claim 16, further comprising amplifying the tagged cDNA fragments in presence of a forward amplification primer and a reverse amplification primer.

18. The method according to claim 17, further comprising sequencing the amplified tagged cDNA fragments by addition of at least one sequencing primer.

19. A method for preparing a cDNA library comprising:
preparing tagged cDNA fragments from RNA molecules, preferably of a single cell, according to any of the claims 16 to 18; and
tuning a percentage of the tagged cDNA fragments corresponding to a 5′ end portion of the extended cDNA strands.

20. The method according to claim 19, wherein tuning the percentage comprises:
controlling an amount of transposase present in the tagmentation process according to any of the claims 16 to 18;
controlling an amount of the extended cDNA strand or there amplified version thereof present in the tagmentation process according to any of the claims 16 to 18; and/or
controlling a reaction time of the tagmentation process according to any of the claims 16 to 18.

21. A kit for preparing complementary deoxyribonucleic acid (cDNA) comprising:
a cDNA synthesis primer configured to hybridize to a ribonucleic acid (RNA) molecule to enable synthesis of a cDNA strand complementary to at least a portion of the RNA molecule to form an RNA-cDNA intermediate; and
a template switching oligonucleotide (TSO) comprising an amplification primer site, an identification tag, a unique molecular identifier (UMI) and multiple predefined nucleotides, wherein the TSO is configured to act as a template in a template switching reaction comprising extension of the cDNA strand to form an extended cDNA strand complementary to the at least a portion of the RNA molecule and the TSO.

22. A method for preparing nucleic acid fragments, the method comprising:
hybridizing a cDNA synthesis primer to a ribonucleic acid (RNA) molecule and synthesizing a cDNA strand complementary to at least a portion of the RNA molecule to form an RNA-cDNA intermediate;
performing a template switching reaction by contacting the RNA-cDNA intermediate with a template switching oligonucleotide (TSO) under conditions suitable for extension of the cDNA strand using the TSO as template to form an extended cDNA strand complementary to the at least a portion of the RNA molecule and the TSO, wherein the TSO comprises an amplification primer site, an identification tag, a unique molecular identifier (UMI) and multiple predefined nucleotides;
producing double-stranded cDNA from the extended cDNA strand; and
fragmenting the double-stranded cDNA to produce nucleic acid fragments comprising a first population of 5′ UMI comprising fragments and a second population of internal fragments.

23. The method according to claim 22, wherein the cDNA synthesis primer comprises a reverse amplification primer site.

24. The method according to any of claims 22 and 23, wherein the cDNA synthesis primer comprises an oligo-dT RNA binding site or a gene specific RNA binding site.

25. The method according to any of claims 22 to 24, wherein producing double-stranded cDNA comprises amplifying.

26. The method according to claim 25, wherein the amplifying comprises employing a forward primer that hybridizes to the TSO amplification primer site and a reverse primer that hybridizes the cDNA synthesis primer comprises a reverse amplification primer site.

27. The method according to any of the preceding claims, wherein the fragmenting comprises tagmenting to produce tagged fragments.

28. The method according to claim 27, wherein the amplification primer site comprises a portion of a transposase motif sequence of the transposase used in the tagmenting.

29. The method according to claim 28, wherein the transposase motif is Tn5.

30. The method according to any of claims 22 to 26, wherein the fragmenting comprises shearing, sonication or enzymatic fragmentation.

31. The method according to claim 30, wherein the method further comprises tagging the first population of 5′ UMI comprising fragments and a second population of internal fragments with tagging adaptors.

32. The method according to claim 31, wherein the tagging adaptors comprises a first tagging adapter comprising a read 1 sequencing primer site and a second tagging adapter comprising a read 2 sequencing primer site.

33. The method according to any of the claims 22 to 32, wherein
hybridizing the cDNA synthesis primer comprises hybridizing, for each RNA molecule of a plurality of RNA molecules, the cDNA synthesis primer to the RNA molecule and synthesizing a respective cDNA strand complementary to at least a portion of the RNA molecule to form a respective RNA-cDNA intermediate; and
performing the template switching reaction comprises performing the template switching reaction by contacting the respective RNA-cDNA intermediate with a respective TSO under conditions suitable for extension of the respective cDNA strand using the respective TSO as template to form a respective extended cDNA strand complementary to the at least a portion of the RNA molecule and the respective TSO, wherein each TSO comprises the amplification primer site, the identification tag, a UMI and the multiple predefined nucleotides, and each TSO comprises a UMI unique for the TSO and different from UMIs of other TSOs.

34. The method according to claim 33, wherein the plurality of RNA molecules is from a single cell.

35. The method according to claim 33, wherein the plurality of RNA molecules is from a plurality of cells.

36. The method according to any of the preceding claims, wherein the method further comprises sequencing the first population of 5′ UMI comprising fragments and a second population of internal fragments.

37. The method according to claim 36, wherein the method further comprises distinguishing sequencing reads of the first population of 5′ UMI comprising fragments from sequencing reads of the internal fragments by the presence of the identification tag sequence.

38. The method according to claim 37, wherein the method further comprises constructing the full-length sequence of the RNA from sequencing reads of both the 5′ UMI comprising and internal fragments.

39. The method according to claim 38, wherein the constructing comprises employing sequencing reads of internal fragments produced from the same RNA from which the 5′UMI comprising fragments were produced.

40. The method according to any of claims 38 and 39, wherein the method further comprises assigning an isoform to the RNA.

41. The method according to any of claims 38 to 40, wherein the method further comprising identifying at least a first SNP of the RNA.

42. The method according to claim 41, wherein the method further comprises identifying at least a second SNP of the RNA.

43. The method according to claim 42, wherein the method further comprises setting a phase relationship of the first and second SNPs.

44. The method according to claims 38 and 39, wherein the method comprises identifying the RNA as the product of a gene fusion.

45. The method according to any of claims 22 to 44, wherein
hybridizing the cDNA synthesis primer comprises hybridizing the cDNA synthesis primer to the RNA molecule and synthesizing the cDNA strand by reverse transcription to form the RNA-cDNA intermediate; and
performing the template switching reaction comprises performing the template switching reaction by contacting the RNA-cDNA intermediate with the TSO under conditions suitable for extension of the cDNA strand by reverse transcription to form the extended cDNA strand.

46. The method according to claim 45, wherein the reverse transcription is conducted in the presence of ribonucleotides, preferably guanine ribonucleotides, at a concentration selected within an interval of from 0.05 mM to 10 mM, preferably within an interval of from 0.1 mM to 3 mM.

47. The method according to any of claims 45 to 46, wherein
the reverse transcription is conducted in the presence of a mixture dATP, dGTP, dTTP and dCTP;
the mixture comprises a same concentration of dATP, dGTP and dTTP and a concentration of dCTP being X mM higher than the same concentration of dATP, dGTP and dTTP; and
X is selected within an interval of from 0.05 mM to 10 mM, preferably within an interval of from 0.1 mM to 3 mM.

48. The method according to any of claims 45 to 47, wherein the reverse transcription is conducted in the presence of a magnesium salt in a concentration selected within an interval of from 0.1 mM to 20 mM, preferably within an interval of from 1 mM to 10 mM, and more preferably within an interval of from 2 mM to 5 mM.

49. The method according to any of the claims 45 to 48, wherein the reverse transcription is conducted in the presence of a chloride salt selected from the group consisting of sodium chloride (NaCl), cesium chloride (CsCl), and a mixture thereof, and is conducted in at least reduced amount of potassium chloride (KCl).

50. The method according to any of the claims 45 to 49, wherein the reverse transcription is conducted in the presence of a polyethylene glycol (PEG) having an average molecular weight selected within an interval of from 300 Da to 100,000 Da, preferably within an interval of from 1,000 to 25,000 Da, and more preferably within an interval of from 7,000 Da to 9,000 Da, such as 8000 Da.

51. A kit for preparing nucleic acid fragments, the kit comprising:
a cDNA synthesis primer configured to hybridize to a ribonucleic acid (RNA) molecule to enable synthesis of a cDNA strand complementary to at least a portion of the RNA molecule to form an RNA-cDNA intermediate and comprising a reverse amplification primer site; and
a template switching oligonucleotide (TSO) comprising an amplification primer site, an identification tag, a unique molecular identifier (UMI) and multiple predefined nucleotides, wherein the TSO is configured to act as a template in a template switching reaction comprising extension of the cDNA strand to form an extended cDNA strand complementary to the at least a portion of the RNA molecule and the TSO.

52. The kit according to claim 51, wherein the cDNA synthesis primer comprises an oligo-dT RNA binding site.

53. The kit according to claim 51, wherein the cDNA synthesis primer comprises a gene specific RNA binding site.

54. The kit according to any of claims 51 to 53, wherein the amplification primer site comprises a portion of a transposase motif sequence.

55. The kit according to claim 54, wherein the transposase motif is Tn5.


SEQUENCE LISTING (84 sequences)

SEQ ID NO: 1 - DNA, 20 bp, Artificial sequence
  synthetic sequence
aatgatacggcgaccaccga

SEQ ID NO: 2 - DNA, 24 bp, Artificial sequence
  synthetic sequence
caagcagaagacggcatacgagat

SEQ ID NO: 3 - DNA, 33 bp, Artificial sequence
  synthetic sequence
acactctttccctacacgacgctcttccgatct

SEQ ID NO: 4 - DNA, 34 bp, Artificial sequence
  synthetic sequence
gtgactggagttcagacgtgtgctcttccgatct

SEQ ID NO: 5 - DNA, 30 bp, Artificial sequence
  synthetic sequence
ccatctcatccctgcgtgtctccgactcag

SEQ ID NO: 6 - DNA, 23 bp, Artificial sequence
  synthetic sequence
cctctctatgggcagtcggtgat

SEQ ID NO: 7 - DNA, 19 bp, Artificial sequence
  synthetic sequence
agatgtgtataagagacag

SEQ ID NO: 8 - DNA, 19 bp, Artificial sequence
  synthetic sequence
ctgtctcttatacacatct

SEQ ID NO: 9 - DNA, 33 bp, Artificial sequence
  synthetic sequence
tcgtcggcagcgtcagatgtgtataagagacag

SEQ ID NO: 10 - DNA, 34 bp, Artificial sequence
  synthetic sequence
gtctcgtgggctcggagatgtgtataagagacag

SEQ ID NO: 11 - DNA, 11 bp, Artificial sequence
  synthetic sequence
attgcgcaatg

SEQ ID NO: 12 - DNA, 30 bp, Artificial sequence
  synthetic sequence
  misc_feature (20)..(27): n is a, c, g, or t
  misc_feature (28)..(30): The nucleotides at positions 28-30 are guanine ribonucleotides, i.e., rGrGrG
agagacagattgcgcaatgnnnnnnnnnnn

SEQ ID NO: 13 - DNA, 21 bp, Artificial sequence
  synthetic sequence
acgagcatcagcagcatacga

SEQ ID NO: 14 - DNA, 24 bp, Artificial sequence
  synthetic sequence
  misc_feature (22)..(22): The nucleotide at position 22 is a thymine that may be repeated p times where p is a positive number selected within an interval of from 10 to 50, preferably from 15 to 45, and more preferably from 20 to 40, such as 30
  misc_feature (23)..(23): The nucleotide at position 23 is selected from the group consisting of A, C and G
  misc_feature (24)..(24): n is a, c, g, or t
acgagcatcagcagcatacgannn

SEQ ID NO: 15 - DNA, 44 bp, Artificial sequence
  synthetic sequence
tcgtcggcagcgtcagatgtgtataagagacagattgcgcaatg

SEQ ID NO: 16 - DNA, 21 bp, Artificial sequence
  synthetic sequence
acgagcatcagcagcatacga

SEQ ID NO: 17 - DNA, 33 bp, Artificial sequence; identical to SEQ ID NO: 9
  synthetic sequence

SEQ ID NO: 18 - DNA, 34 bp, Artificial sequence; identical to SEQ ID NO: 10
  synthetic sequence

SEQ ID NO: 19 - DNA, 20 bp, Artificial sequence
  synthetic sequence
aatgatacggcgaccaccga

SEQ ID NO: 20 - DNA, 42 bp, Artificial sequence
  synthetic sequence
  misc_feature (21)..(28): n is a, c, g, or t
aatgatacggcgaccaccgannnnnnnntcgtcggcagcgtc

SEQ ID NO: 21 - DNA, 24 bp, Artificial sequence
  synthetic sequence
caagcagaagacggcatacgagat

SEQ ID NO: 22 - DNA, 47 bp, Artificial sequence
  synthetic sequence
  misc_feature (25)..(32): n is a, c, g, or t
caagcagaagacggcatacgagatnnnnnnnngtctcgtgggctcgg

SEQ ID NO: 23 - DNA, 30 bp, Artificial sequence; identical to SEQ ID NO: 12
  synthetic sequence
  misc_feature (1)..(1): The nucleotide at position 1 is attached to biotin
  misc_feature (20)..(27): n is a, c, g, or t
  misc_feature (28)..(30): The nucleotides at positions 28 to 30 are guanine ribonucleotides, i.e., rGrGrG

SEQ ID NO: 24 - DNA, 44 bp, Artificial sequence; identical to SEQ ID NO: 15
  synthetic sequence

SEQ ID NO: 25 - DNA, 21 bp, Artificial sequence
  synthetic sequence
acgagcatcagcagcatacga

SEQ ID NO: 26 - DNA, 11 bp, Artificial sequence
  synthetic sequence
attgcgcaatg

SEQ ID NO: 27 - DNA, 53 bp, Artificial sequence
  synthetic sequence
  misc_feature (1)..(1): The nucleotide at position 1 is attached to the 3′ end of Biotin-idSp-idSp-idSp where idSp is Internal 1′,2′-dideoxyribose (dSpacer)
  misc_feature (52)..(52): The nucleotide at position 52 is selected from the group consisting of A, C, and G
  misc_feature (53)..(53): n is a, c, g, or t
acgagcatcagcagcatacgattttttttttttttttttttttttttttttnn

SEQ ID NO: 28 - DNA, 53 bp, Artificial sequence; identical to SEQ ID NO: 27
  synthetic sequence
  misc_feature (1)..(1): The nucleotide at position 1 is attached to biotin
  misc_feature (52)..(52): The nucleotide at position 52 is selected from the group consisting of A, C, and G
  misc_feature (53)..(53): n is a, c, g, or t

SEQ ID NO: 29 - DNA, 53 bp, Artificial sequence; identical to SEQ ID NO: 27
  synthetic sequence
  misc_feature (1)..(1): The nucleotide at position 1 is attached to the 3′ end of biotin-TEG
  misc_feature (52)..(52): The nucleotide at position 52 is selected from the group consisting of A, C, and G
  misc_feature (53)..(53): n is a, c, g, or t

SEQ ID NO: 30 - DNA, 53 bp, Artificial sequence; identical to SEQ ID NO: 27
  synthetic sequence
  misc_feature (52)..(52): The nucleotide at position 52 is selected from the group consisting of A, C and G
  misc_feature (53)..(53): n is a, c, g, or t

SEQ ID NO: 31 - DNA, 52 bp, Artificial sequence
  synthetic sequence
  misc_feature (1)..(1): The nucleotide at position 1 is attached to biotin
  misc_feature (52)..(52): The nucleotide at position 52 is selected from the group consisting of A, C and G
acgagcatcagcagcatacgattttttttttttttttttttttttttttttn

SEQ ID NO: 32 - DNA, 51 bp, Artificial sequence
  synthetic sequence
  misc_feature (1)..(1): The nucleotide at position 1 is attached to biotin
acgagcatcagcagcatacgatttttttttttttttttttttttttttttt

SEQ ID NO: 33 - DNA, 43 bp, Artificial sequence
  synthetic sequence
  misc_feature (1)..(1): The nucleotide at position 1 is attached to biotin
  misc_feature (42)..(42): The nucleotide at position 42 is selected from the group consisting of A, C and G
  misc_feature (43)..(43): n is a, c, g, or t
acgagcatcagcagcatacgattttttttttttttttttttnn

SEQ ID NO: 34 - DNA, 63 bp, Artificial sequence
  synthetic sequence
  misc_feature (1)..(1): The nucleotide at position 1 is attached to biotin
  misc_feature (33)..(33): The nucleotide at position 62 is selected from the group consisting of A, C and G
  misc_feature (62)..(63): n is a, c, g, or t
acgagcatcagcagcatacgattttttttttttttttttttttttttttttttttttttttnn

SEQ ID NO: 35 - DNA, 71 bp, Artificial sequence
  synthetic sequence
  misc_feature (1)..(1): The nucleotide at position 1 is attached to biotin
  misc_feature (70)..(70): The nucleotide at position 70 is selected from the group consisting of A, C and G
  misc_feature (71)..(71): n is a, c, g, or t
acgagcatcagcagcatacgattttttttttttttttttttttttttttttttttttttttttttttttnn

SEQ ID NO: 36 - DNA, 23 bp, Artificial sequence
  synthetic sequence
  misc_feature (1)..(1): The nucleotide at position 1 is attached to the 3′ end of Biotin-idSp-idSp-idSp
  misc_feature (21)..(21): The nucleotide at position 21 is a guanine ribonucleotide
  misc_feature (22)..(22): The nucleotide at position 22 is a guanine ribonucleotide
actggaagagtgccatcaganng

SEQ ID NO: 37 - DNA, 28 bp, Artificial sequence
  synthetic sequence
  misc_feature (1)..(1): The nucleotide at position 1 is attached to biotin
  misc_feature (26)..(26): The nucleotide at position 26 is a guanine ribonucleotide
  misc_feature (27)..(27): The nucleotide at position 27 is an LNA
agagacagattgcgcaatghhhhhhngg

SEQ ID NO: 38 - DNA, 28 bp, Artificial sequence
  synthetic sequence
  misc_feature (1)..(1): The nucleotide at position 1 is attached to the 3′ end of biotin-TEG
  misc_feature (26)..(26): The nucleotide at position 26 is a guanine ribonucleotide
  misc_feature (27)..(27): The nucleotide at position 27 is an LNA
agagacagattgcgcaatghhhhhhngg

SEQ ID NO: 39 - DNA, 28 bp, Artificial sequence
  synthetic sequence
  misc_feature (1)..(1): The nucleotide at position 1 is attached to the 3′ end of biotin-TEG
  misc_feature (26)..(28): The nucleotides from positions 26 to 28 are guanine ribonucleotides
agagacagattgcgcaatghhhhhhnnn

SEQ ID NO: 40 - DNA, 29 bp, Artificial sequence
  synthetic sequence
  misc_feature (1)..(1): The nucleotide at position 1 is attached to the 3′ end of biotin-TEG
  misc_feature (27)..(29): The nucleotides from positions 27 to 29 are guanine ribonucleotides
agagacagattgcgcaatghhhhhhhnnn

SEQ ID NO: 41 - DNA, 29 bp, Artificial sequence
  synthetic sequence
  misc_feature (1)..(1): The nucleotide at position 1 is attached to the 3′ end of biotin-TEG
  misc_feature (27)..(27): The nucleotide at position 27 is a guanine ribonucleotide
  misc_feature (28)..(28): The nucleotide at position 28 is an LNA
agagacagattgcgcaatghhhhhhhngg

SEQ ID NO: 42 - DNA, 22 bp, Artificial sequence
  synthetic sequence
  misc_feature (1)..(1): The nucleotide at position 1 is attached to the 3′ end of biotin-TEG
  misc_feature (20)..(22): The nucleotides at positions 20 to 22 are guanine ribonucleotides
agagacagattgcgcaatgnnn

SEQ ID NO: 43 - DNA, 26 bp, Artificial sequence
  synthetic sequence
  misc_feature (1)..(1): The nucleotide at position 1 is attached to the 3′ end of biotin-TEG
  misc_feature (20)..(23): n is a, c, g, or t
  misc_feature (24)..(26): The nucleotides at positions 24 to 26 are guanine ribonucleotides
agagacagattgcgcaatgnnnnnnn

SEQ ID NO: 44 - DNA, 30 bp, Artificial sequence
  synthetic sequence
  misc_feature (1)..(1): The nucleotide at position 1 is attached to the 3′ end of biotin-TEG
  misc_feature (28)..(30): The nucleotides at positions 27 to 30 are guanine ribonucleotides
agagacagattgcgcaatghhhhhhhhnnn

SEQ ID NO: 45 - DNA, 31 bp, Artificial sequence
  synthetic sequence
  misc_feature (1)..(1): The nucleotide at position 1 is attached to the 3′ end of biotin-TEG
  misc_feature (29)..(31): The nucleotides at positions 29 to 31 are guanine ribonucleotides
agagacagattgcgcaatghhhhhhhhhnnn

SEQ ID NO: 46 - DNA, 32 bp, Artificial sequence
  synthetic sequence
  misc_feature (1)..(1): The nucleotide at position 1 is attached the 3′ end of biotin-TEG
  misc_feature (30)..(32): The nucleotides at position 30 to 32 are guanine ribonucleotides
agagacagattgcgcaatghhhhhhhhhhnnn

SEQ ID NO: 47 - DNA, 30 bp, Artificial sequence
  synthetic sequence
  misc_feature (1)..(1): The nucleotide at position 1 is attached the 3′ end of biotin-TEG
  misc_feature (20)..(23): n is a, c, g, or t
  misc_feature (28)..(30): The nucleotides at positions 28 to 30 are guanine ribonucleotides
agagacagattgcgcaatgnnnnhhhhnnn

SEQ ID NO: 48 - DNA, 28 bp, Artificial sequence
  synthetic sequence
  misc_feature (1)..(1): The nucleotide at position 1 is attached to biotin
  misc_feature (20)..(25): n is a, c, g, or t
  misc_feature (26)..(28): The nucleotides at positions 26 to 28 are guanine ribonucleotides
agagacagattgcgcaatgnnnnnnnnn

SEQ ID NO: 49 - RNA, 28 nt, Artificial sequence
  synthetic sequence
  misc_feature (1)..(1): The nucleotide at position 1 is attached to biotin
  misc_feature (1)..(28): The nucleotides at positions 1 to 28 are rA,rG,rA,rG,rA,rC,rA,rG,rA,rU,rU,rG,rC,rG,rC,rA,rA,rU,rG,rN,rN,rN ,rN,rN,rN,rG,rG,rG, respectively
nnnnnnnnnnnnnnnnnnnnnnnnnnnn

SEQ ID NO: 50 - RNA, 28 nt, Artificial sequence
  synthetic sequence
  misc_feature (1)..(1): The nucleotide at position 1 is attached to biotin
  misc_feature (1)..(28): The nucleotides at positions 1 to 28 are rA, rG, rA, rG, rA, rC, rA, rG, rA, rU, rU, rG, rC, rG, rC, rA, rA, rU, rG, rH, rH, rH, rH, rH, rH, rG, rG, rG, respectively
nnnnnnnnnnnnnnnnnnnnnnnnnnnn

SEQ ID NO: 51 - DNA, 29 bp, Artificial sequence
  synthetic sequence
  misc_feature (1)..(1): The nucleotide at position 1 is attached to biotin
  misc_feature (20)..(26): n is a, c, g, or t
  misc_feature (27)..(29): The nucleotides at positions 27 to 29 are guanine ribonucleotides
agagacagattgcgcaatgnnnnnnnnnn

SEQ ID NO: 52 - DNA, 30 bp, Artificial sequence; identical to SEQ ID NO: 12
  synthetic sequence
  misc_feature (1)..(1): The nucleotide at position 1 is attached to biotin
  misc_feature (20)..(27): n is a, c, g, or t
  misc_feature (28)..(30): The nucleotides at positions 28 to 30 are guanine ribonucleotides

SEQ ID NO: 53 - DNA, 30 bp, Artificial sequence; identical to SEQ ID NO: 12
  synthetic sequence
  misc_feature (20)..(27): n is a, c, g, or t
  misc_feature (28)..(30): The nucleotides at positions 28 to 30 are guanine ribonucleotides

SEQ ID NO: 54 - DNA, 30 bp, Artificial sequence; identical to SEQ ID NO: 12
  synthetic sequence
  misc_feature (1)..(1): The nucleotide at position 1 is attached to biotin
  misc_feature (20)..(27): n is a, c, g, or t
  misc_feature (28)..(28): The nucleotide at position 28 is a ribonucleotide
  misc_feature (29)..(30): The nucleotides at positions 29 to 30 are guanine ribonucleotides

SEQ ID NO: 55 - DNA, 22 bp, Artificial sequence
  synthetic sequence
  misc_feature (1)..(1): The nucleotide at position 1 is attached to biotin
  misc_feature (12)..(19): n is a, c, g, or t
  misc_feature (20)..(22): The nucleotides at positions 20 to 22 are guanine ribonucleotides
attgcgcaatgnnnnnnnnnnn

SEQ ID NO: 56 - DNA, 24 bp, Artificial sequence
  synthetic sequence
  misc_feature (1)..(1): The nucleotide at position 1 is attached to biotin
  misc_feature (14)..(21): n is a, c, g, or t
  misc_feature (22)..(24): The nucleotides at positions 22 to 24 are guanine ribonucleotides
agattgcgcaatgnnnnnnnnnnn

SEQ ID NO: 57 - DNA, 26 bp, Artificial sequence
  synthetic sequence
  misc_feature (1)..(1): The nucleotide at position 1 is attached to biotin
  misc_feature (16)..(23): n is a, c, g, or t
  misc_feature (24)..(26): The nucleotides at positions 24 to 26 are guanine ribonucleotides
acagattgcgcaatgnnnnnnnnnnn

SEQ ID NO: 58 - DNA, 28 bp, Artificial sequence
  synthetic sequence
  misc_feature (1)..(1): The nucleotide at position 1 is attached to biotin
  misc_feature (18)..(25): n is a, c, g, or t
  misc_feature (26)..(28): The nucleotides at positions 26 to 28 are guanine ribonucleotides
agacagattgcgcaatgnnnnnnnnnnn

SEQ ID NO: 59 - DNA, 32 bp, Artificial sequence
  synthetic sequence
  misc_feature (1)..(1): The nucleotide at position 1 is attached to biotin
  misc_feature (22)..(29): n is a, c, g, or t
  misc_feature (30)..(32): The nucleotides at positions 30 to 32 are guanine ribonucleotides
taagagacagattgcgcaatgnnnnnnnnnnn

SEQ ID NO: 60 - DNA, 34 bp, Artificial sequence
  synthetic sequence
  misc_feature (1)..(1): The nucleotide at position 1 is attached to biotin
  misc_feature (24)..(31): n is a, c, g, or t
  misc_feature (32)..(34): The nucleotides at positions 32 to 34 are guanine ribonucleotides
tataagagacagattgcgcaatgnnnnnnnnnnn

SEQ ID NO: 61 - DNA, 36 bp, Artificial sequence
  synthetic sequence
  misc_feature (1)..(1): The nucleotide at position 1 is attached to biotin
  misc_feature (26)..(33): n is a, c, g, or t
  misc_feature (34)..(36): The nucleotides at positions 34 to 36 are guanine ribonucleotides
tgtataagagacagattgcgcaatgnnnnnnnnnnn

SEQ ID NO: 62 - DNA, 32 bp, Artificial sequence
  synthetic sequence
  misc_feature (1)..(1): The nucleotide at position 1 is attached to biotin
  misc_feature (20)..(27): n is a, c, g, or t
  misc_feature (30)..(32): The nucleotides at positions 30 to 32 are guanine ribonucleotides
agagacagattgcgcaatgnnnnnnnnatnnn

SEQ ID NO: 63 - DNA, 28 bp, Artificial sequence
  synthetic sequence
  misc_feature (1)..(1): The nucleotide at position 1 is attached to biotin
  misc_feature (16)..(23): n is a, c, g, or t
  misc_feature (26)..(28): The nucleotides at positions 26 to 28 are guanine ribonucleotides
acagattgcgcaatgnnnnnnnnatnnn

SEQ ID NO: 64 - DNA, 30 bp, Artificial sequence; identical to SEQ ID NO: 12
  synthetic sequence
  misc_feature (1)..(1): The nucleotide at position 1 is attached to biotin
  misc_feature (20)..(27): n is a, c, g, or t
  misc_feature (28)..(30): The nucleotides at positions 28 to 30 are guanine ribonucleotides

SEQ ID NO: 65 - DNA, 28 bp, Artificial sequence
  synthetic sequence
  misc_feature (1)..(1): The nucleotide at position 1 is attached to biotin
  misc_feature (26)..(27): The nucleotides at positions 26 to 27 are guanine ribonucleotides
  misc_feature (28)..(28): The nucleotide at position 28 is an LNA
agagacagattgcgcaatghhhhhhnng

SEQ ID NO: 66 - DNA, 30 bp, Artificial sequence
  synthetic sequence
  misc_feature (1)..(1): The nucleotide at position 1 is attached to biotin
  misc_feature (20)..(27): n is a, c, g, or t
  misc_feature (28)..(29): The nucleotides at positions 28 to 29 are guanine ribonucleotides
  misc_feature (30)..(30): The nucleotide at position 30 is an LNA
agagacagattgcgcaatgnnnnnnnnnng

SEQ ID NO: 67 - DNA, 26 bp, Artificial sequence
  synthetic sequence
  misc_feature (1)..(1): The nucleotide at position 1 is attached to biotin
  misc_feature (20)..(23): The nucleotides at positions 20 to 23 are i5NiTInd (Internal 5-Nitroindole Universal base)
  misc_feature (24)..(26): The nucleotides at positions 24 to 26 are guanine ribonucleotides
agagacagattgcgcaatgnnnnnnn

SEQ ID NO: 68 - DNA, 30 bp, Artificial sequence; identical to SEQ ID NO: 12
  synthetic sequence
  misc_feature (1)..(1): The nucleotide at position 1 is attached to biotin
  misc_feature (20)..(23): The nucleotides at positions 20 to 23 are i5NiTInd (Internal 5-Nitroindole Universal base)
  misc_feature (24)..(27): n is a, c, g, or t
  misc_feature (28)..(30): The nucleotides at positions 28 to 30 are guanine ribonucleotides

SEQ ID NO: 69 - DNA, 30 bp, Artificial sequence; identical to SEQ ID NO: 66
  synthetic sequence
  misc_feature (1)..(1): The nucleotide at position 1 is attached to biotin
  misc_feature (20)..(27): n is a, c, g, or t
  misc_feature (28)..(29): The nucleotides at positions 28 to 29 are guanine ribonucleotides

SEQ ID NO: 70 - DNA, 21 bp, Artificial sequence
  synthetic sequence
acgagcatcagcagcatacga

SEQ ID NO: 71 - DNA, 44 bp, Artificial sequence; identical to SEQ ID NO: 15
  synthetic sequence

SEQ ID NO: 72 - DNA, 29 bp, Artificial sequence
  synthetic sequence
tcgtatgcacgagcatcagcagcatacga

SEQ ID NO: 73 - DNA, 52 bp, Artificial sequence
  synthetic sequence
gcgcaatctcgtcggcagcgtcagatgtgtataagagacagattgcgcaatg

SEQ ID NO: 74 - DNA, 55 bp, Artificial sequence
  synthetic sequence
cattgcgcaattcgtcggcagcgtcagatgtgtataagagacagattgcgcaatg

SEQ ID NO: 75 - DNA, 44 bp, Artificial sequence; identical to SEQ ID NO: 15
  synthetic sequence
  misc_feature (1)..(1): The nucleotide at posiiton 1 is attached to ThioMC6-D (5′ Thiol Modifier C6 S-S)

SEQ ID NO: 76 - DNA, 21 bp, Artificial sequence
  synthetic sequence
  misc_feature (1)..(1): The nucleotide at position 1 is attached to ThioMC6-D (5′ Thiol Modifier C6 S-S)
acgagcatcagcagcatacga

SEQ ID NO: 77 - DNA, 53 bp, Artificial sequence; identical to SEQ ID NO: 27
  synthetic sequence
  misc_feature (1)..(1): The nucleotide at position 1 is attached to biotin
  misc_feature (23)..(23): The nucleotide at position 23 is selected from the group consisting of A, C, and G
  misc_feature (52)..(53): n is a, c, g, or t

SEQ ID NO: 78 - DNA, 30 bp, Artificial sequence; identical to SEQ ID NO: 12
  synthetic sequence
  misc_feature (1)..(1): The nucleotide at position 1 is attached to biotin
  misc_feature (20)..(27): n is a, c, g, or t
  misc_feature (28)..(30): The nucleotides at positions 28 to 30 are guanine ribonucleotides

SEQ ID NO: 79 - DNA, 44 bp, Artificial sequence; identical to SEQ ID NO: 15
  synthetic sequence

SEQ ID NO: 80 - DNA, 21 bp, Artificial sequence
  synthetic sequence
acgagcatcagcagcatacga

SEQ ID NO: 81 - DNA, 11 bp, Artificial sequence
  synthetic sequence
attgcgcaatg

SEQ ID NO: 82 - DNA, 19 bp, Artificial sequence
  synthetic sequence
ctgtctcttatacacatct

SEQ ID NO: 83 - DNA, 16 bp, Artificial sequence
  synthetic sequence
ttccgttcgcgactaa

SEQ ID NO: 84 - DNA, 53 bp, Artificial sequence; identical to SEQ ID NO: 27
  synthetic sequence
  misc_feature (1)..(1): The nucleotide at position 1 is attached to biotin
  misc_feature (52)..(52): The nucleotide at position 52 is seleced from the group consisting of A, C, and G
  misc_feature (53)..(53): n is a, c, g, or t